SEQUENCE LISTING (6 sequences)

SEQ ID NO: 1 - DNA, 20 bp, synthetic construct
  source 1..20: mol_type = other DNA
cgcgatagcgcgaatatatt

SEQ ID NO: 2 - DNA, 19 bp, synthetic construct
  source 1..19: mol_type = other DNA
cacccacgaactcgtcgca

SEQ ID NO: 3 - DNA, 19 bp, synthetic construct
  source 1..19: mol_type = other DNA
gacttcgaccatagctgct

SEQ ID NO: 4 - DNA, 19 bp, synthetic construct
  source 1..19: mol_type = other DNA
catggaagagcctcccgct

SEQ ID NO: 5 - DNA, 19 bp, synthetic construct
  source 1..19: mol_type = other DNA
gacaacaacattcgcagtg

SEQ ID NO: 6 - protein, 8 aa, synthetic construct
DYKDDDDK

(19) United States
(12) Patent Application Publication
(10) Pub. No.: US 2025/0262210 A1
(43) Pub. Date: Aug. 21, 2025

(54) LZTR1 MUTANT TUMORS AND METHODS THEREOF

(71) Applicant: The Trustees of Columbia University in the City of New York, New York, NY (US)

(72) Inventors: Antonio IAVARONE, Sunny Isles Beach, FL (US); Anna LASORELLA, Sunny Isles Beach, FL (US); Aram KO, Miami, FL (US)

(21) Appl. No.: 19/203,455

(22) Filed: May 9, 2025

Related U.S. Application Data
(63) Continuation of application No. PCT/US23/79424, filed on Nov. 10, 2023.
(60) Provisional application No. 63/424,870, filed on Nov. 11, 2022.

(51) Int. Cl. A61K 31/517; A61K 31/502; A61K 31/506; A61P 35/00

(52) U.S. Cl. CPC A61K 31/517; A61K 31/502; A61K 31/506; A61P 35/00

(57) ABSTRACT

The subject matter disclosed herein relates to a method of treating cancer in a subject in need thereof, the method comprising administering to the subject an effective amount of a composition comprising at least one inhibitor of one or more LZTR1 substrates, for example a composition comprising an EGFR inhibitor and an AXL inhibitor.

This application is a continuation of PCT/US2023/079424, filed on Nov. 10, 2023, which claims the benefit of and priority to U.S. Provisional Patent Application No. 63/424,870, filed on Nov. 11, 2022, the content of which are hereby incorporated by reference in their entireties.

### GOVERNMENT SUPPORT

This invention was made with government support under CA178546, CA131126, CA253183, CA239721, CA193313, CA239698, CA179044, CA190891, CA101644, and CA013696 awarded by the National Institutes of Health. The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in XML format and is hereby incorporated by reference in its entirety. Said XML copy, created on May 7, 2025, is named 0019240_01299US2_SL.xml and is 6,125 bytes in size.

### BACKGROUND OF THE INVENTION

To date a full characterization of the function of the LZTR1 protein is still lacking but the interest in LZTR1 function continues to increase due to the growing number of cancer types that are reported to harbor genetic alterations of the LZTR1 gene. For example, LZTR1 mutation and deletions are found in glioblastoma multiforme (GBM), as well as numerous other cancers. Glioblastoma multiforme (GBM) is the most common form of brain cancer and among the most incurable and lethal of all human cancers. There are few available targeted therapies and none that specifically target GBM. The prognosis of GBM remains uniformly poor.

### SUMMARY OF THE INVENTION

In certain aspects, the subject matter described herein provides a method of treating cancer in a subject in need thereof, the method comprising administering to the subject a pharmaceutical composition comprising a therapeutically effective amount of at least one Leucine Zipper-like Transcription Regulator 1 (LZTR1) substrate inhibitor, wherein the cancer is associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, a cell from the cancer or a tumor has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL-3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof.

In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the pharmaceutical composition comprises a first RTK inhibitor and a second RTK inhibitor. In some embodiments, both the first and second RTK inhibitors are small molecule inhibitors. In some embodiments, the pharmaceutical composition comprises an EGFR inhibitor and an AXL inhibitor. In some embodiments, the two RTK inhibitors are osimertinib or a pharmaceutically acceptable salt thereof and bemcentinib or a pharmaceutically acceptable salt thereof. In some embodiments, the two RTK inhibitors are afatinib or a pharmaceutically acceptable salt thereof and bemcentinib or a pharmaceutically acceptable salt thereof. In some embodiments, the pharmaceutical composition comprises a first RTK inhibitor and the method further comprising administering a second pharmaceutical composition comprising a second RTK inhibitor, which is different from the first RTK inhibitor. In some embodiments, the pharmaceutical composition comprises an EGFR inhibitor and the second pharmaceutical composition comprises an AXL inhibitor. In some embodiments, the pharmaceutical composition comprises osimertinib or a pharmaceutically acceptable salt thereof and the second pharmaceutical composition comprises bemcentinib or a pharmaceutically acceptable salt thereof. In some embodiments, the pharmaceutical composition comprises afatinib or a pharmaceutically acceptable salt thereof and the second pharmaceutical composition comprises bemcentinib or a pharmaceutically acceptable salt thereof. In some embodiments, the pharmaceutical composition and second pharmaceutical composition are administered to the subject at the same time. In some embodiments, the pharmaceutical composition and the second pharmaceutical composition are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene. In some embodiments, administration of the first and second RTK inhibitors provides a synergistic effect compared to administration of either inhibitor alone. In some embodiments, the subject is a human.

In certain aspects, the subject matter disclosed herein provides a pharmaceutical composition comprising a therapeutically effective amount of an EGFR inhibitor and an AXL inhibitor.

In some embodiments, the EGFR inhibitor comprises osimertinib or a pharmaceutically acceptable salt thereof and the AXL inhibitor comprises bemcentinib or a pharmaceutically acceptable salt thereof. In some embodiments, the EGFR inhibitor comprises afatinib or a pharmaceutically acceptable salt thereof and the AXL inhibitor comprises bemcentinib or a pharmaceutically acceptable salt thereof.

In some embodiments, the pharmaceutical composition further comprises at least one pharmaceutically acceptable excipient, diluent, and/or carrier. In some embodiments, the pharmaceutical composition is for treating or ameliorating the effects of cancer in a subject wherein the cancer is associated with a loss-of-function mutation in a LZTR1 gene. In some embodiments, administration of the EGFR and AXL inhibitors provides a synergistic effect compared to administration of either inhibitor alone.

In certain aspects, the subject matter described herein provides a kit comprising a first pharmaceutical composition comprising a therapeutically effective amount of an EGFR inhibitor and a second pharmaceutical composition comprising a therapeutically effective amount of an AXL inhibitor.

In some embodiments, the EGFR inhibitor comprises osimertinib or a pharmaceutically acceptable salt thereof and the AXL inhibitor comprises bemcentinib or a pharmaceutically acceptable salt thereof. In some embodiments, the EGFR inhibitor comprises afatinib or a pharmaceutically acceptable salt thereof and the AXL inhibitor comprises bemcentinib or a pharmaceutically acceptable salt thereof. In some embodiments, each pharmaceutical composition further comprises at least one pharmaceutically acceptable excipient, diluent, and/or carrier. In some embodiments, the combination therapy is for treating or ameliorating the effects of cancer in a subject wherein the cancer is associated with a loss-of-function mutation in a LZTR1 gene. In some embodiments, administration of the combination therapy provides a synergistic effect compared to administration of either pharmaceutical composition alone. In some embodiments, the first and second pharmaceutical compositions are packaged together with instructions for their use.

In certain aspects, the subject matter described herein provides a method of decreasing growth of a solid tumor in a subject in need thereof, the method comprising administering to the subject a pharmaceutical composition comprising a therapeutically effective amount of at least one Leucine Zipper-like Transcription Regulator 1 (LZTR1) substrate inhibitor, wherein the composition decreases the size of the solid tumor, and wherein the tumor is associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, a cell from the cancer or a tumor has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof. In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the pharmaceutical composition comprises a first RTK inhibitor and a second RTK inhibitor. In some embodiments, both the first and second RTK inhibitors are small molecule inhibitors. In some embodiments, the pharmaceutical composition comprises an EGFR inhibitor and an AXL inhibitor. In some embodiments, the two RTK inhibitors are osimertinib or a pharmaceutically acceptable salt thereof and bemcentinib or a pharmaceutically acceptable salt thereof. In some embodiments, the two RTK inhibitors are afatinib or a pharmaceutically acceptable salt thereof and bemcentinib or a pharmaceutically acceptable salt thereof. In some embodiments, the pharmaceutical composition comprises a first RTK inhibitor and the method further comprises administering a second pharmaceutical composition comprising a second RTK inhibitor, which is different from the first RTK inhibitor. In some embodiments, the pharmaceutical composition comprises an EGFR inhibitor and the second pharmaceutical composition comprises an AXL inhibitor. In some embodiments, the pharmaceutical composition comprises osimertinib or a pharmaceutically acceptable salt thereof and the second pharmaceutical composition comprises bemcentinib or a pharmaceutically acceptable salt thereof. In some embodiments, the pharmaceutical composition comprises afatinib or a pharmaceutically acceptable salt thereof and the second pharmaceutical composition comprises bemcentinib or a pharmaceutically acceptable salt thereof. In some embodiments, the pharmaceutical composition and the second pharmaceutical composition are administered to the subject at the same time. In some embodiments, the pharmaceutical composition and the second pharmaceutical composition are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene.

In some embodiments, administration of the first and second RTK inhibitors provides a synergistic effect compared to administration of either inhibitor alone. In some embodiments, the subject is a human.

In certain aspects, the subject matter described herein provides a method of effecting cancer cell death, the method comprising contacting the cancer cell with an effective amount of at least one Leucine Zipper-like Transcription Regulator 1 (LZTR1) substrate inhibitor, wherein the cancer cell is from a cancer associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, the cancer cell has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof. In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the at least one LZTR1 substrate inhibitor comprises a first RTK inhibitor and a second RTK inhibitor. In some embodiments, both the first and second RTK inhibitors are small molecule inhibitors. In some embodiments, the first and second RTK inhibitors comprise an EGFR inhibitor and an AXL inhibitor, respectively. In some embodiments, the first and the second RTK inhibitors are osimertinib or a pharmaceutically acceptable salt thereof and bemcentinib or a pharmaceutically acceptable salt thereof. In some embodiments, the first and the second RTK inhibitors are afatinib or a pharmaceutically acceptable salt thereof and bemcentinib or a pharmaceutically acceptable salt thereof.

In some embodiments, the at least one LZRT1 substrate inhibitor comprises a first RTK inhibitor and the method further comprises administering a second pharmaceutical composition comprising a second RTK inhibitor, which is different from the first RTK inhibitor. In some embodiments, the least one LZRT1 substrate inhibitor an EGFR inhibitor and the second pharmaceutical composition comprises an AXL inhibitor. In some embodiments, the least one LZRT1 substrate inhibitor comprises osimertinib or a pharmaceutically acceptable salt thereof and the second pharmaceutical composition comprises bemcentinib or a pharmaceutically acceptable salt thereof. In some embodiments, the least one LZRT1 substrate inhibitor comprises afatinib or a pharmaceutically acceptable salt thereof and the second pharmaceutical composition comprises bemcentinib or a pharmaceutically acceptable salt thereof. In some embodiments, the first and second LZRT1 substrate inhibitors are administered to the subject at the same time. In some embodiments, the first and second LZRT1 substrate inhibitor are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene.

In certain aspects, the subject matter described herein provides a method for detecting the presence of a LZTR1 protein in a subject, the method comprising: (a) obtaining a biological sample from the subject; and (b) detecting whether or not there is a LZTR1 protein present in the subject.

In some embodiments, the detecting comprises measuring LZRT1 protein levels by an antibody directed to the LZRT1 protein, Western blot using an antibody directed to the LZRT1 protein, ELISA using an antibody directed to the LZRT1 protein, mass spectroscopy, or a combination thereof. In some embodiments, the method further comprises administering to the subject a pharmaceutical composition comprising a therapeutically effective amount of at least one RTK inhibitor if the LZTR1 protein is not present in the sample.

In certain aspects, the subject matter described herein provides a method for detecting the presence of a mutant LZTR1 protein in a subject, the method comprising: (a) obtaining a biological sample from the human subject; and (b) detecting whether or not there is a nucleic acid sequence encoding a mutant LZTR1 protein in the subject.

In some embodiments, the detecting comprises using hybridization, amplification, or sequencing techniques to detect the mutant LZTR1 protein. In some embodiments, the LZTR1 mutation comprises one or more nucleotide insertion, deletion, or substitution mutations in the LZTR1 gene or any combination thereof. In some embodiments, the LZTR1 mutation comprises a deletion of at least a portion of the LZTR1 gene. In some embodiments, the LZTR1 mutation is located in a BTB-Back domain of the LZTR1 gene. In some embodiments, the LZTR1 mutation is V456G, P520L, L591R, R688C, R810W, or S813I. In some embodiments, the LZTR1 mutation is located in a Kelch domain of the LZTR1 gene. In some embodiments, the mutation is H71R, P115L, S122L, R170Q, L187R, M202R, or M400R. In some embodiments, the LZTR1 mutation is located in a CUL-3-interacting domain of the LZTR1 gene. In some embodiments, the LZTR1 mutation is located in a substrate-interacting domain of the LZTR1 gene. In some embodiments, the method further comprises administering to the subject a pharmaceutical composition comprising a therapeutically effective amount of at least one RTK inhibitor if a nucleic acid sequence encoding a mutant LZTR1 protein is present in the sample.

In certain aspects, the subject matter described herein provides a diagnostic kit for determining whether a sample from a subject exhibits a presence of a nucleic acid encoding a mutant LZTR1 protein, the kit comprising at least one oligonucleotide that specifically hybridizes to the nucleic acid encoding the mutant LZTR1.

In some embodiments, the at least one oligonucleotide comprises a set of nucleic acid primers or in situ hybridization probes. In some embodiments, the primers prime a polymerase reaction only when a mutant LZTR1 protein is present.

In some embodiments, the LZTR1 mutation comprises one or more nucleotide insertion, deletion, or substitution mutations in the LZTR1 gene or any combination thereof. In some embodiments, the LZTR1 mutation comprises a deletion of at least a portion of the LZTR1 gene. In some embodiments, the LZTR1 mutation is located in a BTB-Back domain of the LZTR1 gene. In some embodiments, the LZTR1 mutation is V456G, P520L, L591R, R688C, R810W, or S813I. In some embodiments, the LZTR1 mutation is located in a Kelch domain of the LZTR1 gene. In some embodiments, the mutation is H71R, P115L, S122L, R170Q, L187R, M202R, or M400R. In some embodiments, the LZTR1 mutation is located in a CUL-3-interacting domain of the LZTR1 gene. In some embodiments, the LZTR1 mutation is located in a substrate-interacting domain of the LZTR1 gene. In some embodiments, the determining comprises gene sequencing, selective hybridization, selective amplification, gene expression analysis, or a combination thereof.

### BRIEF DESCRIPTION OF THE FIGURES

FIGS. 1A-F. Integration of the proteomic approaches identifies candidate substrates of LZTR1 for ubiquitylation and degradation. A, Schematic representation of the proteomic methods utilized to identify substrates of LZTR1. For quantitative ubiquitin diGly proteomics, Lztr1fl/fl-Rosa-CreER astrocytes expressing doxycycline-inducible LZTR1 were treated with 4-OHT to induce Lztr1 deletion and treated with doxycycline or vehicle in the presence or in the absence of the proteasome inhibitor MG132 for 8 hrs. Cell lysates were immunoprecipitated with anti-diGly lysine antibody and diGly peptides analyzed by LC/MS/MS (panel 1). For Tandem Mass Tag (TMT) protein quantification, two Lztr1+/+ and two Lztr1−/− MEFs were used (panel 2). For immunoprecipitation/mass spectrometry analysis, the cell lysates of U87 expressing LZTR1 wild type-FLAG, LZTR1 R810W-FLAG or the empty vector (EV) were immunoprecipitated with the FLAG-M2 affinity matrix and eluted with FLAG peptide. Co-immunoprecipitated proteins were analyzed by LC/MS/MS (panel 3). B, Scatter plot of the hits from the ubiquitin assay by their localization probability (x-axis) and the intensity fold change between LZTR1 expression and control (y-axis). 1076 ubiquitinated candidates were selected by localization probability >0.9 and fold change >2.5. C, Heatmap showing the normalized protein abundance (z-score) resulting from TMT quantification of proteins enriched in Lztr1−/− compared to Lztr1+/+ cells (fold change >1.5, n=33). Proteins are ranked by fold change. D, Heatmap showing 39 top scoring LZTR1 interacting proteins from immunoprecipitation/mass spectrometry ranked by LZTR1-RW PMS. Candidate LZTR1 interactors were selected by their Peptide Spectrum Matches (PSM) in the different U87 transfectants: PSM=0 in empty vector (EV), PSM>0 in cells expressing LZTR1 wild type (WT) and LZTR1 R810W mutant (RW), and PSM fold change >2 between LZTR1 RW and LZTR1 wild type expressing cells (n=184). E, Venn diagrams highlighting the overlapping of candidate LZTR1 protein substrates selected by the analysis of quantitative ubiquitin diGly proteomics (1076), TMT cellular protein quantification (33), immunoprecipitation/mass spectrometry (184). F, Box plot showing the TMT reporter ion intensity of EGFR and AXL peptides in Hel a transduced with non-targeting sgRNA or sgRNA targeting LZTR1. Box plots span the first to the third quartile. Comparisons between groups were analyzed by the Mann-Whitney Wilcoxon test.

FIGS. 2A-S. LZTR1 interacts with and destabilizes EGFR and AXL. A, Lztr1fl/fl-Rosa-CreER astrocytes were treated with ethanol or 4-OHT. Cell lysates were analyzed by western blot. B, Western blot analysis of cell lysates from Lztr1 knockout SW10 cells, established by CRISPR/Cas9 system. C, CRISPR/Cas9 mediated LZTR1 knockout U87 cells analyzed by western blot. D, CRISPR/Cas9 mediated LZTR1 knockout Hela cells analyzed by western blot. Two different clones for two different sgLZTR1 were analyzed. E, Lztr1 knockout SW10 cells were reconstituted with LZTR1. Cell lysates were analyzed by western blot. F, HEK 293T cells co-expressing EGFR-GFP with increasing amounts of LZTR1-FLAG were analyzed by western blot. G, HEK 293T cells co-expressing AXL-HA with increasing amounts of LZTR1-FLAG were analyzed by western blot. H, LZTR1 knockout Hela cells were treated with cycloheximide (CHX) for the indicated times. Cell lysates were analyzed by western blot. I, Quantification of EGFR for the experiment in H. J, LZTR1 knockout Hela cells were treated with CHX for the indicated times. Cell lysates were analyzed by western blot. K, Quantification of AXL for the experiment in J. L, HEK 293T cells co-expressing EGFR-GFP and LZTR1-FLAG were treated with CHX for the indicated times. Cell lysates were analyzed by western blot. M, Quantification of EGFR-GFP for the experiment in L. N, HEK 293T cells co-expressing AXL-HA and LZTR1-FLAG were treated with CHX for the indicated times. Cell lysates were analyzed by western blot. O, Quantification of AXL-HA for the experiment in N. P, HEK 293T cells were transfected with GFP, EGFR-GFP and LZTR1-FLAG as indicated. Cell lysates were immunoprecipitated with GFP antibody and analyzed by western blot. Q, HEK 293T cells were transfected with FLAG, EGFR-GFP and LZTR1-FLAG as indicated. Cell lysates were immunoprecipitated with FLAG affinity matrix and analyzed by western blot. R, HEK 293T cells were transfected with HA, AXL-HA and LZTR1-FLAG. Cell lysates were immunoprecipitated with HA affinity matrix and analyzed by western blot. S, HEK 293T cells were transfected with FLAG, AXL-HA and LZTR1-FLAG. Cell lysates were immunoprecipitated with FLAG affinity matrix and analyzed by western blot. NT, non-targeting; WCL, whole cell lysates.

FIGS. 3A-L. LZTR1 mediates ubiquitylation and lysosome-mediated degradation of EGFR and AXL. A, HEK 293T cells were transfected with EGFR-GFP and LZTR1-FLAG and treated with bafilomycin A1 or bortezomib. Lysates were analyzed by western blot. B, HEK 293T cells were transfected with AXL-HA and LZTR1-FLAG and treated with bafilomycin A1 or bortezomib. Lysates were analyzed by western blot. C, HEK 293T cells were transfected with EGFR-GFP and LZTR1-FLAG and treated with MLN4924. Cell lysates were analyzed by western blot. D, HEK 293T cells were transfected with AXL-HA and LZTR1-FLAG and treated with MLN4924. Cell lysates were analyzed by western blot. E, HEK 293T cells were transfected with EGFR-GFP, and HA-ubiquitin in the absence or the presence of LZTR1-FLAG and treated with MLN4924 or vehicle as indicated. Cell lysates were immunoprecipitated with HA affinity gel in denaturing condition and analyzed by western blot with the indicated antibodies. F, Hela cells were transfected as indicated and treated with Chloroquine (CQ) or MG132. Cell lysates were immunoprecipitated with normal IgG or AXL antibody in denaturing condition and immunoblotted with the indicated antibodies. G, Hela cells were transfected as indicated and treated with Chloroquine (CQ). Cell lysates were immunoprecipitated with AXL antibody in denaturing condition and immunoblotted with the indicated antibodies. H, HEK 293T cells were transfected with EGFR-GFP, LZTR1-FLAG and HA-ubiquitin as indicated and treated with Bafilomycin A1. Cell lysates were immunoprecipitated with EGFR antibodies in denaturing condition and analyzed by western blot with the indicated antibodies. I, HEK 293T cells co-expressing EGFR-GFP with FLAG-tagged Kelch domain mutants of LZTR1 were analyzed by western blot. J, HEK 293T cells co-expressing AXL-HA with FLAG-tagged Kelch domain mutants of LZTR1 were analyzed by western blot. K, HEK 293T cells co-expressing EGFR-GFP with FLAG-tagged BTB-BACK domain mutants of LZTR1 were analyzed by western blot. L, HEK 293T cells co-expressing AXL-HA with FLAG-tagged BTB-BACK domain mutants of LZTR1 were analyzed by western blot. Experiments were repeated three times with similar results. WCL, whole cell lysates; BFLMA1, Bafilomycin A1; BRTZ, Bortezomib.

FIGS. 4A-J. LZTR1 stimulates EGF-mediated EGFR degradation. A, HEK 293T cells were transfected with EGFR-GFP and LZTR1-FLAG. Cells were serum starved and treated with Chloroquine for 2 hours before the addition of EGF or vehicle. Cell lysates were immunoprecipitated with GFP antibody and analyzed by western blot. The asterisk indicates non-specific bands. B, HEK 293T cells were transfected with AXL-HA and LZTR1-FLAG. Cells were serum starved and treated with Chloroquine for 2 hours before the addition of Gas6 or vehicle. Cell lysates were immunoprecipitated with HA antibody and analyzed by western blot. C, HEK 293T cells were transfected with EGFR WT-GFP or EGFR-K721R-GFP mutant and LZTR1-FLAG. Cells were serum starved, treated with Chloroquine for 2 hours before addition of EGF or vehicle for 15 min. Cell lysates were immunoprecipitated with GFP antibody and analyzed by western blot. D, HEK 293T cells were transfected with EGFR-GFP and LZTR1-FLAG or the empty vector. Cells were serum starved and treated with EGF for the indicated times. Cell lysates were analyzed by western blot. E, HEK 293T cells were transfected with AXL-HA and LZTR1-FLAG or the empty vector. Cells were serum starved and treated with Gas6 for the indicated times. Cell lysates were analyzed by western blot. F, Lztr1+/+ and Lztr1−/− MEFs were serum starved and treated with EGF for the indicated times. Cell lysates were analyzed by western blot. G, Quantification of EGFR-GFP for the experiment in F. H, Hela cells transduced with non-targeting sgRNA (NT) or sgRNA targeting LZTR1 were stimulated with increasing concentration of EGF and analyzed by western blot. I, Hela cells transduced as in H were stimulated with increasing concentration of epiregulin and analyzed by western blot. J, Hela cells transduced as in H were stimulated with 800 ng/ml of Gas6 for the indicated time and analyzed by western blot. Experiments were repeated at least twice with similar results. WCL, whole cell lysates; NT, non-targeting.

FIGS. 5A-H. Loss of LZTR1 impairs EGFR and AXL trafficking to the lysosome. A, CRISPR/Cas9 mediated LZTR1 knockout and control Hela cells transduced with pLOC-EGFR were serum starved and treated with Alexa-Fluor-647 conjugated EGF. Cells were fixed 5 and 45 min after treatment and immunostained using the EEA1 antibody. B, Quantification by the Pearson's correlation coefficient of co-localized Alexa-Fluor-647-EGF and EEA1 at the indicated times after EGF stimulation. C, Cells treated as in A were immunostained using Lamp1 antibody. D, Quantification by the Pearson's correlation coefficient of co-localized of Alexa-Fluor-647-EGF and Lamp1 at the indicated times after EGF stimulation. E, CRISPR/Cas9 mediated LZTR1 knockout and control Hela cells were serum starved and treated with Gas6. Cells were fixed 15 and 60 min after treatment and immunostained using the EEA1 and AXL antibodies. F, Quantification by the Pearson's correlation coefficient of co-localized AXL and EEA1 at the indicated times after Gas6 stimulation. G, Cells treated as in E were immunostained using Lamp1 and AXL antibodies. H, Quantification by the Pearson's correlation coefficient of co-localized AXL and Lamp1 at the indicated times after Gas6 stimulation. Box plots in B, D, F and H span the first to the third quartile and whiskers indicate the smallest and largest values; n>40 cells per sample. Comparisons between groups were analyzed by t-test (two-tailed, unequal variance). Experiments were repeated three times with similar results. sgNT data is shown in the left-most bars for each condition and sgLZTR1 data is shown in the right-most bars for each condition.

FIGS. 6A-H. LZTR1 functions as a tumor suppressor in the mouse peripheral nervous system. A, Kaplan Meier analysis of the survival of Lztr1fl/fl;Cdkn2A fl/fl;GFAP-Cre mice and controls. B, Incidence of tumors/tumor phenotypes in Lztr1fl/fl;Cdkn2A fl/fl;GFAP-Cre mice. C, Representative microphotographs of H & E and immunophenotypic characterization of tumors in Lztr1fl/fl;Cdkn2A fl/fl;GFAP-Cre mice exhibiting schwannoma-like features (S100B, SOX10, Calretinin positivity) and EGFR and AXL protein expression. D, Microphotographs of immunophenotypic characterization of cells from #E0954 schwannoma-like tumor (Lztr1fl/fl;Cdkn2A fl/fl;GFAP-Cre) using S100β, Calretinin, and EGFR and AXL antibodies. E, Mouse schwannoma-like tumor cells were reconstituted with LZTR1. Cell lysates were analyzed by western blot. F, Proliferation curves of the same cells in E. Data are mean±s.d. of n≥3 replicates. **, p<0.001; ***, p<0.0001. G, Representative microphotographs of immunohistochemistry for EGFR and AXL of human sporadic (non-syndromic) schwannomas (left panel), schwannomas from patients carrying germline alteration of LZTR1 (middle panel), or patients carrying germline mutation of SMARCB1 (right panel). H, Quantitative analysis of EGFR and AXL protein expression for the experiment in G (t-test, two-tailed, unequal variance). The left-most data are for “sporadic,” the middle data set are for “LZTR1 germline mutant,” and the right-most data are for “SMARCB1 germline mutant.”

FIGS. 7A-G. LZTR1 inactive tumors exhibit vulnerability to concurrent EGFR and AXL inhibition. A, Evaluation of drug combination effect using the Bliss score for treatment with Afatinib and Bemcentinib of schwannoma-like tumor cells from Lztr1fl/fl;Cdkn2A fl/fl;GFAP-Cre mouse. B, Evaluation of drug combination effect using the Bliss score for treatment with Osimertinib and Bemcentinib of schwannoma-like tumor cells from Lztr1fl/fl;Cdkn2A fl/fl;GFAP-Cre mouse. C, Evaluation of drug combination effect using the Bliss score for treatment with Osimertinib and Bemcentinib of FGFR3-TACC3 expressing mouse cancer cells (Lztr1 wild type cells). D, Cells from schwannoma-like tumor of Lztr1fl/fl;Cdkn2A fl/fl;GFAP-Cre mice were treated with Osimertinib and Bemcentinib as indicated and analyzed by western blot. E, Survival curve of mice injected subcutaneously with mouse schwannoma-like tumor cells. Mice were treated with bemcentinib at 50 mg/kg body weight, afatinib at 10 mg/kg body weight, bemcentinib at 50 mg/kg body weight plus afatinib at 10 mg/kg body weight (5 days in/one day off regimen), or DMSO as control (at least 7 mice for each treatment group); p-values are from the log-rank test. F, Mice were treated with bemcentinib at 50 mg/kg body weight, osimertinib at 5 mg/kg body weight, bemcentinib at 50 mg/kg body weight plus osimertinib at 5 mg/kg body weight (5 days in/one day off regimen), or DMSO as control (10 mice for each treatment group). Tumor size was measured every 2 days. Shown is the scatter plot of tumor size in each mouse under study evaluated after the administration of 12 drug doses; p values are from the non-parametric t-test. The left-most data are for “control,” the second to left data set are for “Bemcentinib,” and second to right-most data are for “Osimertinib,” and the left-most data are for “Combination.” G, Survival curve of the same mouse cohorts presented in F.

FIGS. 8A-E. LZTR1 regulates EGFR and AXL protein stability. A, Western blot analysis and semiquantitative RT-PCR for EGFR of Hela cells transduced with non-targeting sgRNA (NT) or two independent sgRNA targeting LZTR1. B, Western blot analysis and semiquantitative RT-PCR for AXL of Hela cells transduced with non-targeting sgRNA (NT) or two independent sgRNA targeting LZTR1. The asterisk indicates non-specific bands. C, Lztr1^{fl/fl}-Rosa-CreER astrocytes from two independent mice (#1 and #2) were treated with ethanol or 4-OHT. Cell lysates were analyzed by western blot. D, Western blot analysis of Hela cells transduced with non-targeting sgRNA (NT) or two independent sgRNA targeting LZTR1. E, U87 cells were infected with increasing amount of lentivirus containing LZTR1-FLAG and selected with blasticidine. Cell lysates were analyzed by western blot.

FIGS. 9A-D. The Kelch domain of LZTR1 and carboxyl tail of EGFR are required for their interaction. A, Schematic representation of deletion constructs of EGFR. B, GST pull-down assay using GST-EGFR deletion mutants and lysates prepared from HEK 293T cell expressing LZTR1-FLAG. Reactions were analyzed by western blot using FLAG antibody. Membranes were analyzed by western blot using GST antibody as a control for GST fusion proteins included in each reaction. C, Schematic representation of deletion constructs of LZTR1. D, GST pull-down assay using GST-LZTR1 full length or deletion mutants and lysates prepared from HEK 293T cells expressing EGFR-GFP. Reactions were analyzed by western blot using EGFR antibody. Membranes were analyzed by western blot using GST antibody as control for GST fusion proteins included in each reaction. Experiments were repeated three times with similar results.

FIGS. 10A-G. Mechanism of LZTR1-mediated degradation of RIT1 and the effect of LZTR1 mutations. A, HEK 293T cells were transfected with FLAG-RIT1 and LZTR1-FLAG and treated with bafilomycin A1 or bortezomib. Lysates were analyzed by western blot. B, 293T cells were transfected with FLAG-RIT1, EGFR-MYC, AXL-HA and LZTR1-GFP as indicated. Cell lysates were immunoprecipitated with FLAG affinity matrix and analyzed by western blot. C, HEK 293T cells were transfected with FLAG-RIT1 and LZTR1-FLAG and treated with MLN4924. Cell lysates were analyzed by western blot. D, Schematic representation of Kelch domain or BTB-BACK domain mutations of LZTR1E, HEK 293T cells were transfected with EGFR-GFP and FLAG-tagged Kelch domain mutants of LZTR1. Cell lysates were immunoprecipitated with FLAG affinity matrix and analyzed by western blot. F, HEK 293T cells were transfected with FLAG-RIT1 and FLAG-tagged Kelch domain mutants of LZTR1. Cell lysates were analyzed by western blot. G, HEK 293T cells were transfected with EGFR-GFP and LZTR1-FLAG or the empty vector. Cells were serum starved and treated with Chloroquine for 2 hours before the addition of EGF for the indicated times. Cell lysates were immunoprecipitated with EGFR antibody in denaturing condition and analyzed by western blot.

FIGS. 11A-B. LZTR1 co-localized with EGFR and AXL at the cellular membrane upon ligand stimulation. A, Hela cells co-expressing EGFR-GFP and LZTR1-FLAG were exposed to EGF for 5 min. at 40 C, fixed with paraformaldehyde, immunostained with FLAG antibody and analyzed by confocal microscopy for co-localization of EGFR-GFP and LZTR1-FLAG. Nuclei were counterstained with DAPI. Two different cells are presented in the upper and lower left panels with each dashed square highlighting the area that is presented at higher magnification in the right panel. Experiments were repeated two times with similar results. B, Hela cells expressing LZTR1-FLAG were exposed to GAS6 for 5 min. at 40 C, fixed with paraformaldehyde, immunostained with AXL and FLAG antibodies and analyzed by confocal microscopy for co-localization of AXL and LZTR1-FLAG. Nuclei were counterstained with DAPI. Two different cells are presented in the upper and lower left panels with each dashed square highlighting the area that is presented at higher magnification in the right panel. Experiments were repeated two times with similar results.

FIGS. 12A-D. Phenotype alterations in the liver and brain of Lztr1-deleted mice. A, Representative microphotographs of H & E staining and Caspase-3 immunostaining of E13.5 liver from Lztr1+/+ and Lztr1−/− embryos show massive apoptotic cell death in the Lztr1-deleted liver. B, Representative microphotographs of H & E staining of E13.5 telencephalon from Lztr1+/+ and Lztr1−/− embryos. C, Quantification of ventricular/subventricular zone (VZ/SVZ), intermediate zone (IZ) and cortical plate (CP) of E13.5 Lztr1+/+ (n=3) and Lztr1−/− embryos (n=5). Comparisons between groups were analyzed by t-test with Welch correction (two-tailed, unequal variance). The data at the bottom of each bar are for “VZ/SVZ,” the data in the middle of each bar are for “IZ,” and the data at the top of each bar are for “CP.” D, Representative microphotographs of EGFR and AXL immunofluorescence of E14.5 dorsal root ganglia (DRG) from Lztr1+/+ and Lztr1−/− embryos.

FIGS. 13A-E. EGFR and AXL protein expression in the brain and immunohistochemical characterization of tumors of Lztr1fl/fl;Cdkn2afl/fl;GFAP-Cre+ mice. A, Representative microphotographs of EGFR staining of E13.5 brain from Lztr1+/+ and Lztr1−/− embryos. B, Representative microphotographs of AXL staining of E13.5 brain from Lztr1+/+ and Lztr1−/− embryos. The left-most data are for “LZTR1 germline mutant” and the right-most data are for “Sporadic.” C, Semiquantitative characterization of the PNS tumors by immunohistochemistry. D, Microphotograph of H & E (left panel) and EGFR immunofluorescence (right panel) of one representative MPNST. E, Microphotograph of H&E (left panel) and MPO immunofluorescence (right panel) of the spleen infiltrated by myeloid malignant cells. Arrowheads indicate dysplastic megakaryocytes.

FIGS. 14A-F. Protein expression of LZTR1 substrates and pathway analysis in human schwannoma A, Representative microphotographs of immunostaining for RIT1, KRAS and HRAS of human schwannomas from patients carrying germline alteration of LZTR1 (left panel) or patient lacking LZTR1 germline lesions (right panel). B, Quantitative analysis of RIT1, KRAS and HRAS protein expression for the experiment in A (t-test, two-tailed, unequal variance). C, Gene expression heatmap including the top and bottom 100 genes differentially expressed between LZTR1 germline mutant (n=10) and wild type (n=14) schwannoma from Mansouri et al. D, EGFR and RTK specific programs are activated in the LZTR1 germline mutant schwannoma subset (Mann-Whitney-Wilcoxon Gene Set Test, GST-MWW NES>0.58 and FDR<0.05; significant terms are indicated by the asterisk). E, Heatmap of single-sample enrichment score for EGFR and RTK gene signatures (significant terms are indicated by the asterisk, MWW p-value <0.05). F. Boxplot of single-sample enrichment score for significant EGFR and RTK gene signatures in LZTR1 germline mutant (left-hand bars) and wild type (right-hand bar) schwannoma (MWW p-value <0.05). EGF, Epidermal growth factor; RPTK, Receptor protein tyrosine kinase; PTK, Protein tyrosine kinase.

FIGS. 15A-I. Vulnerability of LZTR1 inactive cells to combination treatment of EGFR and AXL inhibitors. A, Schwannoma-like tumor cells from Lztr1fl/fl;Cdkn2A fl/fl;GFAPCre mice were treated with osimertinib and afatinib as indicated. Cell lysates were analyzed by western blot. B, Quantitative analysis of phospho-EGFR for the experiment in A. C, Microphotographs of colony forming assay of schwannoma-like tumor cells from Lztr1fl/fl;Cdkn2A fl/fl;GFAP-Cre mice after treatment with osimertinib, bemcentinib or the combination. Cells were stained with crystal violet. D, Quantification of colonies in the experiment in C. Scatter plot indicates median and whiskers indicate the smallest and largest values (n=3, ttest, two-tailed, unequal variance). E, Microphotographs of colony forming assay schwannoma like tumor cells from Lztr1fl/fl;Cdkn2A fl/fl;GFAP-Cre mice after treatment with afatinib, bemcentinib or the combination. Cells were stained with crystal violet. F, Quantification of colonies in the experiment in E. Scatter plot indicates median and whiskers indicate the smallest and largest values (n=3, t-test, two-tailed, unequal variance). G, Microphotographs of colony forming assay of astrocytes treated with 4-OHT or Ethanol (left panel). Quantification of the experiments in the right panel; Scatter plot indicates median and whiskers indicate the smallest and largest values (n=3, t-test, two-tailed, unequal variance). H, Evaluation of drug combination effect using the Bliss score for treatment with osimertinib and bemcentinib of mouse Lztr1fl/fl;Rosa-Cre-ER astrocytes treated with 4-OHT (Lztr1-deleted cells, left panel) or ethanol (wild type cells, right panel). I, Evaluation of drug combination effect using the Bliss score for treatment with afatinib and bemcentinib of mouse Lztr1fl/fl;Rosa-Cre-ER astrocytes treated with 4-OHT (Lztr1-deleted cells, left panel) or ethanol (wild type cells, right panel).

### DETAILED DESCRIPTION OF THE INVENTION

In some embodiments, the subject matter described herein relates to a combination therapy including EGFR inhibitors and AXL inhibitors in the treatment of cancer. In some embodiments, the cancer is glioblastoma. In some embodiments, the cancer is hepatocellular cancer. In some embodiments, the cancer is esophagogastric cancer. In some embodiments, the cancer is colorectal carcinoma. In some embodiments, the cancer is breast carcinoma. In some embodiments, the cancer is lung adenocarcinoma. In some embodiments, the cancer belongs to the clonal hematopoiesis disorders. In some embodiments, the cancer is a human tumor predisposition syndrome such as, but not limited to, schwannomatosis and Noonan syndrome.

In some embodiments the cancer harbors one or more genetic alterations in the tumor suppressor gene LZTR1. Genetic loss of LZTR1 is found in several cancer types. In some embodiments, the cancer is glioblastoma. In some embodiments, the cancer is hepatocellular cancer. In some embodiments, the cancer is esophagogastric cancer. In some embodiments, the cancer is colorectal carcinoma. In some embodiments, the cancer is breast carcinoma. In some embodiments, the cancer is lung adenocarcinoma. In some embodiments, the cancer belongs to the clonal hematopoiesis disorders. In some embodiments, the cancer is a human tumor predisposition syndrome such as, but not limited to, schwannomatosis and Noonan syndrome. In some embodiments, the genetic loss is due to one or more mutations which result in LZTR1 protein disruptions. In some embodiments, the genetic loss is due to LZTR1 gene deletion.

The LZTR1 gene encodes a protein that functions as a scaffold for ubiquitin ligase complexes, enzymes that tag proteins with ubiquitin molecules leading to the proteins' degradation through the proteasomal system. In some embodiments, the subject matter described herein relates to the oncogenic receptor tyrosine kinases EGFR and AXL, which are substrates of the LZTR1-containing ubiquitin ligase complex. In some embodiments, loss of LZTR1, and disabling the ubiquitin ligase activity, leads to stabilization and accumulation of EGFR and AXL. In some embodiments, EGFR and AXL accumulation drives cancer expansion. In some embodiments, accumulation of EGFR and AXL not only fosters cancer hallmarks but also induces resistance to cancer therapy.

In some embodiments, the subject matter described herein relates to targeted cancer therapy. In some embodiments the cancer therapy includes a combination of inhibitors of the receptor tyrosine kinases. In some embodiments, the tyrosine receptor kinases are EGFR and AXL. In some embodiments the cancer therapy includes a combination of inhibitors of EGFR and AXL. In some embodiments, the EGFR and AXL inhibitor combination is applied to any cancer type. In some embodiments, the cancer is glioblastoma. In some embodiments, the cancer is hepatocellular cancer. In some embodiments, the cancer is esophagogastric cancer. In some embodiments, the cancer is colorectal carcinoma. In some embodiments, the cancer is breast carcinoma. In some embodiments, the cancer is lung adenocarcinoma. In some embodiments, the cancer belongs to the clonal hematopoiesis disorders. In some embodiments, the cancer is a human tumor predisposition syndrome such as, but not limited to, schwannomatosis and Noonan syndrome. In some embodiments, the cancer harbors genetic alterations in the LZTR1 gene.

In some embodiments, the subject matter described herein relates to cancer therapeutics for a selected group of cancer patients. In some embodiments, the cancer patients can be identified by the presence of genetic alterations of the LZTR1 gene. In some embodiments, the cancer patients can be stratified in rational clinical trials for treatment with a combination EGFR and AXL inhibitors.

In certain aspects, the subject matter described herein provides a method of treating cancer in a subject in need thereof, the method comprising administering to the subject a pharmaceutical composition comprising a therapeutically effective amount of at least one Leucine Zipper-like Transcription Regulator 1 (LZTR1) substrate inhibitor, wherein the cancer is associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, a cell from the cancer or a tumor has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL-3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof.

In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the pharmaceutical composition comprises a first RTK inhibitor and a second RTK inhibitor. In some embodiments, both the first and second RTK inhibitors are small molecule inhibitors. In some embodiments, the pharmaceutical composition comprises an EGFR inhibitor and an AXL inhibitor. In some embodiments, the two RTK inhibitors are osimertinib or a pharmaceutically acceptable salt thereof and bemcentinib or a pharmaceutically acceptable salt thereof. In some embodiments, the two RTK inhibitors are afatinib or a pharmaceutically acceptable salt thereof and bemcentinib or a pharmaceutically acceptable salt thereof. In some embodiments, the pharmaceutical composition comprises a first RTK inhibitor and the method further comprising administering a second pharmaceutical composition comprising a second RTK inhibitor, which is different from the first RTK inhibitor. In some embodiments, the pharmaceutical composition comprises an EGFR inhibitor and the second pharmaceutical composition comprises an AXL inhibitor. In some embodiments, the pharmaceutical composition comprises osimertinib or a pharmaceutically acceptable salt thereof and the second pharmaceutical composition comprises bemcentinib or a pharmaceutically acceptable salt thereof. In some embodiments, the pharmaceutical composition comprises afatinib or a pharmaceutically acceptable salt thereof and the second pharmaceutical composition comprises bemcentinib or a pharmaceutically acceptable salt thereof. In some embodiments, the pharmaceutical composition and the second pharmaceutical composition are administered to the subject at the same time. In some embodiments, the pharmaceutical composition and second pharmaceutical composition are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene. In some embodiments, administration of the first and second RTK inhibitors provides a synergistic effect compared to administration of either inhibitor alone. In some embodiments, the subject is a human.

In certain aspects, the subject matter disclosed herein provides a pharmaceutical composition comprising a therapeutically effective amount of an EGFR inhibitor and an AXL inhibitor.

In some embodiments, the EGFR inhibitor comprises osimertinib or a pharmaceutically acceptable salt thereof and the AXL inhibitor comprises bemcentinib or a pharmaceutically acceptable salt thereof. In some embodiments, the EGFR inhibitor comprises afatinib or a pharmaceutically acceptable salt thereof and the AXL inhibitor comprises bemcentinib or a pharmaceutically acceptable salt thereof.

In some embodiments, the pharmaceutical composition further comprises at least one pharmaceutically acceptable excipient, diluent, and/or carrier. In some embodiments, the pharmaceutical composition is for treating or ameliorating the effects of cancer in a subject wherein the cancer is associated with a loss-of-function mutation in a LZTR1 gene. In some embodiments, administration of the EGFR and AXL inhibitors provides a synergistic effect compared to administration of either inhibitor alone.

In certain aspects, the subject matter described herein provides a kit comprising a first pharmaceutical composition comprising a therapeutically effective amount of an EGFR inhibitor and a second pharmaceutical composition comprising a therapeutically effective amount of an AXL inhibitor.

In some embodiments, the EGFR inhibitor comprises osimertinib or a pharmaceutically acceptable salt thereof and the AXL inhibitor comprises bemcentinib or a pharmaceutically acceptable salt thereof. In some embodiments, the EGFR inhibitor comprises afatinib or a pharmaceutically acceptable salt thereof and the AXL inhibitor comprises bemcentinib or a pharmaceutically acceptable salt thereof. In some embodiments, each pharmaceutical composition further comprises at least one pharmaceutically acceptable excipient, diluent, and/or carrier. In some embodiments, the combination therapy is for treating or ameliorating the effects of cancer in a subject wherein the cancer is associated with a loss-of-function mutation in a LZTR1 gene. In some embodiments, administration of the combination therapy provides a synergistic effect compared to administration of either pharmaceutical composition alone. In some embodiments, the first and second pharmaceutical compositions are packaged together with instructions for their use.

In certain aspects, the subject matter described herein provides a method of decreasing growth of a solid tumor in a subject in need thereof, the method comprising administering to the subject a pharmaceutical composition comprising a therapeutically effective amount of at least one Leucine Zipper-like Transcription Regulator 1 (LZTR1) substrate inhibitor, wherein the composition decreases the size of the solid tumor, and wherein the tumor is associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, a cell from the cancer or a tumor has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof. In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the pharmaceutical composition comprises a first RTK inhibitor and a second RTK inhibitor. In some embodiments, both the first and second RTK inhibitors are small molecule inhibitors. In some embodiments, the pharmaceutical composition comprises an EGFR inhibitor and an AXL inhibitor. In some embodiments, the two RTK inhibitors are osimertinib or a pharmaceutically acceptable salt thereof and bemcentinib or a pharmaceutically acceptable salt thereof. In some embodiments, the two RTK inhibitors are afatinib or a pharmaceutically acceptable salt thereof and bemcentinib or a pharmaceutically acceptable salt thereof. In some embodiments, the pharmaceutical composition comprises a first RTK inhibitor and the method further comprises administering a second pharmaceutical composition comprising a second RTK inhibitor, which is different from the first RTK inhibitor. In some embodiments, the pharmaceutical composition comprises an EGFR inhibitor and the second pharmaceutical composition comprises an AXL inhibitor. In some embodiments, the pharmaceutical composition comprises osimertinib or a pharmaceutically acceptable salt thereof and the second pharmaceutical composition comprises bemcentinib or a pharmaceutically acceptable salt thereof. In some embodiments, the pharmaceutical composition comprises afatinib or a pharmaceutically acceptable salt thereof and the second pharmaceutical composition comprises bemcentinib or a pharmaceutically acceptable salt thereof. In some embodiments, the pharmaceutical composition and the second pharmaceutical composition are administered to the subject at the same time. In some embodiments, the pharmaceutical composition and the second pharmaceutical composition are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene.

In some embodiments, administration of the first and second RTK inhibitors provides a synergistic effect compared to administration of either inhibitor alone. In some embodiments, the subject is a human.

In certain aspects, the subject matter described herein provides a method of effecting cancer cell death, the method comprising contacting the cancer cell with an effective amount of at least one Leucine Zipper-like Transcription Regulator 1 (LZTR1) substrate inhibitor, wherein the cancer cell is from a cancer associated with a loss-of-function mutation in a LZTR1 gene.

In some embodiments, the cancer is associated with a loss-of-function mutation in the LZTR1 gene if one or more cells of the subject comprise the loss-of-function mutation in the LZTR1 gene. In some embodiments, the cancer cell has the loss-of-function mutation in the LZTR1 gene.

In some embodiments, the mutation is a somatic mutation. In some embodiments, the mutation is a germline mutation. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I. In some embodiments, the mutation is located in a Kelch domain of the LZTR1 gene. In some embodiments, the mutation is H71R, P115L, S122L, R170Q, L187R, M202R, or M400R. In some embodiments, the mutation is located in a CUL3-interacting domain of the LZTR1 gene. In some embodiments, the mutation is located in a substrate-interacting domain of the LZTR1 gene.

In some embodiments, the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorders or tumor predisposition syndromes.

In some embodiments, the cancer is associated with higher levels of at least one LZTR1 substrate compared to a sample of non-cancerous tissue. In some embodiments, the LZTR1 substrate with higher levels is a Receptor Tyrosine Kinase (RTK). In some embodiments, the RTK is Epidermal Growth Factor Receptor (EGFR). In some embodiments, the RTK is AXL. In some embodiments, the LZTR1 substrate with higher levels is RIT1.

In some embodiments, the inhibitor comprises a small molecule inhibitor of a LZTR1 substrate, an antibody that specifically binds to a LZTR1 substrate, or a fragment thereof, an antisense RNA or antisense DNA that decreases expression of a polypeptide of a LZTR1 substrate; a siRNA that specifically targets a LZTR1 substrate mRNA, a sgRNA that specifically targets a nucleotide sequence encoding a LZTR1 substrate, or a combination thereof. In some embodiments, the LZTR1 substrate inhibitor is a Receptor Tyrosine Kinase (RTK) inhibitor. In some embodiments, the RTK inhibitor is an Epidermal Growth Factor Receptor (EGFR) inhibitor. In some embodiments, the RTK inhibitor is an AXL inhibitor. In some embodiments, the LZTR1 substrate inhibitor is a RIT1 inhibitor.

In some embodiments, the inhibitor comprises a small molecule inhibitor. In some embodiments, the inhibitor comprises a small molecule inhibitor of a LZTR1 substrate, wherein the small molecule inhibitor is afatinib or a pharmaceutically acceptable salt thereof, osimertinib or a pharmaceutically acceptable salt thereof, or bemcentinib or a pharmaceutically acceptable salt thereof.

In some embodiments, the at least one LZTR1 substrate inhibitor comprises a first RTK inhibitor and a second RTK inhibitor. In some embodiments, both the first and second RTK inhibitors are small molecule inhibitors. In some embodiments, the first and second RTK inhibitors comprise an EGFR inhibitor and an AXL inhibitor, respectively. In some embodiments, the first and the second RTK inhibitors are osimertinib or a pharmaceutically acceptable salt thereof and bemcentinib or a pharmaceutically acceptable salt thereof. In some embodiments, the first and the second RTK inhibitors are afatinib or a pharmaceutically acceptable salt thereof and bemcentinib or a pharmaceutically acceptable salt thereof.

In some embodiments, the at least one LZRT1 substrate inhibitor comprises a first RTK inhibitor and the method further comprises administering a second pharmaceutical composition comprising a second RTK inhibitor, which is different from the first RTK inhibitor. In some embodiments, the least one LZRT1 substrate inhibitor an EGFR inhibitor and the second pharmaceutical composition comprises an AXL inhibitor. In some embodiments, the least one LZRT1 substrate inhibitor comprises osimertinib or a pharmaceutically acceptable salt thereof and the second pharmaceutical composition comprises bemcentinib or a pharmaceutically acceptable salt thereof. In some embodiments, the least one LZRT1 substrate inhibitor comprises afatinib or a pharmaceutically acceptable salt thereof and the second pharmaceutical composition comprises bemcentinib or a pharmaceutically acceptable salt thereof. In some embodiments, the first and second LZRT1 substrate inhibitors are administered to the subject at the same time. In some embodiments, the first and second LZRT1 substrate inhibitor are administered to the subject at different times.

In some embodiments, the cancer is associated with a loss-of-function mutation in a Cdkn2A gene. In some embodiments, the cancer is associated with a loss-of-function mutation in the Cdkn2A gene if one or more cells of the subject comprise the loss-of-function mutation in the Cdkn2A gene. In some embodiments, a cell from the cancer or a tumor has the loss-of-function mutation in the Cdkn2A gene.

In certain aspects, the subject matter described herein provides a method for detecting the presence of a LZTR1 protein in a subject, the method comprising: (a) obtaining a biological sample from the subject; and (b) detecting whether or not there is a LZTR1 protein present in the subject.

In some embodiments, the detecting comprises measuring LZRT1 protein levels by an antibody directed to the LZRT1 protein, Western blot using an antibody directed to the LZRT1 protein, ELISA using an antibody directed to the LZRT1 protein, mass spectroscopy, or a combination thereof. In some embodiments, the method further comprises administering to the subject a pharmaceutical composition comprising a therapeutically effective amount of at least one RTK inhibitor if the LZTR1 protein is not present in the sample.

In certain aspects, the subject matter described herein provides a method for detecting the presence of a mutant LZTR1 protein in a subject, the method comprising: (a) obtaining a biological sample from the human subject; and (b) detecting whether or not there is a nucleic acid sequence encoding a mutant LZTR1 protein in the subject.

In some embodiments, the detecting comprises using hybridization, amplification, or sequencing techniques to detect the mutant LZTR1 protein. In some embodiments, the LZTR1 mutation comprises one or more nucleotide insertion, deletion, or substitution mutations in the LZTR1 gene or any combination thereof. In some embodiments, the LZTR1 mutation comprises a deletion of at least a portion of the LZTR1 gene. In some embodiments, the LZTR1 mutation is located in a BTB-Back domain of the LZTR1 gene. In some embodiments, the LZTR1 mutation is V456G, P520L, L591R, R688C, R810W, or S813I. In some embodiments, the LZTR1 mutation is located in a Kelch domain of the LZTR1 gene. In some embodiments, the mutation is H71R, P115L, S122L, R170Q, L187R, M202R, or M400R. In some embodiments, the LZTR1 mutation is located in a CUL-3-interacting domain of the LZTR1 gene. In some embodiments, the LZTR1 mutation is located in a substrate-interacting domain of the LZTR1 gene. In some embodiments, the method further comprises administering to the subject a pharmaceutical composition comprising a therapeutically effective amount of at least one RTK inhibitor if a nucleic acid sequence encoding a mutant LZTR1 protein is present in the sample.

In certain aspects, the subject matter described herein provides a diagnostic kit for determining whether a sample from a subject exhibits a presence of a nucleic acid encoding a mutant LZTR1 protein, the kit comprising at least one oligonucleotide that specifically hybridizes to the nucleic acid encoding the mutant LZTR1.

In some embodiments, the at least one oligonucleotide comprises a set of nucleic acid primers or in situ hybridization probes. In some embodiments, the primers prime a polymerase reaction only when a mutant LZTR1 protein is present.

In some embodiments, the LZTR1 mutation comprises one or more nucleotide insertion, deletion, or substitution mutations in the LZTR1 gene or any combination thereof. In some embodiments, the LZTR1 mutation comprises a deletion of at least a portion of the LZTR1 gene. In some embodiments, the LZTR1 mutation is located in a BTB-Back domain of the LZTR1 gene. In some embodiments, the LZTR1 mutation is V456G, P520L, L591R, R688C, R810W, or S813I. In some embodiments, the LZTR1 mutation is located in a Kelch domain of the LZTR1 gene. In some embodiments, the mutation is H71R, P115L, S122L, R170Q, L187R, M202R, or M400R. In some embodiments, the LZTR1 mutation is located in a CUL-3-interacting domain of the LZTR1 gene. In some embodiments, the LZTR1 mutation is located in a substrate-interacting domain of the LZTR1 gene. In some embodiments, the determining comprises gene sequencing, selective hybridization, selective amplification, gene expression analysis, or a combination thereof.

### Ubiquitination and the Role of Leucine Zipper-Like Transcription Regulator 1 (LZTR1)

Ubiquitylation is a key post-translational protein modification that regulates protein stability, cell proliferation and differentiation. An imbalance in ubiquitination-mediated protein degradation can lead to the development of tumors and cancers such as glioblastoma, prostate cancer, lung cancer, liver cancer, etc. Thus, the ubiquitination process, which target proteins for degradation, is a complicated and tightly regulated process. Under the action of the ubiquitin-conjugating enzyme E2, activated ubiquitin is transferred to a specific substrate (protein targeted for degradation) along with an E3 ubiquitin ligase. Substrate recognition for ubiquitin ligation is determined by E3 ubiquitin ligases. One of the best known E3 ligase family is cullin (CUL)-RING E3 ubiquitin ligase, which consists of a molecular scaffold connecting a substrate-specific adaptor protein to a catalytic component comprising a RING finger domain and an E2 ubiquitin-conjugating enzyme. E3 adaptors of CUL3 include speckle-type protein (SPOP), Kelch repeat and BTB domain-containing protein 8 (KBTBD8) and Kelch-like ECH-associated protein 1 (KEAP1). These adaptors are composed of a similar structure called broad-complex, tramtrack and bric-a-brac (BTB) domain, which combines the substrate receptor and adaptor functions into the CUL3-RBX1 E3 ubiquitin ligase complex.

LZTR1 is also a member of the BTB-Kelch superfamily proteins. It is the substrate-specific adaptor for CUL3 ubiquitin ligase complex. LZTR1 was initially identified as a transcriptional regulator due to its weak homology to known members of the basic leucine zipper-like family. BTB-Kelch superfamily protein members generally interact with actin filaments and play important roles in transcriptional regulation and protein ubiquitination. However, LZTR1 shows no interaction with actin but is localized on the Golgi complex. Loss of function LZTR1 mutations ultimately lead to accumulation of one or more LZRT1 substrates in the cell due to abrogated degradation. Mutations in the LZTR1 gene occur in 4.4% of those with GBM and 24.4% of those with schwannomatosis. These diseases associated with abnormal function of RAS proteins, which is consistent with the fact that the RAS superfamily, including KRAS, NRAS, RAS-like without CAAX1 (RIT1) and RAF1, are substrates of LZTR1 for ubiquitination. Disease-associated LZTR1 mutations lead to loss of LZTR1 ubiquitination capability and ultimately to excessive activation of RAS/MAPK signaling.

### LZTR1 Mutations

The primary structure of the LZTR1 protein includes six Kelch motifs at the N-terminus and two C-terminal BTB-BACK domains. The Kelch domains selectively recruit substrates (target protein for ubiquitination). The BACK domains mediate dimerization and the binding of LZTR1 to CUL3. The majority of glioblastoma-related LZTR1 mutations are clustered in the Kelch domains. These mutant proteins are defective in LZTR1-mediated substrate binding, thus preventing the formation of substrate-LZTR1-CUL3 complexes and the efficient ubiquitination and degradation of the substrate. This cascade ultimately results in excessive activation and/or accumulation of the substrate. Examples of such mutations include, but are not limited to, Trp105Arg, Asp139Ala, Asn143Thr, Gly195Ser, Arg198Gly, Gly248Arg, Arg284Ser, Thr288Ile, and G404Glu in the Kelch domains. An example of a LZTR1 mutation in the BTB-BACK domains is Arg810Trp. Most mutations in the LZTR1 BACK domains exhibit reduced interaction with CUL3.

Schwannomatosis-associated LZTR1 mutations are more evenly distributed across all domains These mutations can result in failure to bind with CUL3. Example of schwannomatosis-associated LZTR1 mutations in the Kelch domains include, but are not limited to, His71Arg, Pro115Leu, Ser122Leu, Arg170Gln, Leu187Leu, Met202Arg, Arg284Cys, Gly285Arg, Met400Arg, and Gly404Arg. Example of schwannomatosis-associated LZTR1 mutations in the BTB-BACK domains include, but are not limited to, Val456Gly, Arg466Gln (Trp), Pro520Leu, Met665Lys, Arg688His(Cys), Leu812Pro, and Ser813Leu.

LZTR1 mutations in GBM include 4.4% non-synonymous mutations and 22.4% focal deletions in the coding sequence. RIT1 is an important pathogenic factor of GBM. RIT1 participates in the activation of the MAPK/ERK signaling pathway and plays crucial roles in many physiological processes. Endogenous RIT1 interacts with LZTR1 and may mediate the effects of mutant LZTR1 in GBM. 9 out of 10 LZTR1 mutations occur in the LZTR1 Kelch domains and greatly impair the RIT1-LZTR1 interaction. Therefore, LZTR1 suppresses the MAPK/ERK signaling pathway by degrading RIT1 to inhibit proliferation and migration of GBM cells; however, GBM-associated LZTR1 mutations impair this function.

Schwannomatosis is a hereditary disease characterized by schwannomas in the spinal and peripheral nerves as well as benign tumors throughout the nervous system. Germline mutations in the LZTR1 gene occur in 41 out of 168 sporadic patients with schwannomatosis (24.4%). Schwannomatosis-associated LZTR1 mutations are uniformly located in almost every LZTR1 domain.

In some embodiments, the subject matter described herein relates to mutant LZTR1 protein having at least one mutation that leads to loss of LZTR1 function. In some embodiments, the mutation is in any LZTR1 domain. In some embodiments, mutant LZTR1 has multiple mutations in different domains. In some embodiments, the mutation comprises one or more nucleotide insertion, deletion, or substitution mutations in the LZTR1 gene or any combination thereof. In some embodiments, the mutation comprises a deletion of at least a portion of the LZTR1 gene. In some embodiments, the mutation is located in a BTB-Back domain of the LZTR1 gene. In some embodiments, the mutation is V456G, P520L, L591R, R688C, R810W, or S813I in the BTB-Back domain. In some embodiments, the mutation is located in a Kelch domain of the LZTR1 gene. In some embodiments, the mutation is H71R, P115L, S122L, R170Q, L187R, M202R, or M400R in the Kelch domain. In some embodiments, wherein the mutation is located in a CUL-3-interacting domain of the LZTR1 gene. In some embodiments, the mutation is located in a substrate-interacting domain of the LZTR1 gene. In some embodiments, mutant LZTR1 fails to ubiquitinate a substrate. In some embodiments, the substrate is a RTK. In some embodiments, the substrate is EGFR. In some embodiments, the substrate is AXL. In some embodiments, the substrate is RIT1.

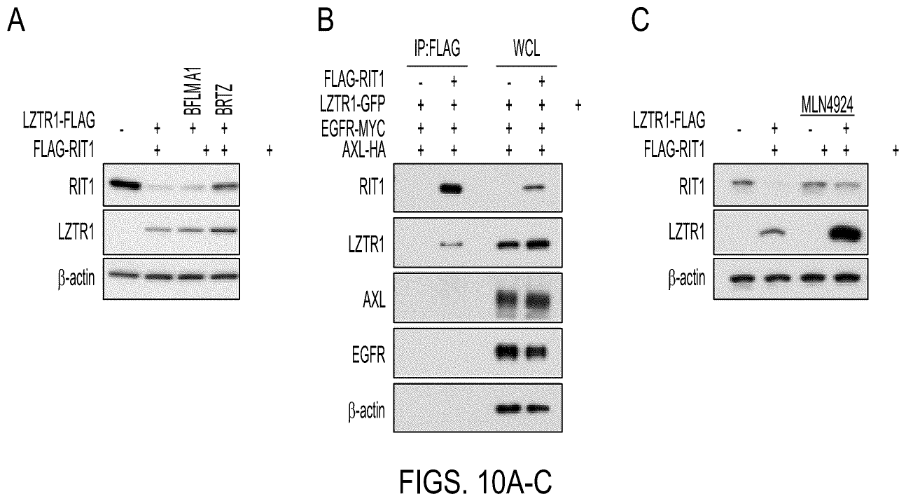

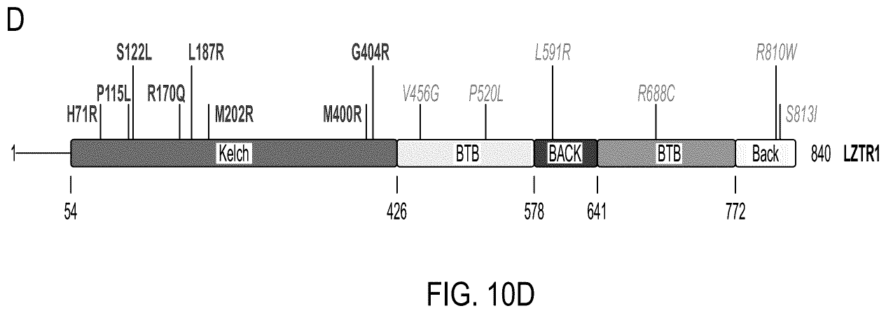

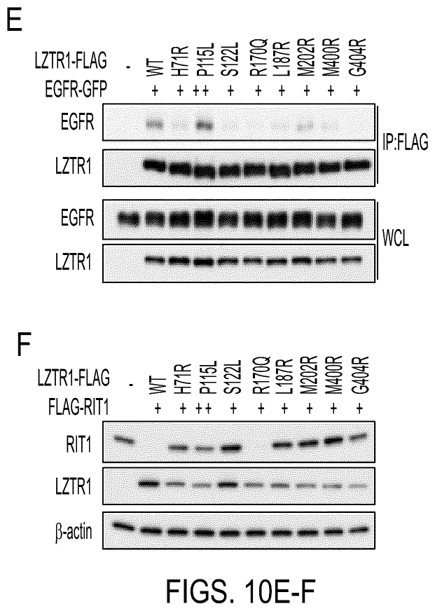

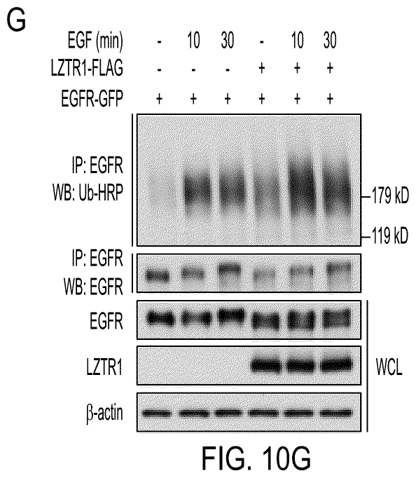

The amino acid sequence of human LZTR1 can be accessed at uniprot.org using accession number Q8N653, the amino acid sequence is hereby incorporated by reference and can be easily accessed by a person of skill in the art. Uniprot also provides the domains of LZTR1. In some embodiments, the Kelch domain comprises amino acids 54-425 as shown in FIG. 10D. In some embodiments, the BTB-Back domain comprises amino acids 426-840, with BTB and Back domains as shown in FIG. 10D.

Receptor Tyrosine Kinases and their Inhibitors

Receptor tyrosine kinases (RTK s) are major components of many signal transduction pathways that mediate cell-to-cell communication. RTK s are single-pass transmembrane receptors, which bind polypeptide ligands, mainly growth factors, and play key roles in processes such as cellular growth, differentiation, metabolism and motility. RTKs are activated through ligand-induced oligomerization of the receptor, typically dimerization, which brings together the cytoplasmic domains of the receptor, which contain the tyrosine kinase domains. This facilitates autophosphorylation of tyrosine residues in the kinase activation loop, inducing conformational changes that ultimately stabilize the active state of the kinase. These phosphotyrosine residues serve as recruitment sites for a various down-stream signaling proteins.

The epidermal growth factor receptor (EGFR) is a receptor tyrosine kinase that is activated by binding of its ligand, the epidermal growth factor (EGF). EGFR is a single chain transmembrane glycoprotein consisting of an extracellular ligand-binding ectodomain, a transmembrane domain, a short juxtamembrane section, a tyrosine kinase domain and a tyrosine-containing C-terminal tail. Binding of its soluble EGF ligand to the ectodomain of the receptor promotes receptor dimer formation and initiation of downstream signaling cascades. Phosphotyrosine residues activate, either directly or through adaptor proteins, downstream components of various signaling pathways including Ras/MAPK, PLCγ1/PKC, PI(3)kinase/Akt, and STAT pathways. Activated EGFR stimulates cell proliferation, survival, differentiation and migration. Tight regulatory control of these EGFR signaling pathways is paramount to avoid tumor development. The active EGFR signal can be immediately quenched through dephosphorylation, internalization of the activated receptors, and ubiquitination and degradation of the activated receptors. Activated, tyrosine phosphorylated EGFR can bind the E3 ubiquitin ligase Cbl, which is capable of ubiquitinylating EGFR leading to lysosomal degradation of the receptor.

AXL is a member of the TAM family. AXL's high-affinity ligand is growth arrest-specific protein 6 (GAS6). The Gas6/AXL signaling pathway plays a role in tumor cell growth, cancer, metastasis, invasion, epithelial-mesenchymal transition (EMT). Different therapeutic agents targeting AXL have been developed, for example small molecule inhibitors, monoclonal antibodies (mAbs), nucleotide aptamers, soluble receptors, and several natural compounds. The AXL gene is located at chromosome 19q13.2 and was first identified in patients with chronic myeloid leukaemia (CM L). The extracellular domain of AXL consists of two immunoglobulin (Ig)-like repeats and two fibronectin type III (Fro III)-like repeats that resemble neural cell adhesion molecules (NCAMs). The Ig motifs play a role in the binding of AXL with its ligand Gas6. The intracellular domain is involved in autophosphorylation and AXL's subsequent kinase activity. AXL is expressed in monocytes, platelets, endothelial cells, hippocampus, cerebellum, heart, and liver. AXL regulates cell survival, the non-inflammatory clearance of apoptotic cells by phagocytic cells, natural killer cell differentiation, platelet aggregation among others. Binding of Axl to Gas6 also induces the ubiquitination of Axl and the interaction of Axl with the ubiquitin ligase c-Cbl. This posttranslational modification is required for efficient AXL degradation in the lysosome.

Osimertinib is currently the only FDA- and EMA-approved third-generation small-molecule epidermal growth factor receptor (EGFR) tyrosine kinase inhibitor. It was initially indicated for second-line treatment of patients with metastatic EGFR T790M mutation-positive non-small cell lung cancer (NSCLC). Osimertinib selectively targets the intracellular tyrosine kinase domain of EGFR. Osimertinib inhibits overactive downstream signals of the EGFR signaling pathway, thus, inhibiting proliferation, differentiation, and survival of the cancerous cells are suppressed. Osimertinib has the following structure:

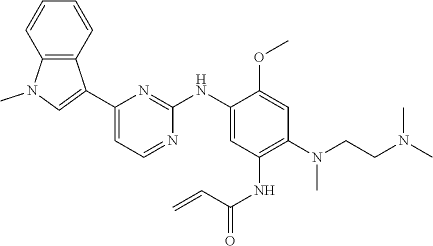

In some embodiments, the methods, compositions, and kits described herein comprise osimertinib. In some embodiments, the methods, compositions, and kits described herein comprise osimertinib or pharmaceutically acceptable salts thereof.

Afatinib is a kinase inhibitor used as monotherapy for the first-line treatment of Epidermal Growth Factor Receptor (EGFR) tyrosine kinase inhibitor in naive adult patients with locally advanced or metastatic non-small cell lung cancer (NSCLC). Patients usually have tumors with non-resistant EGFR mutations. Afatinib covalently binds to the kinase domains of EGFR, resulting in irreversible inhibition of tyrosine kinase autophosphorylation. Afatinib has the following structure:

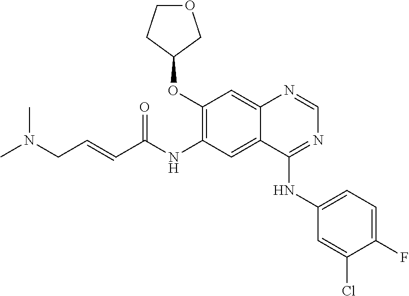 [SMILES: CN(C)C/C=C/C(=O)Nc1cc2c(Nc3ccc(F)c(Cl)c3)ncnc2cc1O[C@H]1CCOC1]

In some embodiments, the methods, compositions, and kits described herein comprise afatinib. In some embodiments, the methods, compositions, and kits described herein comprise afatinib or pharmaceutically acceptable salts thereof.

Bemcentinib had is a small molecule inhibitor of AXL. Bemcentinib targets the intracellular catalytic kinase domain of the AXL receptor tyrosine kinase. Upon binding, it inhibits its kinase activity. Bemcentinib also enhances sensitivity to various therapies including chemotherapy, immunotherapy and several targeted therapeutics. Bemcentinib has the following structure:

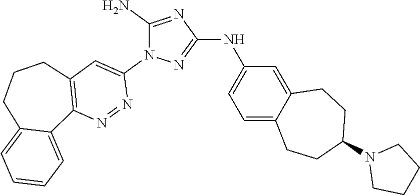 [SMILES: Nc1nc(Nc2ccc3c(c2)CC[C@@H](N2CCCC2)CC3)nn1-c1cc2c(nn1)-c1ccccc1CCC2]

In some embodiments, the methods, compositions, and kits described herein comprise bemcentinib. In some embodiments, the methods, compositions, and kits described herein comprise bemcentinib or pharmaceutically acceptable salts thereof.

### Cancer Treatment

In some embodiments, the subject matter described herein provides a method of decreasing the growth of a solid tumor in a subject in need thereof. In some embodiments, the tumor is associated with, but not limited to, glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes. In one embodiment, the method comprises detecting the presence of a mutant LZTR1 protein in a sample obtained from a subject. In some embodiments, the sample is incubated with an agent that binds to a mutant LZTR1 protein or a nucleic acid encoding mutant LZTR1 protein, such as an antibody, a probe, a nucleic acid primer, and the like.

In some embodiments, the subject matter described herein relates to treatment of mutant LZRT1 protein-associated solid tumor or cancer. In some embodiments, a tumor or cancer is associated with mutant LZRT1 if the subject has a mutation in LZRT1. In some embodiments, a tumor or cancer is associated with mutant LZRT1 if one or more cells of the tumor or cancer of the subject comprises a mutation in LZRT1. In some embodiments, the solid tumor or cancer is associated with accumulation of one or more substrates of LZRT1 protein. In some embodiments, the treatment comprises administering a pharmaceutical composition to a subject in need thereof comprising a therapeutically effective amount of an agent that decreases the expression levels or activity of a substrate of a LZRT1 protein. In some embodiments, the treatments, the substrate is a RTK. In some embodiments, the RTK is EGFR. In some embodiments, the RTK is AXL. In some embodiments, the substrate is RIT1. In some embodiments, the treatment comprises administering to the subject an effective amount of a RTK inhibitor, wherein the inhibitor decreases the size of the solid tumor. In some embodiments, the inhibitor is an EGFR inhibitor. In some embodiments, the RTK inhibitor is an AXL inhibitor. In some embodiments, the inhibitor is afatinib, osimertinib, bemcentinib, or any combination thereof. In some embodiments, the pharmaceutical composition comprises two RTK inhibitors. In some embodiments, the two inhibitors are afatinib and bemcentinib. In some embodiments, the two inhibitors are osimertinib and bemcentinib.

In some embodiments, the subject matter disclosed herein also provides a method for treating or preventing a mutant LZTR1 protein-associated cancer in a subject. In one embodiment, the mutant LZTR1 protein-associated cancer comprises glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes. In one embodiment, the method comprises detecting the presence of a mutant LZTR1 protein in a sample obtained from a subject, the presence of the mutant protein being indicative of a mutant LZTR1 protein-associated cancer, and, administering to the subject in need a therapeutic treatment against the mutant LZTR1 protein-associated cancer. In some embodiments, the sample is incubated with an agent that binds to a mutant LZTR1 protein, such as an antibody, a probe, a nucleic acid primer, and the like.

In some embodiments, the subject matter described herein also provides a method for decreasing in a subject in need thereof the expression level or activity of a substrate of a LZTR1 protein. In some embodiments the LZTR1 protein is a mutant LZTR1 protein. In some embodiments, the substrate is a RTK. In some embodiments, the RTK is EGFR. In some embodiments, the RTK is AXL. In some embodiments, the substrate is RIT1. In some embodiments, the method comprises obtaining a biological sample from the subject. In some embodiments, the sample is incubated with an agent that binds to a mutant LZTR1 protein, such as an antibody, a probe, a nucleic acid primer, and the like. In some embodiments, the method comprises administering to the subject a therapeutic amount of a composition comprising an admixture of a pharmaceutically acceptable excipient, diluent, and/or carrier and an inhibitor of a substrate of the LZTR1 protein. In another embodiment, the method further comprises determining the mutant LZTR1 protein expression level or activity. In another embodiment, the method further comprises determining the expression level or activity of a substrate of the LZTR1 protein. In another embodiment, the method further comprises detecting whether the level or activity of the substrate of the LZTR1 protein is decreased as compared to the expression level or activity of the substrate of the LZTR1 protein prior to administration of the composition, thereby decreasing the expression level or activity of the substrate of the LZTR1 protein.

In some embodiments, the mutation of the mutant LZTR1 protein comprises one or more nucleotide insertion, deletion, or substitution mutations in the LZTR1 gene or any combination thereof. In some embodiments, the mutation of the mutant LZTR1 protein comprises a deletion of at least a portion of the LZTR1 gene. In some embodiments, the mutation of the mutant LZTR1 protein is located in a BTB-Back domain of the LZTR1 gene. In some embodiments, the mutation is V456G, P520L, L591R, R688C, R810W, or S813I in the BTB-Back domain. In some embodiments, the mutation of the mutant LZTR1 protein is located in a Kelch domain of the LZTR1 gene. In some embodiments, the mutation is H71R, P115L, S122L, R170Q, L187R, M202R, or M400R in the Kelch domain. In some embodiments, the mutation of the mutant LZTR1 protein is located in a CUL-3-interacting domain of the LZTR1 gene. In some embodiments, the mutation of the mutant LZTR1 protein is located in a substrate-interacting domain of the LZTR1 gene.

The administering step in each of the methods described herein can comprise administration of a pharmaceutical composition, such as a RTK inhibitor (for example, a small molecule that specifically binds to a RTK such as afatinib, osimertinib, bemcentinib, or any combination thereof; an antisense RNA or antisense DNA that decreases expression of a substrate of a LZTR1 protein; a siRNA that specifically targets a gene encoding a substrate of a LZTR1 protein; or a combination thereof). In another embodiment, administration of the pharmaceutical composition decreases the size of the solid tumor. In some embodiments, the solid tumor is associated with glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes.

In another embodiment, the pharmaceutical composition to be administered comprises an siRNA directed to a human nucleic acid sequence comprising a substrate of a LZTR1 protein. In a further embodiment, the pharmaceutical composition to be administered comprises an antibody or binding fragment thereof, which is directed against a substrate of a LZTR1 protein. In some embodiments, the pharmaceutical composition to be administered comprises a small molecule that specifically binds to the LZTR1 substrate. In some embodiments, the substrate is an RTK. In some embodiments, the RTK is EGFR. In some embodiments, the RTK is AXL. In some embodiments, the small molecule is afatinib, osimertinib, bemcentinib, or any combination thereof.

In one embodiment, the subject matter described herein provides for the detection of a chromosomal rearrangement at given chromosomal coordinates. In some embodiments, the chromosomal rearrangement affects the LZTR1 gene. In some embodiments, the chromosomal rearrangement is in human chromosome 22. In some embodiments, the chromosomal coordinates are 22q11.21. In some embodiments, these chromosomal coordinates encompass a gene encoding the LZTR1 protein. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, ELISA, or other antibody detection methods. In some embodiments, a mutation in LZTR1 can be determined at the level of the DNA, RNA, or polypeptide. In some embodiments, the detections can be determined by performing a hybridization assay, a sequencing assay, an allele-specific amplification assay, a microsequencing assay, a melting curve analysis, a denaturing high performance liquid chromatography (DHPLC) assay (for example, see J ones et al, (2000) Hum Genet., 106(6):663-8), or a combination thereof. In one embodiment, the detection is performed by sequencing all or part of a nucleic acid encoding the LZTR1 protein or by selective hybridization or amplification of all or part of a nucleic acid encoding the LZTR1 protein.

In some embodiments, the subject matter described herein provides for a method of detecting a chromosomal alteration in a subject afflicted with a mutant LZTR1 protein-associated cancer. In some embodiments, the chromosomal alteration is a chromosomal translocation. An alteration in a chromosome region occupied by the LZTR1 gene can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or noncoding portion of the gene locus, such as from two residues up to the entire gene or locus.

Deletions can affect smaller regions, such as different domains (introns) or repeated sequences or fragments of less than about 50 consecutive base pairs, although larger deletions can occur as well. Rearrangement includes inversion of sequences. The alteration in a chromosome region occupied by the LZTR1 gene, can result in amino acid substitutions, RNA splicing or processing, product instability, the creation of stop codons, production of oncogenic fusion proteins, frame-shift mutations, and/or truncated polypeptide production. The alteration can result in the production of a mutant LZTR1 protein with altered function, stability, targeting or structure. The alteration can also cause a reduction in, or even elimination of protein expression. This alteration can be determined at the level of the DNA, RNA, or polypeptide. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, ELISA, or other antibody detection methods.

In some embodiments, the subject matter described herein provides a method for treating a mutant LZTR1-associated cancer in a subject in need thereof. In one embodiment, the method comprises obtaining a sample from the subject to determine the level of expression of the LZTR1 protein in the subject. In one embodiment, the method comprises obtaining a sample from the subject to determine the level of expression or activity of a substrate of the LZTR1 protein in the subject. In some embodiments, the sample is incubated with an agent that binds to a mutant LZTR1 protein or to a substrate of the LZTR1 protein, such as an antibody, a probe, a nucleic acid primer, and the like. In another embodiment, the detection or determination comprises nucleic acid sequencing, selective hybridization, selective amplification, gene expression analysis, or a combination thereof. In another embodiment, the detection or determination comprises protein expression analysis, for example by western blot analysis, ELISA, or other antibody detection methods.

In some embodiments, the method further comprises assessing whether to administer an inhibitor of a substrate of the LZTR1 based on the expression pattern of the mutant LZTR1 or its substrates in the subject's sample. In further embodiments, the method comprises administering an inhibitor of a substrate of the LZTR1 to the subject. In one embodiment, the mutant LZTR1-associated cancer comprises an epithelial cancer. In one embodiment, the mutant LZTR1-associated cancer comprises glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes.

In one embodiment, the subject matter described herein provides for a method of detecting the presence of altered RNA expression encoding a mutant LZTR1 protein in a subject, for example one afflicted with a mutant LZTR1 protein-associated cancer. In another embodiment, the invention provides for a method of detecting the presence of a mutant LZTR1 protein in a subject. In some embodiments, the method comprises obtaining a sample from the subject to determine whether the subject expresses a mutant LZTR1 protein. In some embodiments, the sample is incubated with an agent that binds to a mutant LZTR1 protein, such as an antibody, a probe, a nucleic acid primer, and the like. In other embodiments, the detection or determination comprises nucleic acid sequencing, selective hybridization, selective amplification, gene expression analysis, or a combination thereof. In another embodiment, the detection or determination comprises protein expression analysis, for example by western blot analysis, ELISA, or other antibody detection methods. In some embodiments, the method further comprises assessing whether to administer an inhibitor of a substrate of the LZTR1 protein based on the expression pattern of the mutant LZTR1 and/or its substrate in the subject. Altered RNA expression includes the presence of an altered RNA sequence, the presence of an altered RNA splicing or processing, or the presence of an altered quantity of RNA. These can be detected by various techniques known in the art, including sequencing all or part of the RNA or by selective hybridization or selective amplification of all or part of the RNA. Altered polypeptide expression includes the presence of an altered polypeptide sequence, the presence of an altered quantity of polypeptide, or the presence of an altered tissue distribution. These can be detected by various techniques known in the art, including by sequencing and/or binding to specific ligands (such as antibodies and hybridization probes). In one embodiment, the detecting comprises using a northern blot; real time PCR and primers; a ribonuclease protection assay; a hybridization, amplification, or sequencing technique to detect a nucleotide sequence encoding a mutant LZTR1 protein; or a combination thereof.

Various techniques known in the art can be used to detect or quantify altered gene or RNA expression or nucleic acid sequences, which include, but are not limited to, hybridization, sequencing, amplification, and/or binding to specific ligands (such as antibodies and hybridization probes). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some of these approaches (such as SSCA and constant gradient gel electrophoresis (CGGE)) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Hybridization. Hybridization detection methods are based on the formation of specific hybrids between complementary nucleic acid sequences that serve to detect nucleic acid sequence alteration(s). A detection technique involves the use of a nucleic acid probe specific for a wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. For example, a sample from the subject can be contacted with a nucleic acid probe specific for a gene encoding a wild-type or mutant LZTR1 protein, and the formation of a hybrid can be subsequently assessed. In one embodiment, the method comprises contacting simultaneously the sample with a set of probes that are specific for a nucleic acid encoding a wild-type or mutant LZTR1 protein. Also, various samples from various subjects can be investigated in parallel.

In some embodiments, a probe can be a polynucleotide sequence which is complementary to and specifically hybridizes with a, or a target portion of a, gene or RNA corresponding to a nucleic acid encoding a wild-type of a mutant LZTR1 protein. Useful probes are those that are complementary to the gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a gene or RNA that encodes a wild-type or mutant LZTR1 protein.

The sequence of the probes can be derived from the sequences coding a wild-type of mutant LZTR1 protein known in the field. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3rd Ed.), Vols. 1-3, Cold Spring Harbor Laboratory, 1989; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part I. Theory and Nucleic Acid Preparation, Tijssen, ed. Elsevier, N.Y., 1993.

Sequencing. Sequencing can be carried out using techniques well known in the art, using automatic sequencers. The sequencing can be performed on a nucleic acid encoding the complete LZTR1 protein or on specific domains thereof.

Amplification. Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. For example, nucleic acid primers useful for amplifying sequences corresponding to a wild-type or mutant LZTR1 protein are able to specifically hybridize with a portion of the gene locus that flanks a target region of the locus. In certain subjects, the presence of a mutated LZRT1 protein corresponds to a subject with a mutated LZTR1-associated cancer. Non-limiting amplification methods include, e.g., polymerase chain reaction, PCR (PCR Protocols, A Guide To Methods And Applications, ed. Innis, Academic Press, N.Y., 1990 and PCR Strategies, 1995, ed. Innis, Academic Press, Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). A II the references stated above are incorporated herein by reference in their entireties.

Specific Ligand Binding. As discussed herein, a nucleic acid encoding a wild-type or mutant LZTR1 protein or expression of a wild-type or mutant LZTR1 protein, can also be detected by screening for alteration(s) in a sequence or expression level of a polypeptide encoded by the same. Different types of ligands can be used, such as specific antibodies. In one embodiment, the sample is contacted with an antibody specific for a polypeptide encoded by a wild-type or mutant LZTR1 nucleic acid and the formation of an immune complex is subsequently determined. Various methods for detecting an immune complex can be used, such as ELISA, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a wild-type or mutant LZTR1 protein can be an antibody that selectively binds such a protein. In one embodiment, the antibody is raised against a polypeptide encoded by a wild-type or mutant LZTR1 or an epitope-containing fragment thereof. Although non-specific binding towards other antigens can occur, binding to the target protein occurs with a higher affinity and can be reliably discriminated from non-specific binding. In one embodiment, the method can comprise contacting a sample from the subject with an antibody specific for wild-type of mutant protein, and determining the presence of an immune complex. Optionally, the sample can be contacted to a support coated with antibody specific for a wild-type of mutant LZTR1 protein. In one embodiment, the sample can be contacted simultaneously, or in parallel, or sequentially, with various antibodies specific for different domains of a wild-type or mutant LZTR1 protein.

In some embodiments, the subject matter described herein provides for a diagnostic kit comprising products and reagents for detecting in a sample from a subject the presence of wild-type of mutant LZTR1 The kit can be useful for determining whether a sample from a subject exhibit increased or reduced expression of a LZTR1 protein. For example, the diagnostic kit according to the present invention comprises any primer, any pair of primers, any nucleic acid probe and/or any ligand, or any antibody directed specifically to a wild-type or mutant LZTR1 protein. The diagnostic kit according to the present invention can further comprise reagents and/or protocols for performing a hybridization, amplification, or antigen-antibody immune reaction. In one embodiment, the kit can comprise nucleic acid primers that specifically hybridize to and can prime a polymerase reaction from a wild-type or mutant LZTR1, or a combination thereof. In one embodiment, primers can be used to detect a wild-type or mutant LZTR1.

The diagnosis methods can be performed in vitro, ex vivo, or in vivo. These methods utilize a sample from the subject in order to assess the status of a LZTR1 protein. The sample can be any biological sample derived from a subject, which contains nucleic acids or polypeptides. Examples of such samples include, but are not limited to, fluids, tissues, cell samples, organs, and tissue biopsies. Non-limiting examples of samples include blood, liver, plasma, serum, saliva, urine, or seminal fluid. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. The nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes, primers, or ligands, in order to assess the presence of a wild-type or mutant LZTR1 protein. Contacting can be performed in any suitable device, such as a plate, tube, well, or glass. In some embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

In one embodiment, the biological sample comprises neuronal cells, serum, bone marrow, blood, peripheral blood, lymph nodes, cerebro-spinal fluid, urine, a saliva sample, a buccal swab, a serum sample, a sputum sample, a lacrimal secretion sample, a semen sample, a vaginal secretion sample, a fetal tissue sample, or a combination thereof.

### Administration of Pharmaceutical Composition

One or more inhibitors of the invention can be incorporated into pharmaceutical compositions suitable for administration, for example the inhibitor(s) and a pharmaceutically acceptable carrier. The inhibitor(s) of the invention can be administered to the subject once (e.g., as a single injection or deposition). Alternatively, the inhibitor(s) can be administered once or twice daily to a subject in need thereof for a period of from about two to about twenty-eight days, or from about seven to about ten days. The inhibitor(s) can also be administered once or twice daily to a subject for a period of 1, 2, 3, 4, 5, 6, 7, 8, 9, 10, 11, 12 times per year, or a combination thereof. The inhibitors can be administered concomitantly or consequentially. Furthermore, the inhibitor(s) of the invention can be co-administrated with another therapeutic.

The inhibitor(s) can be administered to a subject by any means suitable for delivering the inhibitor(s) to cells of the subject, such as cancer cells, e.g., glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes.

The pharmaceutical compositions of this invention can be formulated and administered to reduce the symptoms associated with a mutant LZRT1 protein-associated cancer, e.g., glioblastoma multiforme (GBM), Schwannoma, hepatocellular cancer, esophagogastric cancer, colorectal cancer, breast carcinoma, prostate cancer, lung adenocarcinoma, clonal hematopoiesis disorders, or tumor predisposition syndromes, by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

A therapeutically effective dose of inhibitor(s) described herein can depend upon a number of factors known to those or ordinary skill in the art. The dose(s) of the inhibitor(s) can vary, for example, depending upon the identity, size, and condition of the subject or sample being treated, further depending upon the route by which the composition is to be administered, if applicable, and the effect which the practitioner desires the inhibitor(s) to have upon the nucleic acid or polypeptide of the invention, such as the nucleic acid or polypeptide of a substrate of a mutant LZRT1. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the subject matter described herein can be formulated in conventional manner using one or more physiologically acceptable carriers or excipients. The therapeutic compositions of the invention can be formulated for a variety of routes of administration, including systemic and topical or localized administration. Techniques and formulations generally can be found in Remington's Pharmaceutical Sciences, Meade Publishing Co., Easton, Pa (20th Ed., 2000), the entire disclosure of which is herein incorporated by reference in its entirety. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogenfree. These pharmaceutical formulations include formulations for human and veterinary use.

According to the subject matter described herein, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition containing the inhibitor(s) described herein can be administered in conjunction with a pharmaceutically acceptable carrier, for any of the therapeutic effects discussed herein. Such pharmaceutical compositions can comprise, for example antibodies directed to a substrate of a LZRT1 protein, or a variant thereof, or antagonists of a substrate of a LZRT1 protein. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones. Sterile injectable solutions can be prepared by incorporating the inhibitor(s) (e.g., a small molecule or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration. This mode of administration can be feasible where the subcutaneous layer is sufficiently thin so that the factors present in the compositions can migrate or diffuse from the locus of administration. Thus, where intradermal administration is utilized, the bolus of composition administered is localized proximate to the subcutaneous layer. Other modes of application by multiple routes will be apparent to the skilled artisan.

A pharmaceutical composition of the invention is formulated to be compatible with its intended route of administration. Examples of routes of administration include parenteral, e.g., intravenous, intradermal, subcutaneous, oral (e.g., inhalation or ingestion), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the inhibitor (e.g., a polypeptide or antibody or small molecule) of the invention in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier and subsequently swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the effective amount of the administered inhibitor(s) is at least about 0.0001 μg/kg body weight, at least about 0.00025 μg/kg body weight, at least about 0.0005 μg/kg body weight, at least about 0.00075 μg/kg body weight, at least about 0.001 μg/kg body weight, at least about 0.0025 μg/kg body weight, at least about 0.005 μg/kg body weight, at least about 0.0075 μg/kg body weight, at least about 0.01 μg/kg body weight, at least about 0.025 μg/kg body weight, at least about 0.05 μg/kg body weight, at least about 0.075 μg/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 μg/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1,000 μg/kg body weight, at least about 2,000 μg/kg body weight, at least about 3,000 μg/kg body weight, at least about 4,000 μg/kg body weight, at least about 5,000 μg/kg body weight, at least about 6,000 μg/kg body weight, at least about 7,000 μg/kg body weight, at least about 8,000 μg/kg body weight, at least about 9,500 μg/kg body weight, or at least about 10,000 μg/kg body weight.

In some embodiments, osimertinib or a pharmaceutically acceptable salt thereof is administered at a dose of about 80 mg. In some embodiments, osimertinib or a pharmaceutically acceptable salt thereof is administered at a dose of about 40 mg, 50 mg, 60 mg, 70 mg, 80 mg, 90 mg, or 100 mg. In some embodiments, osimertinib or a pharmaceutically acceptable salt thereof is administered at a dose of 80 mg. In some embodiments, osimertinib or a pharmaceutically acceptable salt thereof is administered orally. In some embodiments, osimertinib or a pharmaceutically acceptable salt thereof is administered once daily. In some embodiments, osimertinib or a pharmaceutically acceptable salt thereof is administered with or without food.

In some embodiments, the pharmaceutical composition or pharmaceutical composition of kits described herein comprises osimertinib or a pharmaceutically acceptable salt thereof at a dose of about 80 mg. In some embodiments, the pharmaceutical composition or pharmaceutical composition of kits described herein comprises osimertinib or a pharmaceutically acceptable salt thereof at a dose of about 40 mg, 50 mg, 60 mg, 70 mg, 80 mg, 90 mg, or 100 mg. In some embodiments, the pharmaceutical composition or pharmaceutical composition of kits described herein comprises osimertinib or a pharmaceutically acceptable salt thereof at a dose of 80 mg. In some embodiments, the pharmaceutical composition or pharmaceutical composition of kits described herein comprises osimertinib or a pharmaceutically acceptable salt thereof for oral administration. In some embodiments, the pharmaceutical composition or pharmaceutical composition of kits described herein comprises osimertinib or a pharmaceutically acceptable salt thereof and a pharmaceutically acceptable excipient and/or carrier.

In some embodiments, afatinib or a pharmaceutically acceptable salt thereof is administered at a dose of about 40 mg. In some embodiments, afatinib or a pharmaceutically acceptable salt thereof is administered at a dose of about 10 mg, 20 mg, 30 mg, 40 mg, 50 mg, 60 mg, 70 mg, or 80 mg. In some embodiments, afatinib or a pharmaceutically acceptable salt thereof is administered at a dose of 40 mg. In some embodiments, afatinib or a pharmaceutically acceptable salt thereof is administered at a dose of 30 mg. In some embodiments, afatinib or a pharmaceutically acceptable salt thereof is administered at a dose of 30 mg in subjects with severe renal impairment. In some embodiments, afatinib or a pharmaceutically acceptable salt thereof is administered orally. In some embodiments, afatinib or a pharmaceutically acceptable salt thereof is administered once daily. In some embodiments, afatinib or a pharmaceutically acceptable salt thereof is administered with or without food.

In some embodiments, the pharmaceutical composition or pharmaceutical composition of kits described herein comprises afatinib or a pharmaceutically acceptable salt thereof at a dose of about 40 mg. In some embodiments, the pharmaceutical composition or pharmaceutical composition of kits described herein comprises afatinib or a pharmaceutically acceptable salt thereof at a dose of about 10 mg, 20 mg, 30 mg, 40 mg, 50 mg, 60 mg, 70 mg, or 80 mg. In some embodiments, the pharmaceutical composition or pharmaceutical composition of kits described herein comprises afatinib or a pharmaceutically acceptable salt thereof at a dose of 40 mg. In some embodiments, afatinib or a pharmaceutically acceptable salt thereof is administered at a dose of 30 mg. In some embodiments, the pharmaceutical composition or pharmaceutical composition of kits described herein comprises afatinib or a pharmaceutically acceptable salt thereof for oral administration. In some embodiments, the pharmaceutical composition or pharmaceutical composition of kits described herein comprises afatinib or a pharmaceutically acceptable salt thereof and a pharmaceutically acceptable excipient and/or carrier.

In some embodiments, bemcentinib or a pharmaceutically acceptable salt thereof is administered at a dose of about 200 mg. In some embodiments, bemcentinib or a pharmaceutically acceptable salt thereof is administered at a dose of about 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, bemcentinib or a pharmaceutically acceptable salt thereof is administered at a dose of 200 mg. In some embodiments, bemcentinib or a pharmaceutically acceptable salt thereof is administered orally. In some embodiments, bemcentinib or a pharmaceutically acceptable salt thereof is administered once daily. In some embodiments, bemcentinib or a pharmaceutically acceptable salt thereof is administered with or without food.

In some embodiments, the pharmaceutical composition or pharmaceutical composition of kits described herein comprises bemcentinib or a pharmaceutically acceptable salt thereof at a dose of about 200 mg. In some embodiments, the pharmaceutical composition or pharmaceutical composition of kits described herein comprises bemcentinib or a pharmaceutically acceptable salt thereof at a dose of about 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the pharmaceutical composition or pharmaceutical composition of kits described herein comprises bemcentinib or a pharmaceutically acceptable salt thereof at a dose of 200 mg. In some embodiments, the pharmaceutical composition or pharmaceutical composition of kits described herein comprises bemcentinib or a pharmaceutically acceptable salt thereof for oral administration. In some embodiments, the pharmaceutical composition or pharmaceutical composition of kits described herein comprises bemcentinib or a pharmaceutically acceptable salt thereof and a pharmaceutically acceptable excipient and/or carrier.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: LZTR1 Mutation Mediates Oncogenesis Through Stabilization of EGFR and AXL

LZTR1 is the substrate-specific adaptor of a CUL3-dependent ubiquitin ligase frequently mutated in sporadic and syndromic cancer. Biochemical and genetic studies were combined to identify LZTR1 substrates and interrogated their tumor-driving function in the context of LZTR1 loss-of-function mutations. Unbiased screens converged on EGFR and AXL receptor tyrosine kinases as LZTR1 interactors targeted for ubiquitin-dependent degradation in the lysosome. Pathogenic cancer-associated mutations of LZTR1 failed to promote EGFR and AXL degradation, resulting in dysregulated growth factor signaling. Conditional inactivation of Lztr1 and Cdkn2a in the mouse nervous system caused tumors in the peripheral nervous system including schwannoma-like tumors, thus recapitulating aspects of Schwannomatosis, the prototype tumor predisposition syndrome sustained by LZTR1 germline mutations. Lztr1 and Cdkn2a deleted tumors aberrantly accumulated EGFR and AXL and exhibited specific vulnerability to EGFR and AXL co-inhibition. These findings explain tumorigenesis by LZTR1 inactivation and offer therapeutic opportunities to patients with LZTR1 mutant cancer.

### Introduction

Leucine zipper-like transcription regulator 1 (LZTR1) is a Kelch-BTB (broad complex, tramtrack, and bric à brac)-BACK (BTB and C-terminal Kelch) domain-containing protein that functions as substrate adaptor of a Cullin 3 (CUL3)-RING E3 ubiquitin Ligase (CRL) complex CRL3^{LZTR1} (1,2). To date a full characterization of the function of the LZTR1 protein is still lacking but the interest in LZTR1 function continues to increase due to the growing number of cancer types that are reported to harbor genetic alterations of the LZTR1 gene. After our initial discovery of LZTR1 mutations and deletions in glioblastoma multiforme (GBM) (1), genetic alterations of LZTR1 have been identified in numerous cancers including hepatocellular, esophagogastric, colorectal and breast carcinoma, lung adenocarcinoma, clonal hematopoiesis disorders and a variety of human tumor predisposition syndromes (1,3-16). For example, germline mono-allelic mutations of LZTR1 were first reported in patients with Schwannomatosis, a genetic disorder characterized by highly invalidating tumors of the peripheral nervous system (PNS) (4,17). Mutations of LZTR1 involve both Kelch and BTB-BACK domain, substrate and CUL3 interacting domain, respectively, albeit without clustering in hot spots. In vitro experiments and data from human genetic studies indicated that LZTR1 mutations are loss-of-function events and pointed to a potential tumor suppressor function for this gene (1,3-5). However, the role of LZTR1 as tumor suppressor gene in the whole organism remained to be charted.

Similarly, the identity of the protein substrates targeted by LZTR1-mediated ubiquitylation and the oncogenic functions implemented by deregulation of specific LZTR1-substrate(s) in LZTR1-mutant disorders remained puzzling. While initial reports suggested that RAS proteins interact with LZTR1 and undergo direct ubiquitylation by CRL3^{LZTR1} complexes (18,19), later studies failed to confirm these interactions (20). Instead, the RAS family member RIT1 was identified as substrate of LZTR1 through RIT1-specific protein interaction assay and dysregulation of RIT1 was suggested to contribute to the activation of MAP kinase signaling in Noonan syndrome caused by pathogenic mutations of LZTR1 (20). However, this study left open the question of the broad cellular scope of LZTR1 substrates and especially their role in sporadic tumors and cancer predisposition syndromes such as Schwannomatosis sustained by LZTR1 inactivation.

Here, multiple unbiased proteomics screens were used to capture substrates of LZTR1-mediated ubiquitylation/degradation and uncovered EGFR and AXL, two oncogenic receptor tyrosine kinases (RTK s), as novel protein substrates of LZTR1 activity. Interaction with and ubiquitylation by LZTR1 of both RTKs was consequent to ligand-induced activation, leading to lysosomal mediated degradation and signaling downregulation. The LZTR1 tumor suppressor function was modeled in a mouse model sustained by genetic inactivation of Lztr1 and Cdkn2a in the nervous system. Compound mice developed tumors resembling neurofibroma, malignant PNS tumors and schwannomas, all characterized by the elevated expression of EGFR and AXL. Finally, LZTR1 germline and somatic mutations identified in cancer patients fail to balance EGFR and AXL cellular levels. Consequently, LZTR1 inactivation induces vulnerability to the concurrent inhibition of the two RTKs, thus providing new treatment opportunities to patients affected by cancer harboring mutation of LZTR1.

### Results

### Unbiased Identification of LZTR1 Substrates

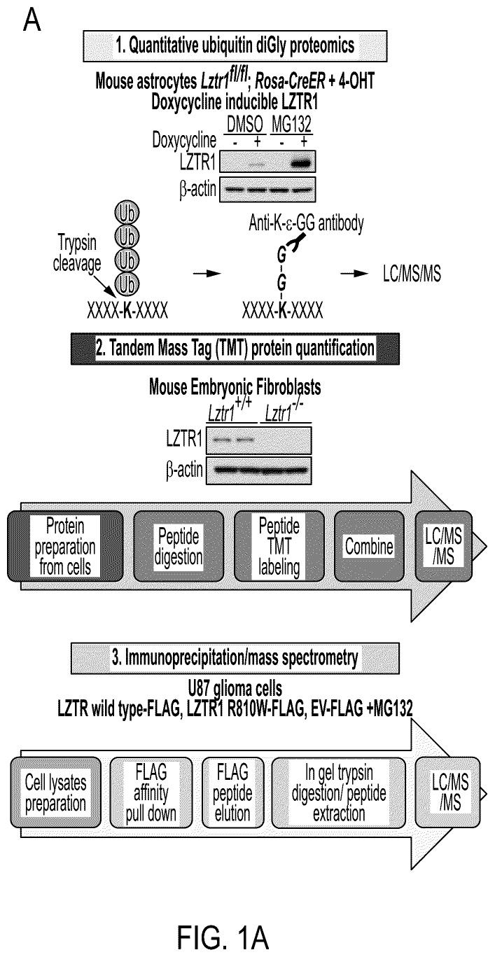

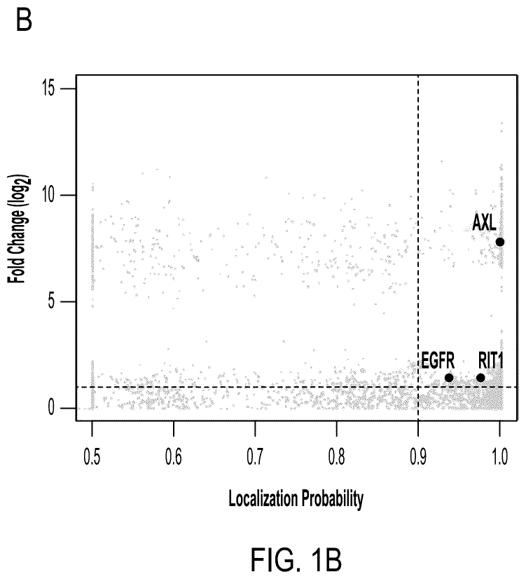

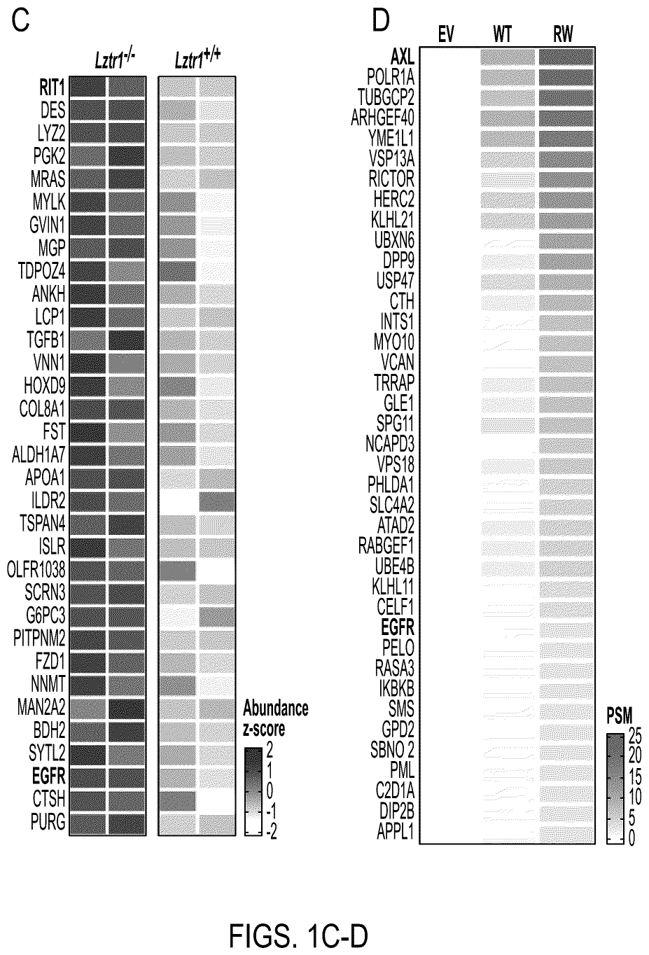

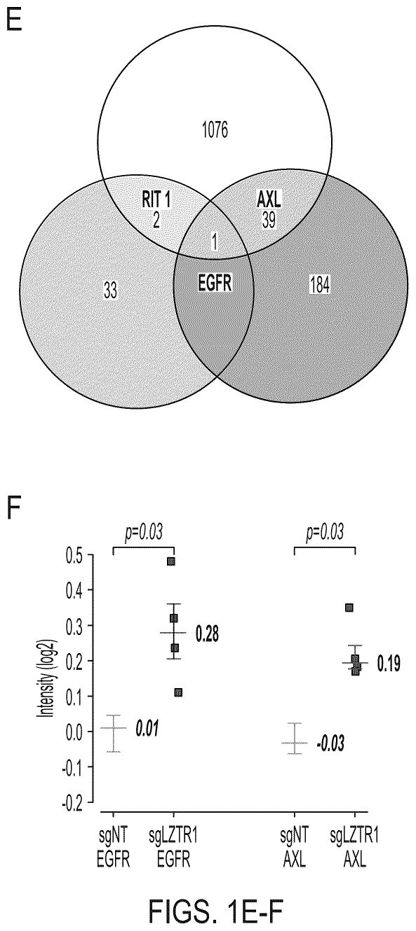

To identify the protein substrates targeted by LZTR1-dependent ubiquitin-mediated degradation, three orthogonal and unbiased screens were performed (FIG. 1A). First, to uncover ubiquitylation targets of LZTR1, quantitative ubiquitin di-glycine (diGly) proteomics, a peptide antibody-based affinity approach that enriches for and identifies endogenously ubiquitylated proteins, was performed using Lztr1^{fl/fl};Rosa-CreER mouse astrocytes. In this cellular system, the endogenous Lztr1 gene was deleted by 4-hydroxytamoxifen (4-OHT)-induced recombination and LZTR1 re-expressed by the tetracycline-inducible system (FIG. 1A). 1,076 proteins were found to have higher ubiquitylation in LZTR1-reconstituted cells compared with Lztr1-deleted astrocytes (FIG. 1B; List 1 (see below)). Second, Tandem M as Tag (TMT)-based quantitative proteomics was used to identify proteins elevated by genetic deletion of the Lztr1 gene in mouse embryonic fibroblasts (MEFs) (FIG. 1A). A set of 33 proteins were detected that accumulated at higher levels in constitutive Lztr1 knockout MEFs (FIG. 1C; List 2 (see below)). Third, the LZTR1 cellular interactome was probed by immunoprecipitation/mass spectrometry of U87 human glioma cells stably expressing LZTR1 wildtype or the LZTR1-R810W mutant that was initially reported as somatic mutation in human glioblastoma (GBM) (FIG. 1A). The LZTR1-R810W protein harbors a loss-of-function mutation in the BTB-BACK domain that impairs CUL3 recognition, thus stabilizing the protein without affecting the substrate-interacting Kelch domain (1). The analysis uncovered 184 proteins specifically interacting with LZTR1 wild type and LZTR1-R810W mutant with spectral counts >2-fold in LZTR1-R810W compared with LZTR1 wild type (FIG. 1D; List 3 (see below)), a criterion that was applied to identify candidate substrates of LZTR1, which more stably interact with the ineffective mutant. The receptor tyrosine kinase (RTK) epidermal growth factor receptor (EGFR) was the only hit common to all three screens (FIG. 1B-E). Another oncogenic RTK, AXL (21) also scored as positive hit in two of the three screens (FIG. 1B, D and E). RIT1, a RAS-related small GTPase recently reported as substrate of LZTR1 (20), also emerged as positive hit from two of the three screens (FIG. 1B, C and E). Using independent TMT-based quantitative proteomics experiments in Hela cells, it was confirmed that CRISPR-Cas9-mediated knockout of LZTR1 consistently increased EGFR and AXL proteins compared with control cells (FIG. 1F).

### LZTR1 Induces Ubiquitin-Mediated Degradation of EGFR and AXL

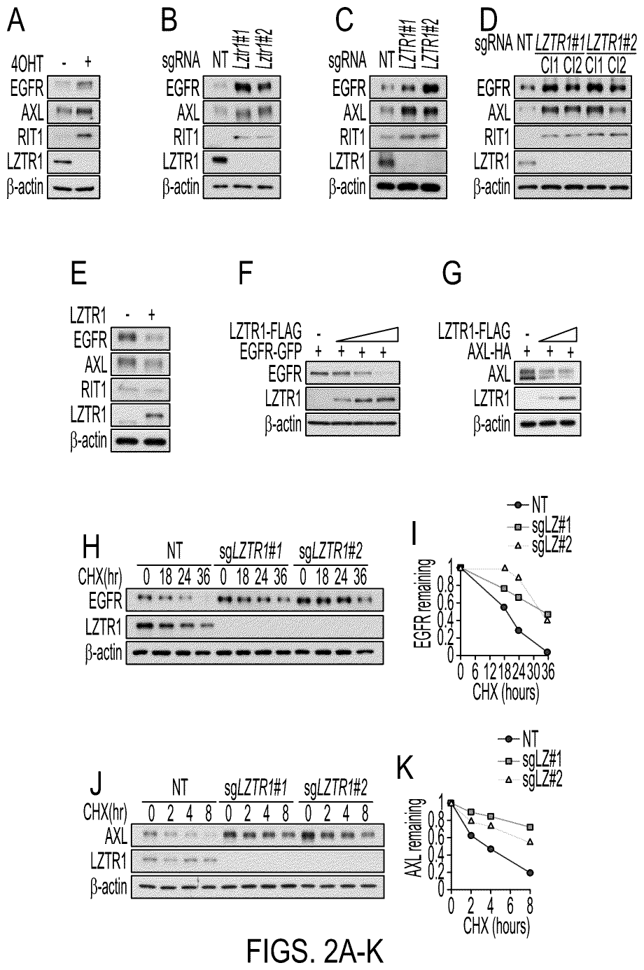

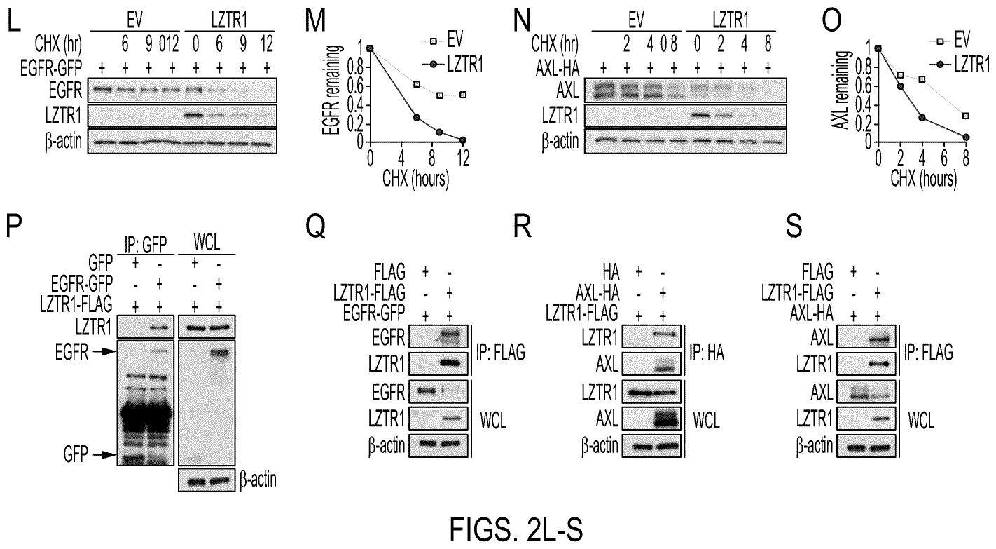

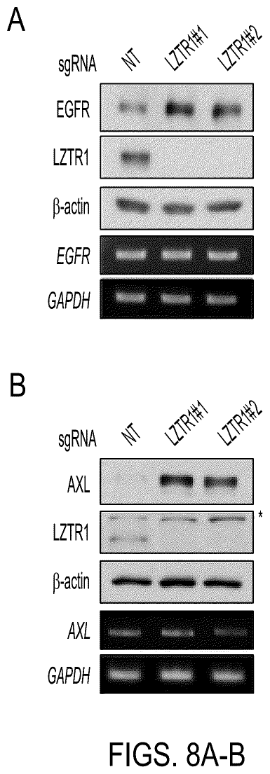

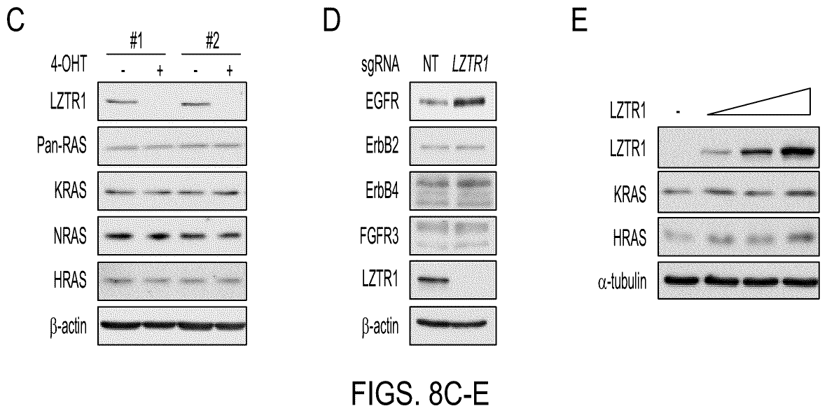

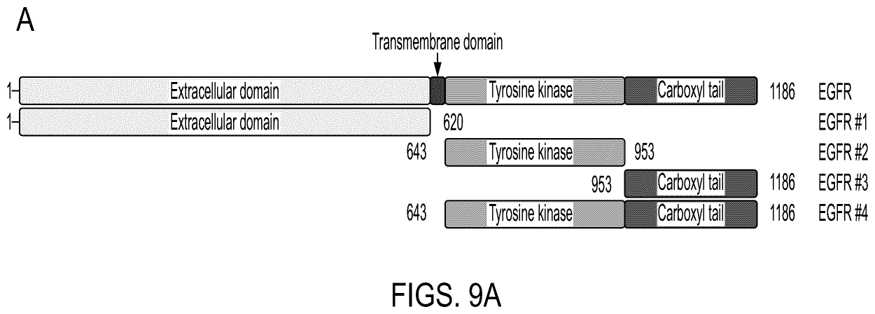

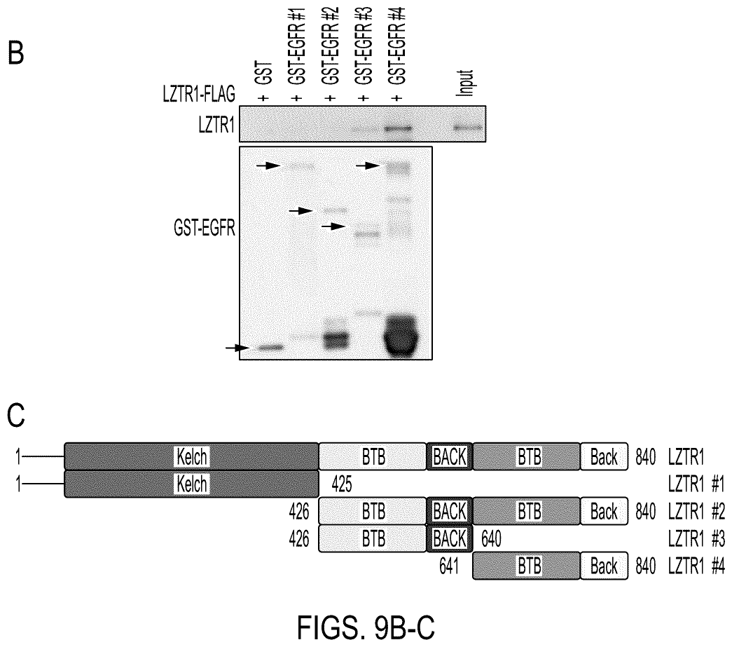

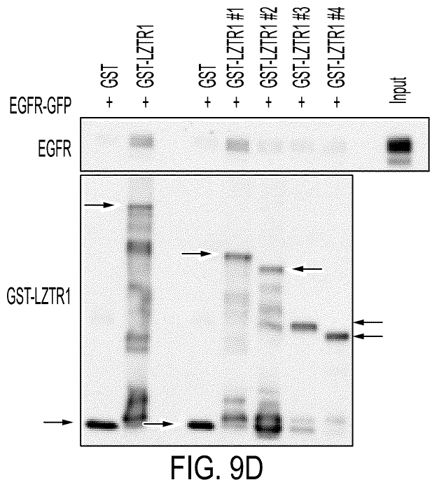

Given the large spectrum of cancer types harboring genetic alterations of LZTR1, it was sought to define the activity of LZTR1 in cells originating from different tissues and the relationship with the other described LZTR1 substrates. Genetic deletion of the Lztr1 gene in mouse astrocytes and CRISPR-Cas9 mediated knockout of LZTR1 in immortalized Schwann cells SW10, U87 malignant glioma, and Hela cells consistently induced accumulation of EGFR and AXL in the absence of change of mRNA (FIG. 2A-D; FIG. 8A and B). Accumulation of RIT1, was also readily detected in LZTR1-deleted cells (FIG. 2A-D). However, deletion of LZTR1 did not change the steady state levels of any of the RAS isoform, K-RAS, H-RAS and N-RAS (FIG. 8C). Similarly unchanged were the levels of ERBB2, ERBB4, and FGFR3, thus highlighting the specificity of LZTR1 activity towards the RTKs EGFR and AXL (FIG. 8D). The consequences of ectopically increasing LZTR1 expression on endogenous and exogenous EGFR and AXL was then tested in several cell types. Expression of LZTR1 in SW10 cells caused a noticeable decrease of endogenous EGFR and AXL proteins but only minimal change of RIT1 (FIG. 2E) and led to destabilization of exogenously expressed EGFR and AXL in a dose dependent manner (FIG. 2F and G). RAS isoforms were not affected by LZTR1 expression (FIG. 8E). The effect of LZTR1 on EGFR and AXL protein stability was confirmed by the markedly prolonged half-life under cycloheximide (CHX) treatment in cells with knockout of LZTR1 (FIG. 2H-K) and accelerated protein turn-over by increased LZTR1 (FIG. 2L-O). Finally, immunoprecipitation from lysates of cells expressing LZTR1-FLAG and EGFR-GFP or AXL-HA demonstrated that LZTR1 physically interacts with EGFR (FIG. 2P and Q) and AXL (FIG. 2R and S). GST pull-down assays with different GST-EGFR protein domains and LZTR1-FLAG from HEK-293T cell lysates revealed that the intracellular domain of EGFR interacts with LZTR1 (FIG. 9A and B). Reciprocal experiments with purified GST-LZTR1 deletion polypeptides identified the Kelch domain of LZTR1 as the EGFR-binding region (FIG. 9C and D).

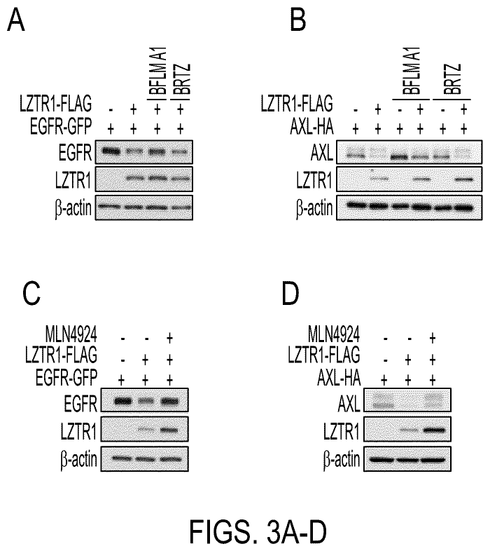

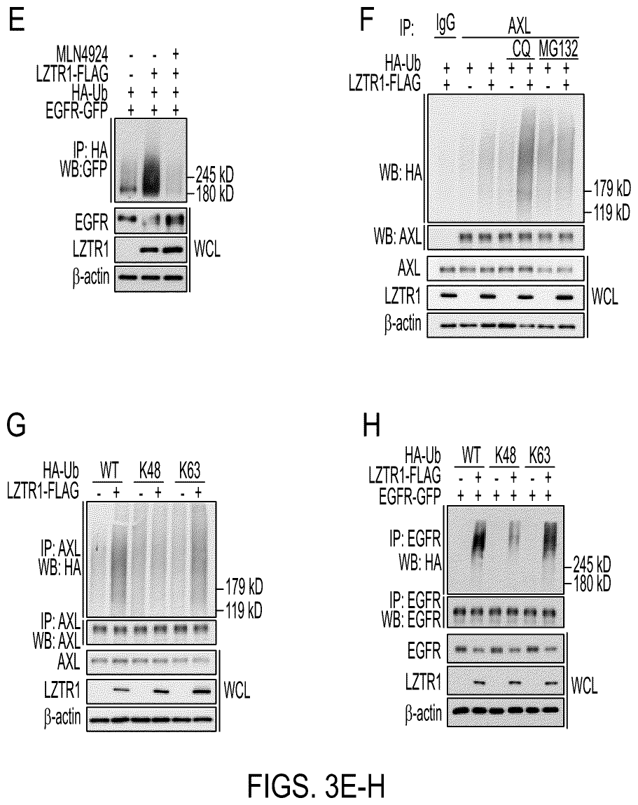

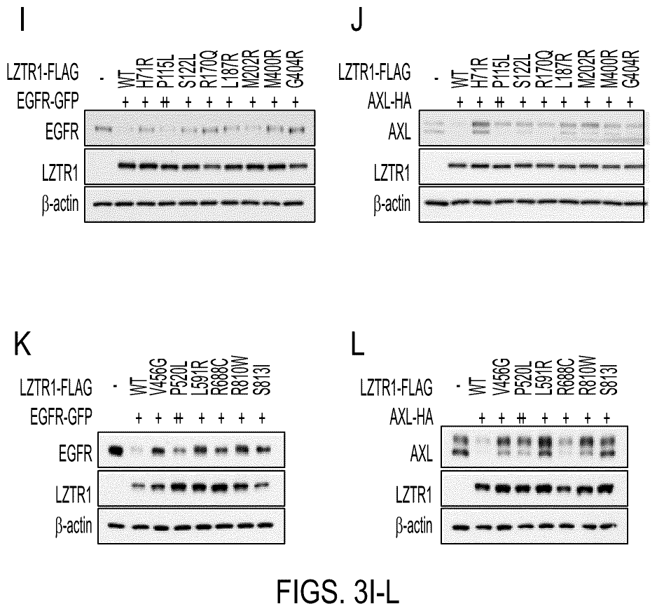

Following ligand-mediated activation and endocytosis, RTK s are targeted to the lysosome for degradation (22-24). LZTR1-mediated destabilization of EGFR and AXL was relieved by the lysosomal inhibitor bafilomycin A1 (BFLMA1) but not the proteasome inhibitor bortezomib (BRTZ) (FIG. 3A and B). In contrast, RIT1 reduction by LZTR1 was rescued by bortezomib but not bafilomycin A1, a finding consistent with the engagement of different proteolytic machinery for these substrates of LZTR1 (FIG. 10A). Confirming published findings (20), RIT1 physically interacted with LZTR1 in the absence of association with EGFR and AXL, thus suggesting independent regulation of substrates by LZTR1 (FIG. 10B). Consistent with the CULLIN-mediated mechanism of degradation by the CRL3LZTR1 complex, the NEDD8-activating enzyme (NAE) inhibitor MLN4924, which blocks neddylation of CULLIN and inactivates CRLs (25), rescued LZTR1-induced destruction of EGFR, AXL and RIT1 (FIG. 3C and D; FIG. 10C). Next, the ubiquitylation status of EGFR and AXL in the presence of LZTR1 was examined. Expression of LZTR1 increased the formation of high molecular weight polyubiquitylated EGFR and this effect was abolished when cells were treated with MLN4924 (FIG. 3E). LZTR1 also increased the polyubiquitylated species of AXL, which accumulated at much higher levels when cells were treated with the lysosome inhibitor chloroquine (CQ), whereas the proteasome inhibitor MG132 was ineffective (FIG. 3F). When co-expressed with ubiquitin wild type or ubiquitin variants in which all lysine residues had been changed to arginine except for lysine-48 (K48) or lysine-63 (K63), LZTR1 induced EGFR and AXL ubiquitylation by ubiquitin wild type and predominantly K63 but minimally K48, indicating that LZTR1 promotes preferentially K63-mediated ubiquitylation linkage, which is the key signal for efficient targeting of RTKs to the lysosomal degradation pathway (FIG. 3G and H) (26).

Missense point mutations of LZTR1 that have been reported in sporadic tumors and the germline of patients with Schwannomatosis target the Kelch domain, which is required for substrate recognition (1,4,8,27,28) but also the BTB-BACK domains, which interacts with CUL3 (FIG. 10D). To interrogate the cancer-specific significance of the inactivation of EGFR and AXL by LZTR1 in patients, we analyzed a panel of mutations of LZTR1 and found that most mutants compromised the degradation capacity of LZTR1 towards EGFR and AXL (FIG. 3I and J, for mutants in the Kelch domain; FIG. 3K and L for mutants in the BTB-BACK region). Consistent with impaired degradation of EGFR, most Kelch domain mutated proteins failed to interact with EGFR in co-immunoprecipitation assays (FIG. 10E). RIT1 destabilization was also impaired by all LZTR1 mutants tested except the R170Q mutation, which exhibited an activity comparable to LZTR1 wild type although it was ineffective towards AXL and EGFR (FIG. 10F). Therefore, LZTR1 mutations that are causally implicated in cancer and Schwannomatosis generally compromise the ability of this ubiquitin ligase to regulate EGFR and AXL protein balance.

LZTR1 Recognizes Activated EGFR and AXL and Targets them to the Lysosome

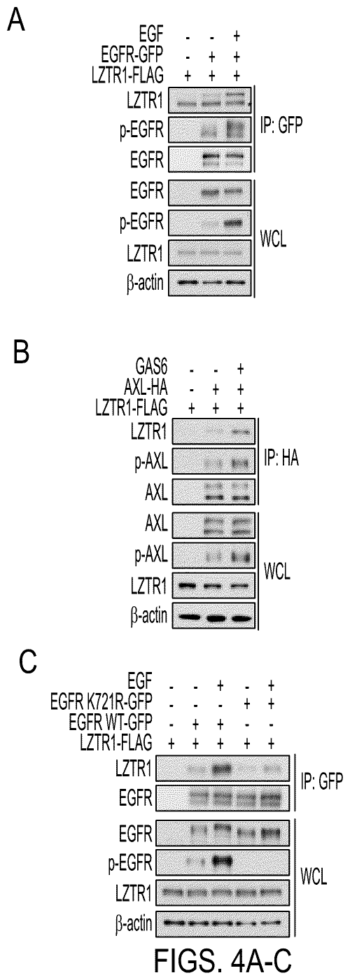

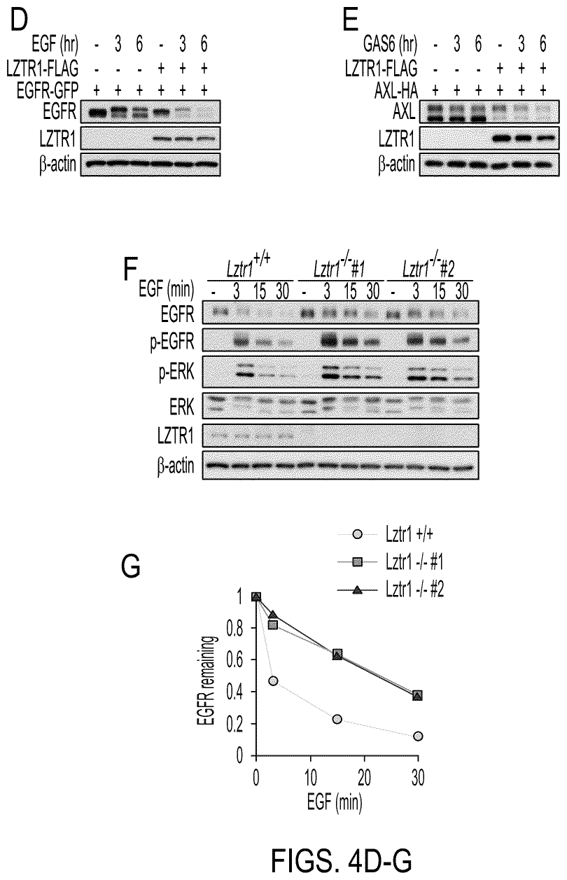

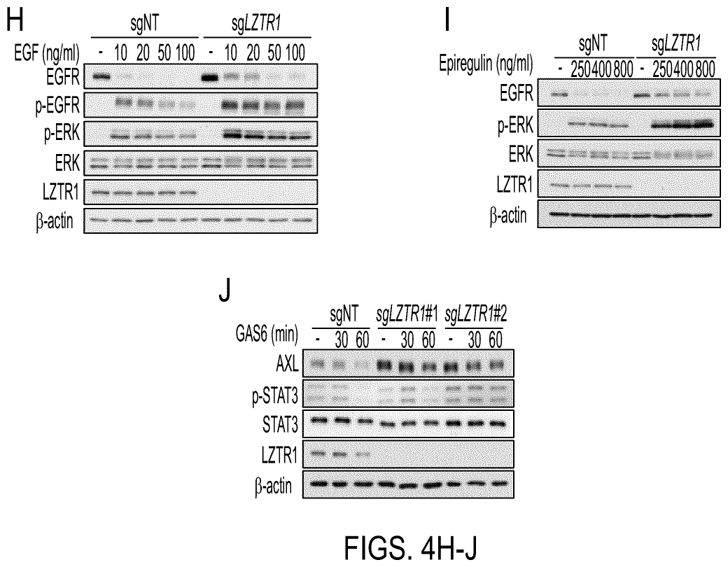

RTK signaling begins with the ligand-receptor interaction resulting in receptor dimerization, autophosphorylation of the C-terminal tail and recruitment of transducers of signaling. As negative feedback regulation, ligand binding to RTK s also triggers internalization and degradation of the activated receptor with consequent signal termination (29-31). It was found that EGF and GAS6 stimulation promoted the interaction of LZTR1 with the active, phosphorylated form of EGFR and AXL, respectively when tested by co-immunoprecipitation (FIG. 4A and B). EGF-induced stimulation of LZTR1-EGFR complex formation was impaired for the kinase-defective mutant EGFR-K721R, further supporting the conclusion that EGFR phosphorylation and activation promotes binding to LZTR1 (FIG. 4C). To directly determine whether the interaction with LZTR1 controls EGFR and AXL degradation initiated by EGF and GAS6, respectively, cells expressing EGFR or AXL were treated with EGF or GAS6 for different times in the presence or absence of LZTR1. LZTR1 markedly increased the degradation of EGFR and AXL at the times analyzed (FIG. 4D and E). LZTR1 also enhanced the EGF-induced ubiquitylation of EGFR (FIG. 10G). Conversely, constitutive Lztr1 deletion in MEFs impaired EGF-induced quenching of EGFR protein and signaling, maintaining active phospho-EGFR and preventing the loss of phospho-ERK (FIG. 4F and G). Similarly, CRISPR-Cas9-mediated knockout of LZTR1 in Hela cells stabilized EGFR and phospho-EGFR and resulted in persistent activation of MAPK when the signaling mechanisms were interrogated in the context of increasing doses of EGF (FIG. 4H). Knockout of LZTR1 caused comparable stabilization of EGFR and increased signaling when the receptor was activated by epiregulin, an EGFR low affinity ligand (32), indicating that the negative regulation of LZTR1 upon EGFR is independent of the particular ligand activating the receptor (FIG. 4I). Similarly, knockout of LZTR1 attenuated Gas6-mediated AXL degradation and prolonged STAT3 signaling (FIG. 4)).

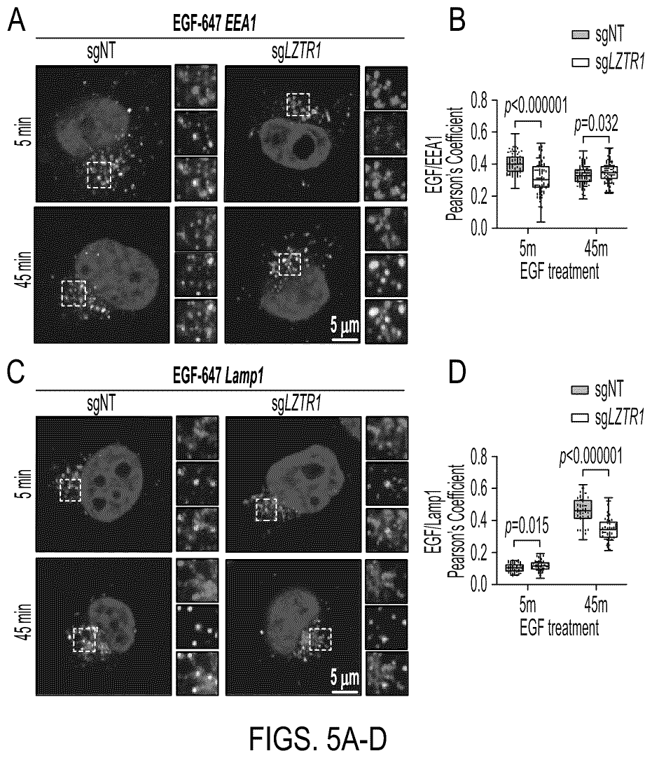

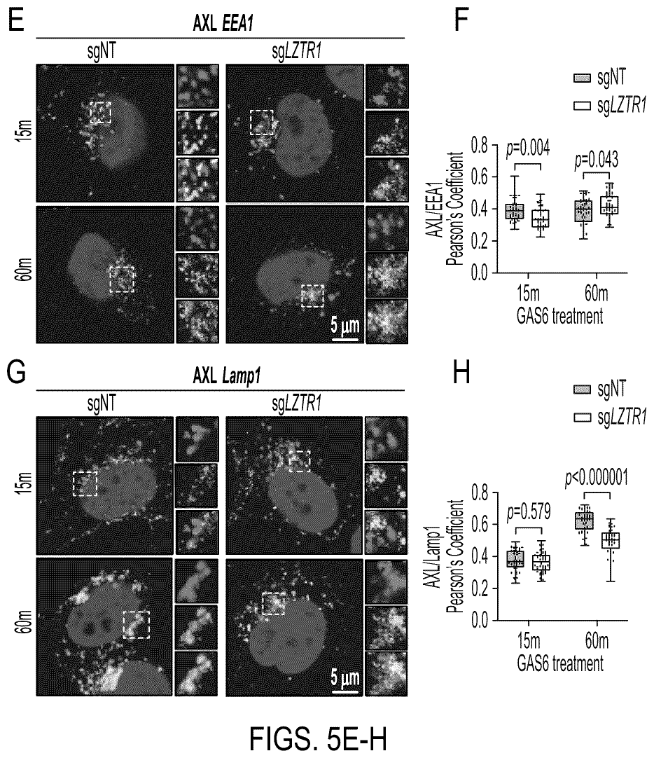

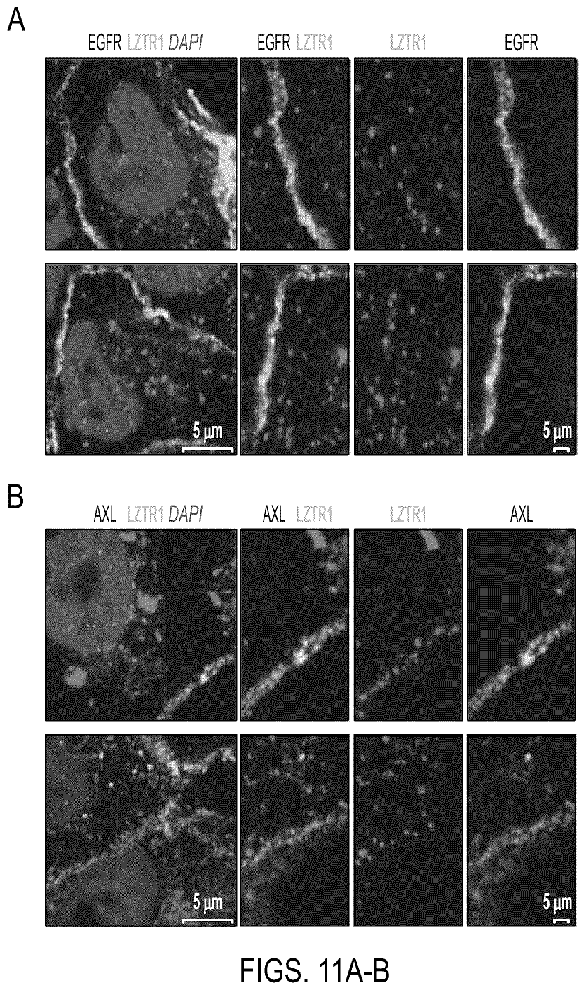

Receptor ubiquitylation is a sorting signal that targets activated RTK's at the cell surface to the lysosome by trapping them within clathrin-coated pits and multivesicular endosomes (33). Ubiquitylation of RTK s is not necessary for internalization but is required for lysosome targeting and degradation (34,35). To determine the role of LZTR1 for EGFR and AXL endocytosis and trafficking to the lysosome, the subcellular localization of LZTR1 in relationship with EGFR and AXL upon ligand stimulation was first examined. When expressed as EGFR-GFP fusion, the GFP signal co-localized with LZTR1-FLAG at the cell periphery 5 min after addition of EGF at 4° C. (FIG. 11A). Endogenous AXL and LZTR1 co-localized at the cellular membrane after addition of GAS6 to the culture (FIG. 11B). Next, the localization of ligand-occupied EGFR and AXL in LZTR1 wild type and knockout cells was analyzed. For EGFR-EGF assay EGF conjugated with Alexa-647 was used to track ligand-receptor interaction. In control cells analyzed five minutes after cell stimulation with EGF-Alexa-647, the EGF-EGFR ligand-receptor complex co-localized with the early endosome marker EEA1 and this co-localization decreased 45 minutes after EGF stimulation (FIG. 5A and B, left most bars for each condition). As expected, co-localization of EGF-Alexa-647 with the lysosomal marker Lamp1 was minimal 5 minutes after EGF stimulation and increased after 45 minutes (FIG. 5C and D, left most bars for each condition). In cells harboring knockout of LZTR1, a reduction of the EGF-EEA1 early endosome co-localization was initially detected 5 minutes after EGF addition but the early endosome compartmentalization of the EGF-EGFR complex remained stable after 45 minutes from EGF stimulation, thus persisting at levels slightly higher than those of control cells (FIG. 5A and B, right most bars for each condition). Loss of LZTR1 impaired lysosomal targeting of EGFR, as shown by the markedly reduced pool of EGFR co-localizing with the lysosomal marker LAMP1 45 minutes after EGF stimulation compared to control cells (FIG. 5C and D, right most bars for each condition). For the AXL-GAS6 assay, AXL trafficking after GAS6 addition was tracked by immunofluorescence of the endogenous AXL protein. As for EGFR, co-localization of AXL with EEA1 was reduced in LZTR1 knockout cells 15 minutes after stimulation with GAS6 compared with wild type cells (FIG. 5E and F, right most bars for each condition). A comparable statistically significant reduction in co-localization of AXL with LAMP1 was obtained in LZTR1 knockout cells relative to wild type when the analysis was performed 60 min after stimulation with GAS6 (FIG. 5G and H, right most bars for each condition). These results indicate that in the absence of LZTR1, EGFR and AXL are not efficiently sorted to the lysosome but remain in the early endosomal compartment and are likely recycled to the plasma membrane with defective termination of signaling.

### LZTR1 is a Tumor Suppressor in the Peripheral Nervous System

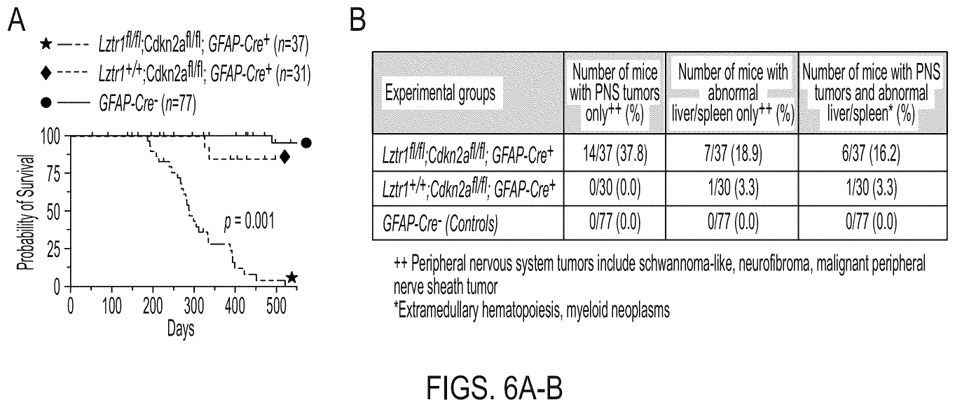

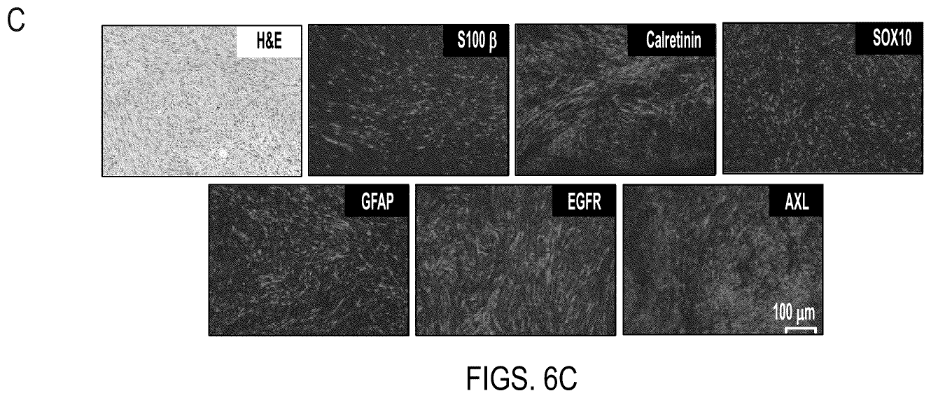

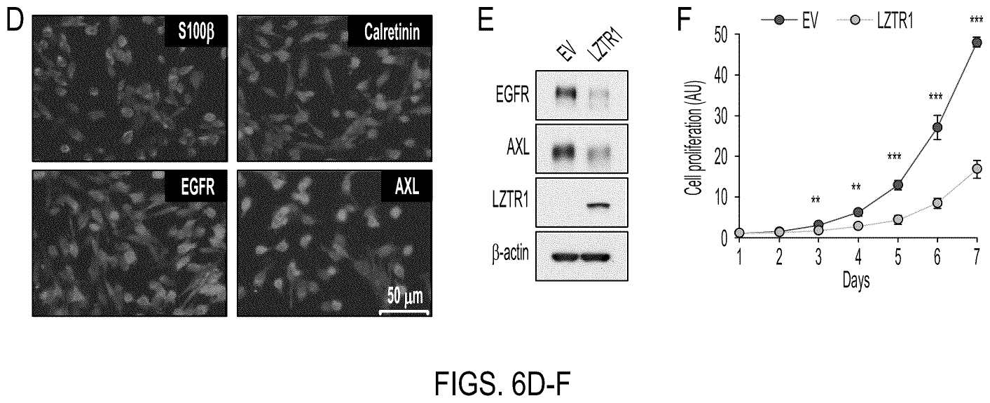

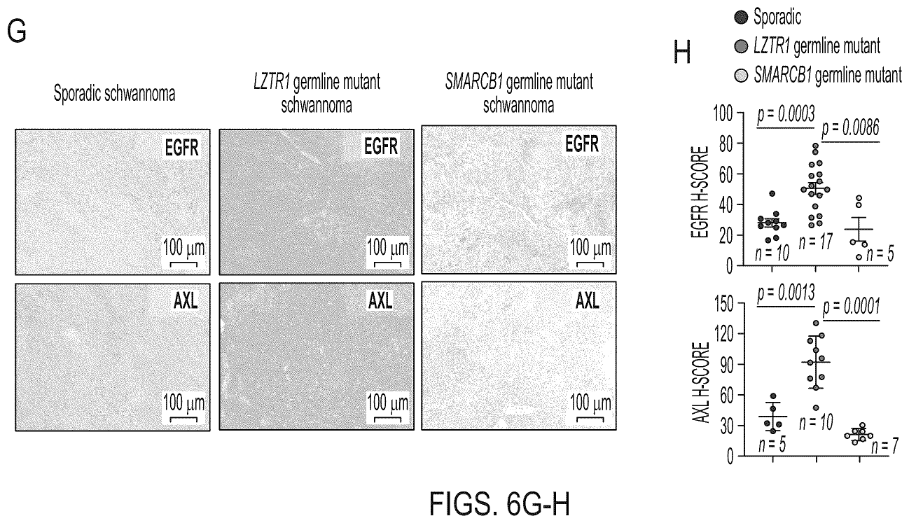

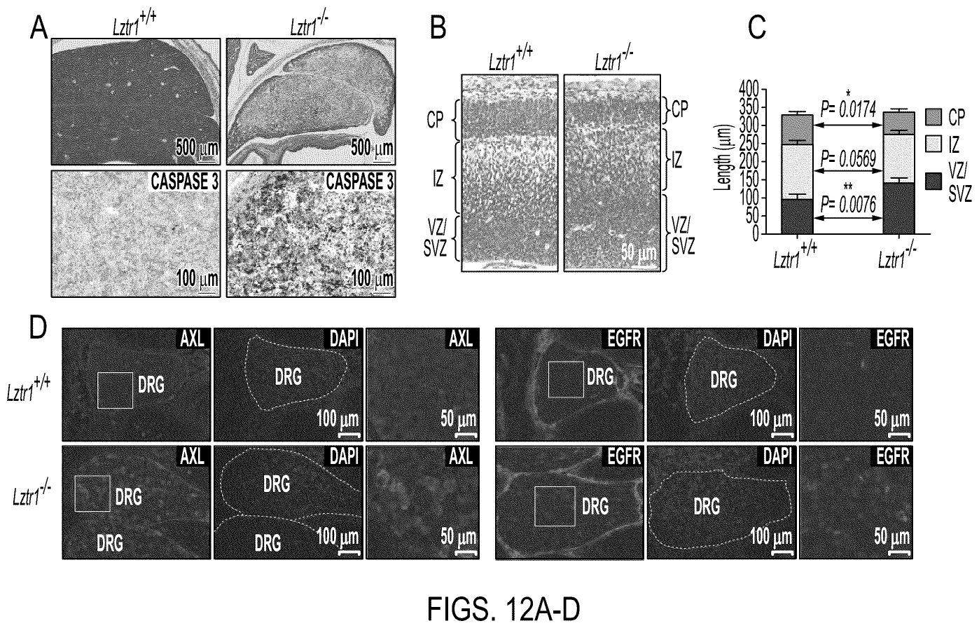

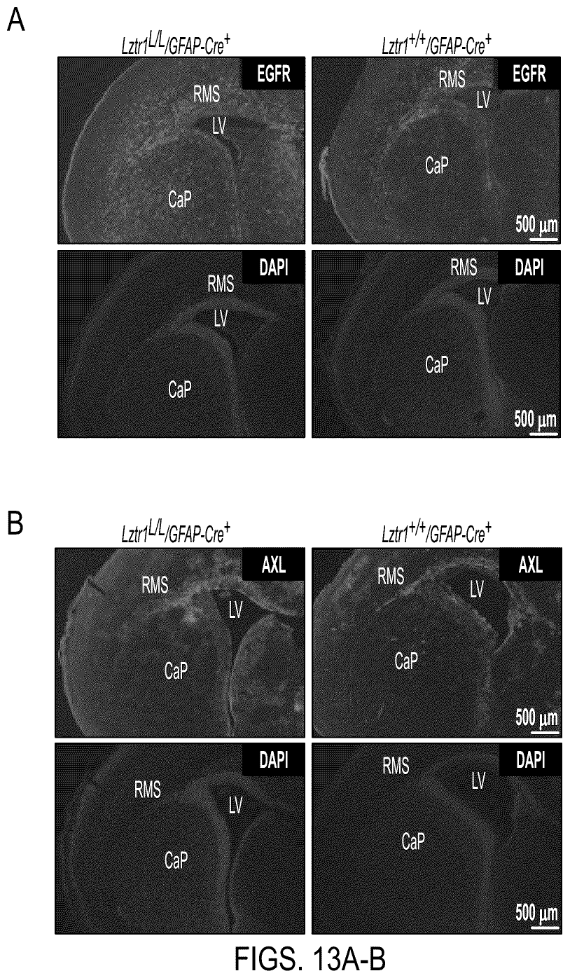

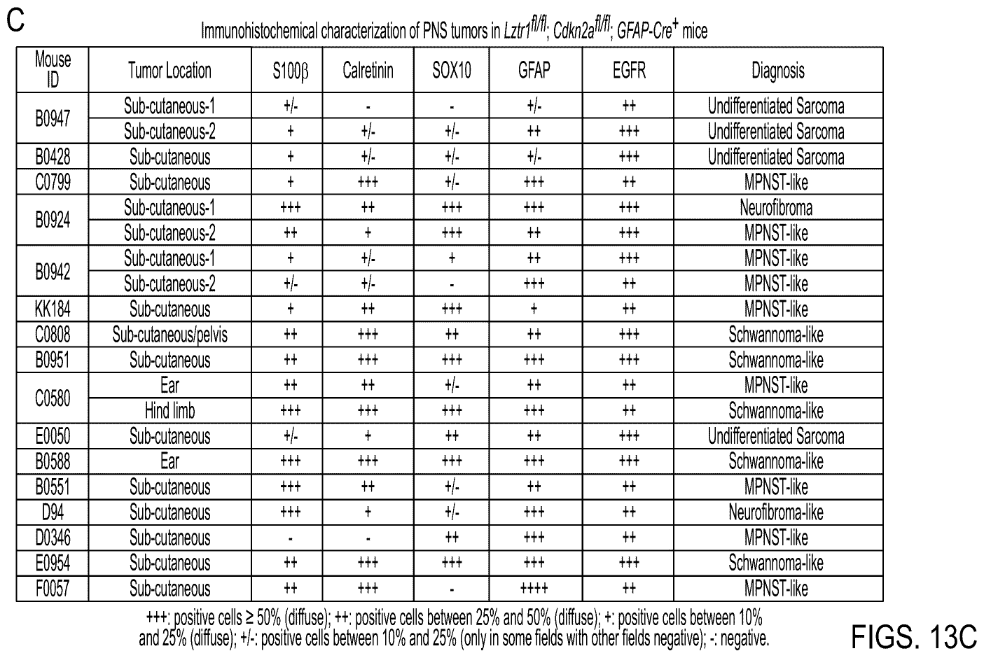

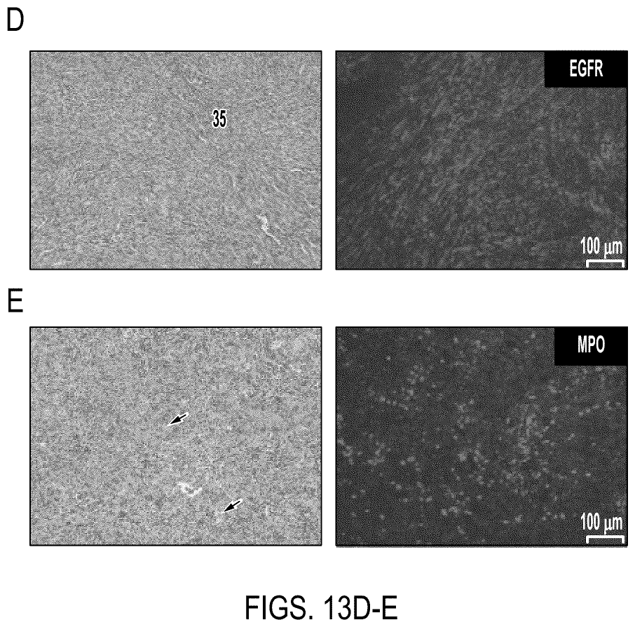

In humans, the LZTR1 gene is targeted by loss of function somatic mutations in numerous cancers and the germ line of patients with Schwannomatosis (1,3-16). To ask whether genetic deletion of Lztr1 in the mouse delivers a model of tumorigenesis and whether loss of LZTR1 activity in the whole organism is associated with accumulation of EGFR and AXL, constitutive and conditional Lztr1 knockout mice were generated (36). Constitutive Lztr1−/− mice died at mid-gestation with severe and progressive apoptotic cell death of liver cells (FIG. 12A). Embryos also exhibited abnormal expansion of the ventricular/subventricular zone (VZ/SVZ) of the telencephalon and a reduced cortical plate (CP), suggestive of a relative block of neurogenesis and neuronal differentiation (FIG. 12B and C). At this embryonic stage, dorsal root ganglia (DRG) can be clearly identified (37,38). Although expression of AXL and EGFR is tenuous in these structures, Lztr1-deleted DRG had higher levels of AXL and EGFR than wild type DRG (FIG. 12D). To explore the consequences of targeted deletion of Lztr1 in the nervous system, Lztr1^{fl/fl} mice were crossed with the GFAP-Cre deleter strain. GFAP, a marker of neural stem cells and astrocytes in the CNS, is expressed by immature Schwann cells and neural crest-derived skin precursors that have been proposed as cell of origin of peripheral nerve sheet tumors (PNSTs) in humans and mice (39-41). Lztr1^{fl/fl};GFAP-Cre mice were viable, exhibited minor neurological symptoms such as abnormality of the hind limb reflex and were tumor-free. EGFR and AXL protein was analyzed by immunofluorescence and found that they accumulated in Lztr1^{fl/fl};GFAP-Cre mouse brain at levels higher than the wild type littermates (FIG. 13A and B). While EGFR expression appeared widespread, expression of AXL accumulated in the area surrounding the lateral ventricles and the rostral migratory stream (FIG. 13B). The absence of tumor formation in the nervous system of the Lztr1 conditional knockout mouse was in line with the absence of tumor phenotypes in mice harboring conditional deletion of individual tumor suppressor genes (42-44). To ask whether LZTR1 loss cooperates with inactivation of other tumor suppressor genes for tumor development in the nervous system, Lztr1^{fl/fl};Cdkn2A^{fl/fl};GFAP-Cre compound mice were generated. Loss of function germline mutations of CDKN2A occur in the germline of patients predisposed to tumors in the PNS. Somatic deletions of CDKN2A are frequently associated with progression to malignancy of PNS tumors and are the most frequent genetic alterations in malignant peripheral nervous system tumors (MPNSTs) (45-50). Lztr1^{fl/fl};Cdkn2A^{fl/fl};GFAP-Cre mice exhibited significantly reduced survival compared with Lztr1+/+;Cdkn2A^{fl/fl};GFAP-Cre and control mice (FIG. 6A). The primary reason for lethality was tumor development in the PNS and hematopoietic lineage (FIG. 6B). Conversely, tumors in the brain of Lztr1^{fl/fl};Cdkn2A^{fl/fl};GFAP-Cre mice were not observed. Approximately 50% of Lztr1^{fl/fl};Cdkn2A^{fl/fl};GFAP-Cre mice developed PNS tumors in the skin, soft tissues and dorsal root ganglia that showed positivity for neural cell markers (FIG. 6C; FIG. 13C). Approximately 30% of the PNS tumors exhibited histological and immunophenotypic features compatible with schwannomas including a tendency to form pseudo-palisading structures, positivity for the Schwann cell markers S100β and SOX10, and diffuse positivity for calretinin, a neural protein recently included in the panel of markers differentiating schwannoma from neurofibroma in humans (FIG. 6C; FIG. 13C) (51,52). The morphology and immunophenotyping of tumors not classified as schwannoma-like in Lztr1^{fl/fl};Cdkn2A^{fl/fl};GFAP-Cre mice confirmed their origin from cells from the PNS and broadly indicated transition towards more malignant and anaplastic lesions, similar to MPNSTs. EGFR and AXL accumulated at high levels in schwannoma-like and MPNST-like tumors (FIG. 6C; FIG. 13D). Cells isolated from schwannoma-like tumors that developed in Lztr1^{fl/fl};Cdkn2A^{fl/fl};GFAP-Cre+ mice (E0954) were expanded ex vivo and retained high expression of the Schwann cell markers S100β and calretinin (FIG. 6D). They also exhibited elevated EGFR and AXL, which were downregulated following lentivirus-mediated reconstitution of LZTR1 (FIG. 6E), leading to reduced cell proliferation (FIG. 6F). In addition to tumors of the PNS, 36% of the Lztr1^{fl/fl};Cdkn2A^{fl/fl};GFAP-Cre mice developed splenomegaly and enlarged liver associated with myeloproliferative disorders spanning from extramedullary hematopoiesis to overt myelogenous leukemia (FIG. 13E). The spectrum of myeloid tumors observed in Lztr1^{fl/fl};Cdkn2A^{fl/fl};GFAP-Cre mice is consistent with the hematopoietic expression of the Cre driver by the GFAP promoter (53) and the sensitivity of the hematopoietic lineage to LZTR1 mutations for tumor development in humans (3,54).

### Accumulation of EGFR and AXL in LZTR1-Mutant Schwannoma and Sensitivity to EGFR and AXL Inhibitors

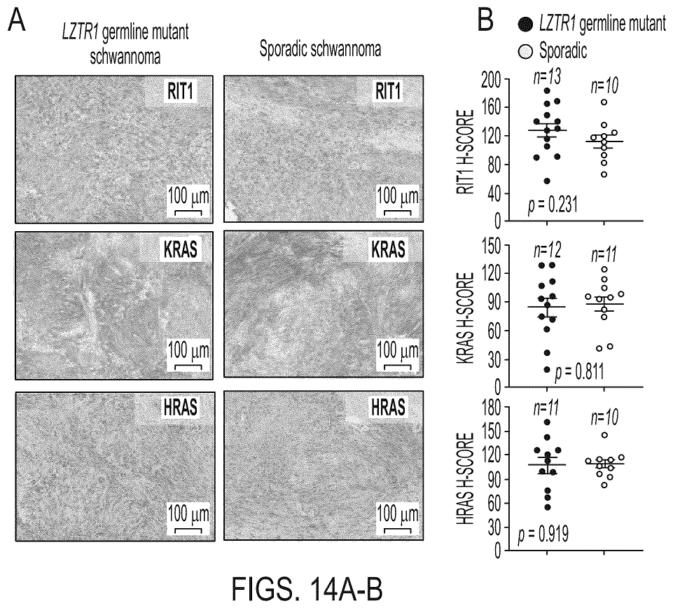

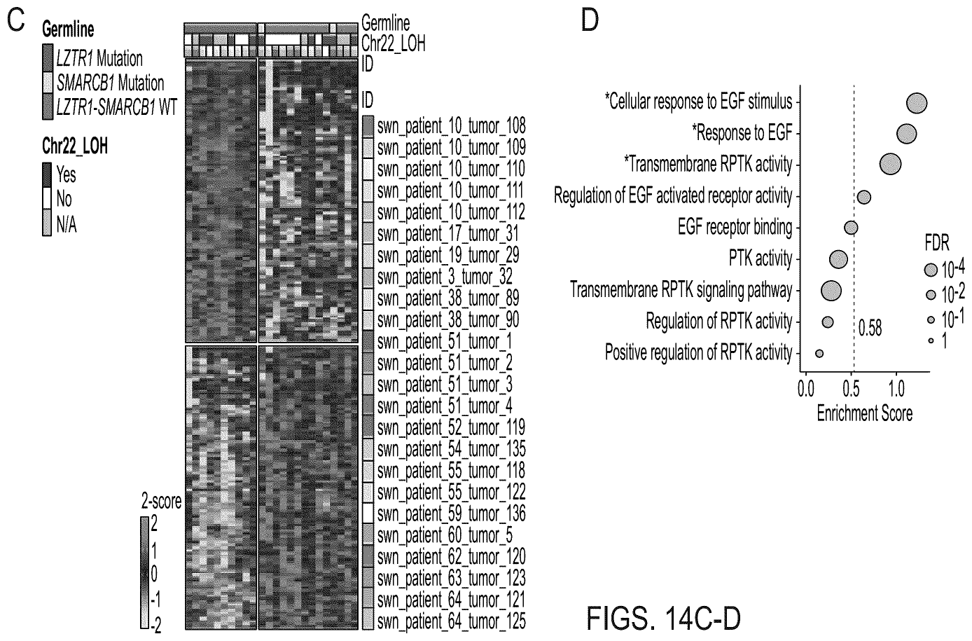

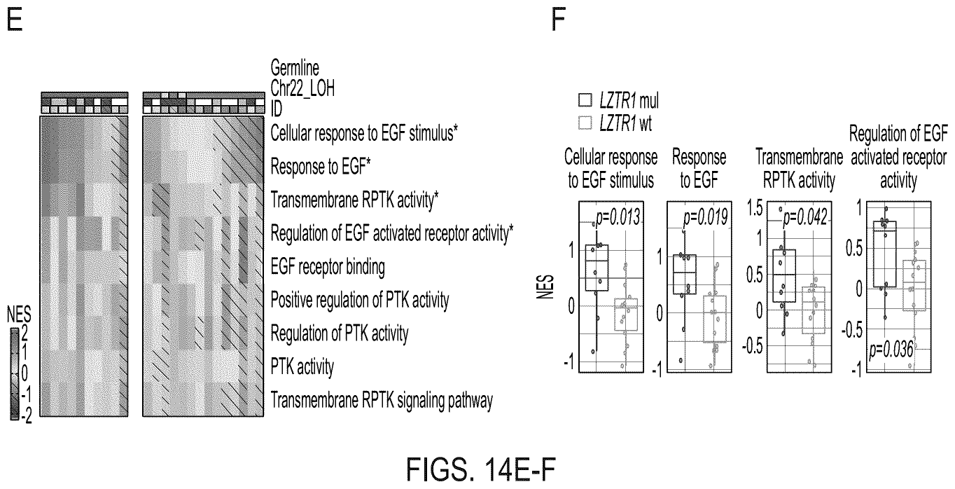

The above findings suggest that EGFR and AXL may aberrantly accumulate in human tumors initiated by inactivation of LZTR1. To test this hypothesis, EGFR and AXL protein levels were compared in schwannomas from individuals carrying germline mutations of LZTR1 and affected by Schwannomatosis, schwannomas from individuals carrying germline mutations of SMARCB1 and sporadic/non-syndromic schwannoma. EGFR and AXL proteins were analyzed by immunohistochemistry and quantified with the digital HSCORE (D-HSCORE), an accurate and reproducible method for quantitative immunostaining (55). EGFR and AXL accumulated in schwannomas from LZTR1 germline mutant Schwannomatosis patients at levels significantly higher than those detected in SMARCB1 mutant or sporadic schwannomas (FIG. 6G and H). However, expression of RIT1, KRAS and HRAS did not vary significantly between sporadic or LZTR1 mutant germ line schwannoma (FIG. 14A and B). To validate the finding that RTK signaling is enhanced in LZTR1 germ line mutant schwannoma compared with LZTR1 wild type Schwannomatosis tumors, the transcriptomic profiles were analyzed from RNA seq data of Schwannomatosis-schwannoma from patients with germline mutations of LZTR1 (n=10) and Schwannomatosis-schwannoma from patients without LZTR1 germline mutation (n=14) (56). By performing differential gene expression analysis of 10 schwannoma from LZTR1 germline mutant and 14 tumors from patients without LZTR1 germline mutation and the robust GST (Gene Set Test)-MWW (Mann-Whitney-Wilcoxon) test statistics (57), it was found that EGF-dependent RTK activation and RTK activities are significantly higher in tumors from patients with LZTR1 mutation compared with tumors harboring wild type LZTR1 (FIG. 14C and D). The independent analysis of single-sample enrichment score for EGFR and RTK gene signatures confirmed that response to EGF and RTK activity and regulation are the biological pathway significantly increased in tumors from patients with LZTR1 germline mutations (FIG. 14E and F). There was no significant difference in the occurrence of chromosome 22q loss of heterozygosity (LOH) between the two groups (Fisher's Exact Test p-value=1), suggesting that the RTK pathway activation is unlikely to be linked to this genetic event (FIG. 14C).

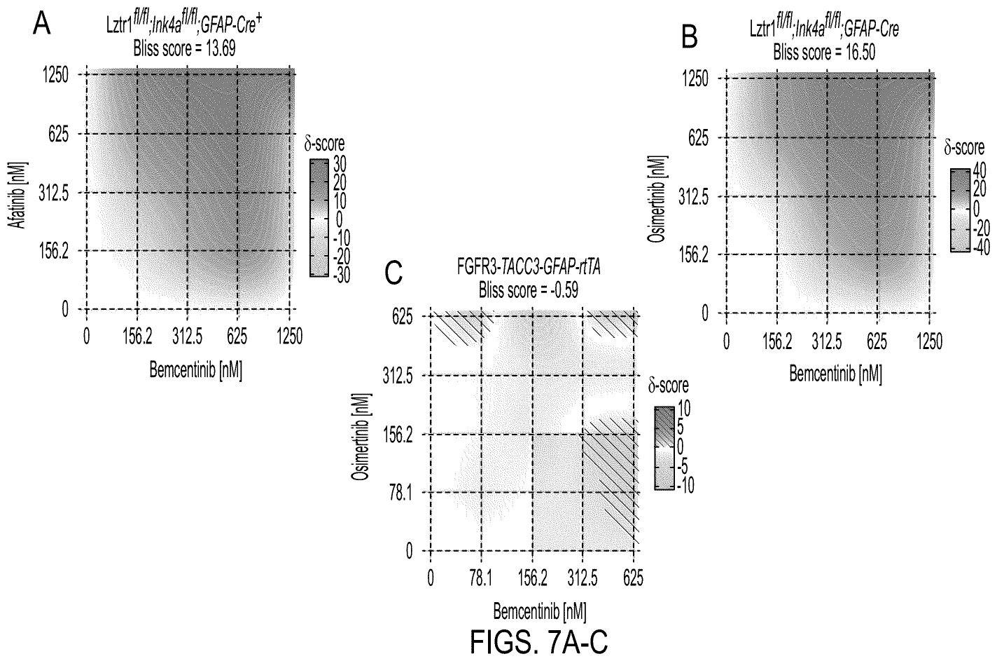

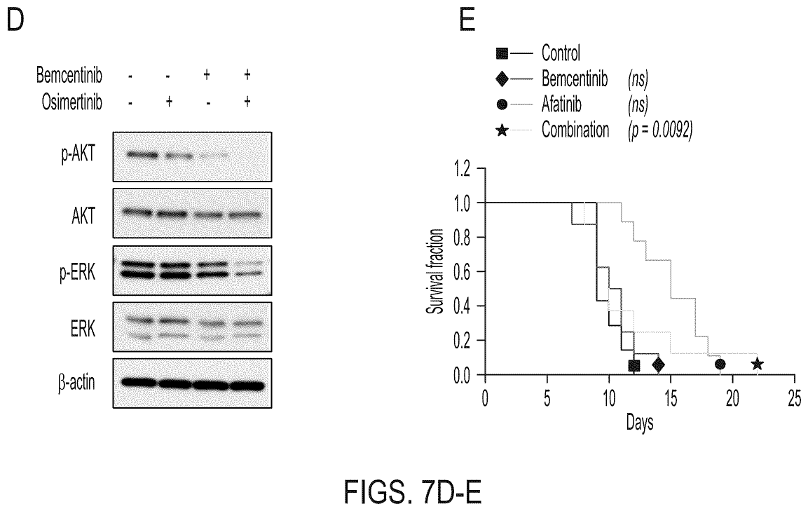

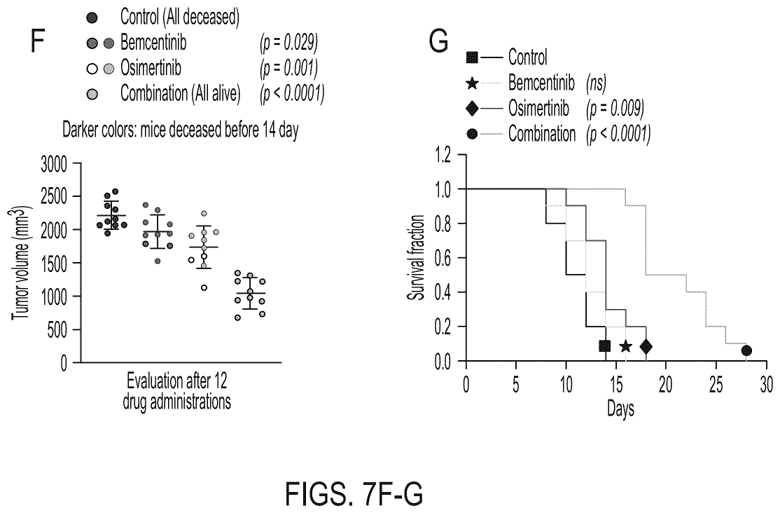

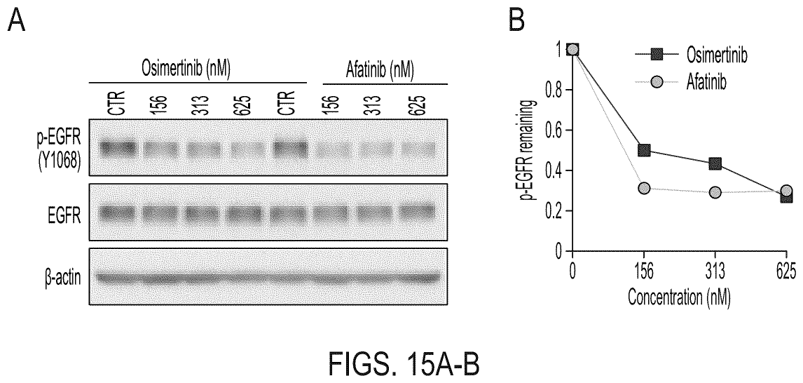

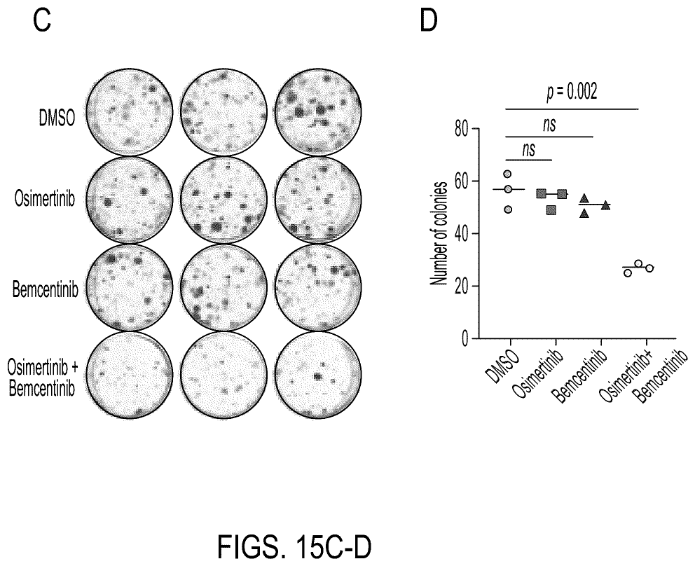

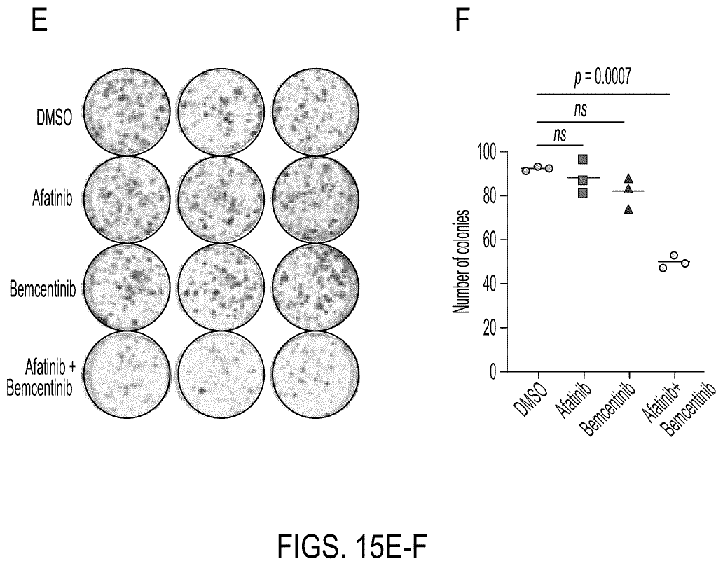

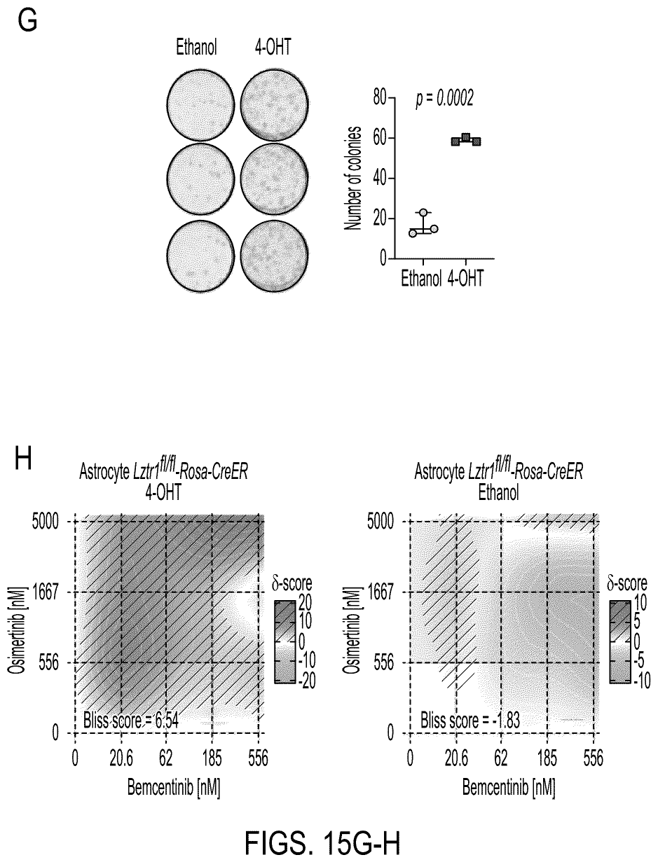

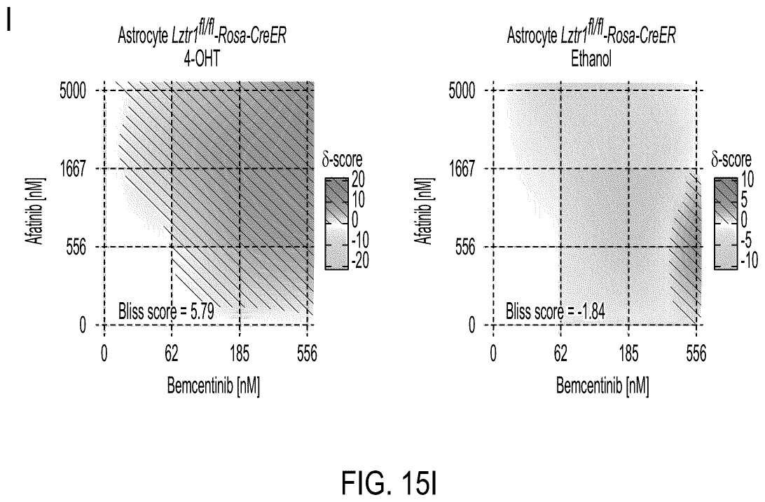

Finally, it was asked whether the accumulation of RTKs in cells harboring loss of LZTR1 affected the proliferation potential and induced vulnerability to inhibition of EGFR and AXL kinase activity. For these experiments two EGFR inhibitors, afatinib and osimertinib and the AXL specific inhibitor, bemcentinib (58) were considered. Afatinib is an irreversible pan-ErbB inhibitor that targets wild type EGFR (59). Osimertinib binds to the EGFR kinase irreversibly by targeting the cysteine-797 residue in the ATP binding site via covalent bond formation (60). Although the inhibitory activity of osimertinib against EGFR-T790M mutation is well established in lung cancer, the inhibition of the wild-type EGFR kinase is still considerable (61-64). To evaluate target engagement by the two EGFR inhibitors in cells isolated from a schwannoma-like mouse tumor (E0954), we analyzed the phosphorylation of EGFR after treatment with different concentrations of osimertinib and afatinib. Both inhibitors decreased phosphorylation of Tyr-1068 of EGFR although afatinib showed a more pronounced block than osimertinib at the lowest concentrations (FIG. 15A and B). When analyzed at a single dose for the effect on clonogenicity of schwannoma-like cells from Lztr1^{fl/fl};Cdkn2A^{fl/fl};GFAP-Cre+ mice (E0954), both afatinib and osimertinib as well as the AXL inhibitor bemcentinib were ineffective. However, treatment with each EGFR inhibitor in combination with bemcentinib severely reduced clonogenicity (FIG. 15C-F). To validate this result, the drug combination effects were determined at multiple doses using the Bliss score. The E0954 schwannoma-like cells exhibited high sensitivity to combination treatment with afatinib or osimertinib and bemcentinib (FIG. 7A and B; Bliss synergistic score: 13.69 and 16.5, respectively). In contrast, the combination of osimertinib plus bemcentinib lacked a synergistic effect in cancer cells derived from a mouse model of FGFR3-TACC3 gene fusion (57,65) (FIG. 7C; Bliss score: −0.59), thus supporting the specificity of therapeutic synergy. The molecular synergy of osimertinib and bemcentinib was evident in the inhibition of the downstream signaling, with complete elimination of AKT phosphorylation and significant reduction of ERK phosphorylation by the drug combination compared with treatment with the individual drugs (FIG. 7D). Confirmation of whether EGFR and AXL inhibitors have synergistic effects in immortalized astrocytes isolated from Lztr1^{fl/fl};Rosa-CreER mice either left untreated or treated with 4-OHT to delete the Lztr1 gene was also performed. Lztr1 deletion by 4-OHT treatment markedly increased colony formation (FIG. 15G). Recombined and control cells were treated with the EGFR inhibitor osimertinib or afatinib and the AXL inhibitor bemcentinib individually and in combination. Co-inhibition of EGFR and AXL with osimertinib or afatinib and bemcentinib even at nanomolar concentration enhanced the killing of cells lacking active LZTR1 (4-OHT-treated) without affecting the viability of wild type astrocytes (ethanol-treated) (FIG. 15H and I). Finally, the anti-tumor effects of EGFR and AXL inhibitors was examined in vivo. In these experiments afatinib and osimertinib were tested alone or in combination with bemcentinib. Mice were injected subcutaneously with schwannoma-like cells from Lztr1^{fl/fl};Cdkn2A^{fl/fl};GFAP-Cre+ mice (E0954) and treated with i) bemcentinib at 50 mg/kg body weight, osimertinib at 5 mg/kg body weight, bemcentinib at 50 mg/kg body weight plus osimertinib at 5 mg/kg body weight or DMSO as control; ii) afatinib at 10 mg/kg bodyweight, bemcentinib at 50 mg/kg body weight, bemcentinib at 50 mg/kg body weight plus afatinib at 10 mg/kg body weight or DMSO as control (5 days in/one day off for both treatment regimens). Survival was evaluated by the log rank test. For both therapy regimens, single drug treatments were indistinguishable from control (FIG. 7E-G). Combination treatment of afatinib and bemcentinib prolonged survival compared to single and vehicle treatment (FIG. 7E, p=0.0092). However, this treatment regimen was considerably toxic in the mouse and required significant dose reduction. The combination of osimertinib plus bemcentinib was more effective in reducing tumor growth and improving mouse survival compared with single drug treatment and controls (FIG. 7F and G, p<0.0001). Taken together, in vitro and in vivo experiments validated the combination of EGFR and AXL inhibitors for treatment of LZTR1 mutant cancer.

### Discussion

This study shows that LZTR1 is recruited to ligand-activated EGFR and AXL, which are directly ubiquitylated by CRL3^{LZTR1} complexes and consequently targeted to the lysosome for proteolysis. Thus, LZTR1 controls the timing of termination of the signal generated by the two RTKs. In the absence of LZTR1, EGFR and AXL remain in the endosome compartment and are likely recycled to the plasma membrane. Remarkably, most LZTR1 mutations of Kelch and BTB-BACK domain found in different sporadic cancer types and the germline of patients with Schwannomatosis have lost the ability to destabilize EGFR and AXL. The effect of Kelch domain mutants is associated with loss of the interaction between the LZTR1 mutants and the RTK substrates whereas the mutants in the BTB-BACK domains specifically impair the formation of complex with CUL3 (66). Consistent with these findings, EGFR and AXL specifically accumulate in tumors from LZTR1-mutant Schwannomatosis, resulting in deregulated downstream signaling. It was also found that schwannoma-like tumor cells lacking the LZTR1 gene exhibited vulnerability to combinatorial inhibition of EGFR and AXL. This finding was confirmed in multiple cell systems in vitro and in vivo and was similarly reproduced when using different small molecules inhibitors of EGFR.

The analysis of the Lztr1 deficient mouse model showed that constitutive homozygous deletion of Lztr1 is embryonically lethal at mid-gestation because of severe liver degeneration. Knockout embryos also exhibited expansion of the germinal areas of the telencephalon with reduction of the differentiated cortical plate. Thus, the phenotypes induced by Lztr1 deficiency in the mouse differ from the pathological traits that have been reported in mice harboring gain of function mutation of the LZTR1 substrate RIT1, which recapitulate the dysmorphic alterations in Noonan syndrome (20). Interestingly, conditional knockout of Lztr1 and Cdkn2a under the GFAP promoter, which is widely active in progenitor and glial cells in the CNS and PNS did not trigger tumorigenesis in the brain but induced PNS tumors including schwannoma-like tumors and MPNST and generated sensitivity to developing myeloid neoplasms. These results demonstrated for the first time the tumor suppression function of LZTR1 in the whole organism in vivo and unmasked the striking sensitivity of cells of the PNS to neoplastic transformation by LZTR1 inactivation. Deregulation of EGFR and AXL signaling in LZTR1-mutant cells points to a broad level of complexity of the control of cell growth and survival since RTK s are also coupled to non-Ras-dependent pathways (67,68). EGFR and AXL are among the most studied tumor-specific RTK s undergoing genetic alterations that generate oncogenic signals driving multiple cancer types. However, beside copy number gain or activating mutations, post-translational accumulation of the EGFR and AXL proteins is frequently observed in the absence of gene amplification (69-71). Furthermore, ligand-induced activation of these RTK s is a potent oncogenic mechanism in diverse cancer types (72-74). The frequent loss-of-function alterations of the LZTR1 gene across multiple tumor types and in tumor predisposition syndromes increase RTK protein stability and may promote tumor initiation and progression by activating distinct cancer hallmarks such as epithelial to mesenchymal transition and invasion (75,76). By targeting at once EGFR and AXL for degradation, LZTR1 not only restrains EGFR but also blocks the parallel and redundant cancer signaling mediated by AXL. EGFR and AXL can be directly targeted by small molecule inhibitors. The therapeutic effect of single treatment with the EGFR inhibitors afatinib and osimertinib or the AXL inhibitor bemcentinib was minimal. However, when used in combination, EGFR and AXL inhibitors exhibited specific anti-tumor effect against LZTR1-deficient immortalized astrocytes, PNS tumor cells, and tumor models in vivo, indicating that when treated with single RTK inhibitor, the persistent activity of EGFR or AXL is sufficient to sustain survival and growth of LZTR1-mutant cells. Overall, the data can be safely interpreted to indicate that the combinatorial inhibition of EGFR and AXL represents a therapeutic opportunity for LZTR1 mutant tumors.

### Methods

### Cell Culture

HeLa (ATCC, CCL-2, RRID:CVCL0030), HEK 293T (ATCC, CRL-11268, RRID:CVCL_1926), SW10 (ATCC, CRL-2766, RRID:CVCL_6458) and U87-MG (HTB-14, RRID:CVCL_0022) cell lines were acquired through American Type Culture Collection. Cell lines were cultured in DMEM supplemented with 10% fetal bovine serum (FBS, Sigma). Cells were routinely tested for Mycoplasma contamination using Mycoplasma Plus PCR Primer Set (Agilent, Santa Clara, CA) and were found to be negative. Mouse embryonic fibroblasts were isolated from embryonic day 13.5 (E13.5)-old LZTR1+/+ and LZTR1−/− embryos and astrocytes LZTR1^{fl/fl}-Rosa-CreER were isolated from post-natal day 5 pups according to published protocols (77,78). Deletion of Lztr1 in astrocytes was achieved by treating cells with 350 nM 4-hydroxytamoxifen for 4 days. EGF and epiregulin stimulation was performed by adding the ligand at the concentrations indicated in the individual figures after 16 hours of starvation from serum. Cells were transfected with Lipofectamine 2000 (Invitrogen) or calcium phosphate. Cells were transduced using lentiviral particles in medium containing 4 μg/ml of polybrene (Sigma). Evaluation of protein half-life was performed by treating cells with 50 μg/ml of cycloheximide (CHX) for the indicated times and analyzed by western blot. The half-life of EGFR and AXL was quantified by densitometry using ImageJ processing software (NIH, RRID:SCR_003070). Densitometry values were analyzed by Prism 6.0 (RRID:SCR_002798).

### Plasmids, Cloning and Lentivirus Production

CDNAs for LZTR1 and AXL were amplified by PCR and cloned into the pCDNA3.1 or pLOC vectors in frame with FLAG or HA tag at the C-terminus. Expression of LZTR1 using a doxycycline inducible system was achieved by cloning FLAG-tagged LZTR1 DNA into pINDUCER-TR3G. EGFR-GFP was a gift from Alexander Sorkin (Addgene plasmid #32751, RRID:Addgene_32751). GST-LZTR1 full length and deletion mutants (LZTR1-1-425, LZTR1-426-840, LZTR1-426-640, LZTR1-641-840) and GST-EGFR deletion mutants (EGFR-1-620, EGFR-643-953, EGFR-643-1186, EGFR-953-1186) were cloned into pGEX-4T-3 and resulting plasmids verified by Sanger sequencing. EGFR kinase activity defective mutant, K721R and LZTR1 Kelch domain mutants were generated by site-directed mutagenesis using the QuickChange Site-Directed mutagenesis kit (Agilent) and verified by Sanger sequencing.

For CRISPR/Cas9-mediated LZTR1, we used lentiviral vector (LV01, Sigma). sgRNA sequences are:
control non-targeting: CGCGATAGCGCGAATATATT (SEQ ID NO: 1)
hLZTR1-1: CACCCACGAACTCGTCGCA (SEQ ID NO: 2) (HSPD0000047303);
hLZTR1-2: GACTTCGACCATAGCTGCT (SEQ ID NO: 3) (HSPD0000047304);
mLZTR1-1: CATGGAAGAGCCTCCCGCT (SEQ ID NO: 4) (MMPD0000065735);
mLZTR1-2: GACAACAACATTCGCAGTG (SEQ ID NO: 5) (MMPD0000065733).

Lentiviral particles were produced by co-transfecting of lentiviral vectors with pCMV-ΔR8.1 and pMD2.G plasmids into HEK 293T cells as previously described (79). After lentiviral infection, cells were selected with puromycin, seeded in a 96-well plate at a density of 0.5 cell per well and independent clones isolated, expanded, and analyzed by western blot for expression of the targeted genes.

### Global Ubiquitylation Enrichment

For global ubiquitylation enrichment study, immortalized LZTR1fl/fl-Rosa-CreER astrocytes were treated with 4-OHT as described above to induce deletion of LZTR1. Cells were then infected with lentivirus for doxycycline inducible reconstitution of LZTR1 and treated with doxycycline or vehicle to express LZTR1 in the absence or presence of MG132. Cells were lysed/homogenized by bead-beating in 9 M urea and 100 mM ammonium bicarbonate, supplemented with protease inhibitors. Lysates were cleared by centrifugation at 21,000 g for 30 min at 40 C, and protein concentration was measured by BCA. Five mg of total protein from each sample were reduced with 10 mM DTT for 30 min at 56° C. in an air thermostat, cooled down to room temperature, and alkylated with 11 mM CAA at room temperature for 30 min, and alkylation was then quenched by the addition of an additional 5 mM DTT. Samples were diluted 6-fold with 50 mM ammonium bicarbonate and digested overnight with trypsin (1:50) at 37° C. The next day, the digestion was stopped by the addition of 0.25% TFA (final v/v) and centrifuged at 10,000 rpm for 10 min at room temperature to pellet precipitated lipids and collected the cleared supernatant. Supernatant were desalted on a SepPak C18 cartridge 500 mg and dried using vacuum centrifugation. Desalted dried peptides (5 mg) were resuspended in 1.4 ml of ice-cold IAP buffer (50 mM MOPS (pH 7.2), 10 mM sodium phosphate and 50 mM NaCl) and centrifuged at maximum speed for 5 min at 4° C. to remove any insoluble material. Supernatants (pH˜7.5) were incubated with the washed PT M Scan® Ubiquitin Remnant Motif (K-ε-GG) Kit #5562 (Cell Signaling Technology) antibody beads for 2 hours at 4° C. After centrifugation at 2000 g for 1 min, beads were washed three times with ice-cold IAP buffer and three with ice-cold HPLC water. The ubiquitinated peptides were eluted twice with 0.15% TFA, desalted using SDB-RP StageTip, and dried via vacuum centrifugation. Desalted peptides were injected in an EASY-Spray™ PepMap™ RSLC C18 50 cm×75 cm ID column (Thermo Scientific) connected to an Orbitrap Fusion™ Tribrid™ (Thermo Scientific). Peptides elution and separation were achieved at a non-linear flow rate of 250 nl/min using a gradient of 5%-30% of buffer B (0.1% (v/v) formic acid, 100% acetonitrile) for 110 min with a temperature of the column maintained at 50° C. during the entire experiment. The Thermo Scientific Orbitrap Fusion Tribrid mass spectrometer was used for peptide tandem mass spectroscopy (MS/MS). Survey scans of peptide precursors are performed from 375 to 1500 m/z at 120K full width at half maximum (FWHM) resolution (200 m/z) with a 2×105 ion count target and a maximum injection time of 50 ms. The instrument was set to run in top speed mode with 3-second cycles for the survey and the MS/MS scans. After a survey scan, MS/MS was performed on the most abundant precursors, i.e., those exhibiting a charge state from 2 to 6 of greater than 5×103 intensity, by isolating them in the quadrupole at 1.6 Th. We used collision-induced dissociation (CID) with 35% collision energy and detected the resulting fragments with the rapid scan rate in the ion trap. The automatic gain control (AGC) target for MS/MS was set to 1×104 and the maximum injection time was limited to 35 ms. The dynamic exclusion was set to 15 s with a 10 ppm mass tolerance around the precursor and its isotopes. Monoisotopic precursor selection was enabled. Raw mass spectrometric data were analyzed using the MaxQuant environment v.1.6.1.0 (80) and Andromeda for database searches (81) at default settings with few modifications. The default was used for first search tolerance and main search tolerance (20 ppm and 6 ppm, respectively). MaxQuant was set up to search with the reference mouse proteome database downloaded from UniProt. MaxQuant and performed the search trypsin digestion with up to 2 missed cleavages. Peptide, site and protein false discovery rates (FDR) were all set to 1%. The following modifications were used for protein identification and quantification: oxidation of methionine (M), acetylation of the protein N-terminus, DiGly (K) and deamination for asparagine or glutamine (NQ). Results obtained from MaxQuant were filtered by localization probability of ubiquitination site >0.90; intensity of diGly peptide >median; summed score for the individual peptides >15th percentile; posterior error probability <0.05; intensity fold change between samples with expression of LZTR1 and control >2.5.

Tandem Mass Tag (TMT) quantitative mass spectrometry

To identify the proteins that accumulated following LZTR1 loss, immortalized LZTR1 wild type and LZTR1−/− MEFs or Hela cells were collected in cold PBS and centrifuged at 4,000 RPM for 5 min and processed as described (82). Briefly, frozen cell pellets were lysed by bead-beating in 8 M urea and 200 mM EPPS (pH 8.5), supplemented with protease inhibitors. Samples were reduced with 5 mM TCEP and alkylated with 10 mM iodoacetamide (IAA) that was quenched with 10 mM DTT. A total of 200 μg of protein was chloroform-methanol precipitated. Protein was reconstituted in 200 mM EPPS (pH 8.5) and digested by Lys-C overnight and trypsin for 6h, both at a 1:50 protease-to-peptide ratio. Digested peptides were quantified using a Nanodrop and 100 μg from each sample were labeled with 800 μg TMT reagent using 10-plex TMT kit1. TMT labels were checked, 0.5 μg of each sample was pooled, desalted, and analyzed by short SPS-MS3 method, and using normalization factor, samples were bulk mixed at 1:1 across all channels. 900 μg of the bulk mixed sample was used for total proteome analysis. Mixed™ T-labeled samples were vacuum centrifuged and desalted with C18 Sep-Pak (200 mg) solid-phase extraction column. The desalted sample was fractionated using BPRP chromatography. Peptides were subjected to a 50 min linear gradient from 5 to 42% acetonitrile in 10 mM ammonium bicarbonate pH 8 at a flow rate of 0.6 mL/min over Water X-bridge C18 column (3.5 μm particles, 4.6 mm ID and 250 mm in length). The peptide mixture was fractionated into a total of 96 fractions, which were consolidated into 36 fractions. Fractions were subsequently acidified with 1% formic acid, and vacuum centrifuged to near dryness and desalted via SDB-RP StageTip. Fractions were dissolved in 10 μl of 3% acetonitrile/0.1% formic acid injected using SPS-MS3. The UltiMate 3000 UHPLC system (Thermo Scientific) and EASY-Spray PepMap RSLC C18 50 cm×75 μm ID column (Thermo Fisher Scientific) coupled with Orbitrap Fusion (Thermo) were used to separate fractioned peptides with a 5-30% acetonitrile gradient in 0.1% formic acid over 45 min at a flow rate of 250 nL/min. After each gradient, the column was washed with 90% buffer B for 10 min and re-equilibrated with 98% buffer A (0.1% formic acid, 100% HPLC-grade water) for 40 min. The full MS spectra were acquired in the Orbitrap Fusion™ Tribrid™ Mass Spectrometer (Thermo Fisher Scientific) at a resolution of 120,000. The 10 most intense MS1 ions were selected for MS2 analysis. The isolation width was set at 0.7 Da and isolated precursors were fragmented by CID at normalized collision energy (NCE) of 35% and analyzed in the ion trap using “turbo” scan speed. Following the acquisition of each MS2 spectrum, a synchronous precursor selection (SPS) MS3 scan was collected on the top 10 most intense ions in the MS2 spectrum. SPS-MS3 precursors were fragmented by higher energy collision-induced dissociation (HCD) at an NCE of 65% and analyzed using the Orbitrap. Raw mass spectrometric data were analyzed using Proteome Discoverer 2.3 to perform database search and TMT reporter ions quantification. TMT tags on lysine residues and peptide N termini (+229.163 Da) and the carbamidomethylation of cysteine residues (+57.021 Da) was set as static modifications, while the oxidation of methionine residues (+15.995 Da), and deamidation (+0.984) on asparagine and glutamine were set as a variable modification. Data were searched against a UniProt Mouse or human database with peptide-spectrum match (PSM s) and protein-level FDR at 1% FDR. The signal-to-noise (S/N) measurements of each protein was normalized so that the sum of the signal for all proteins in each channel was equivalent to account for equal protein loading. Protein identification and quantification were analyzed using the R software environment. Proteins were selected according to >1.5-fold change between LZTR1 knock out and wild type samples.

### Identification of LZTR1 Interacting Proteins by Mass Spectrometry.

LZTR1 complexes were purified from the U87 cells transduced with lentivirus expressing LZTR1-FLAG, LZTR1-FLAG-R810W or the empty vector. Cellular lysates were prepared in 50 mM Tris-HCl, pH 7.5, 150 mM NaCl, 1 mM EDTA, 1% NP40, 0.5% sodium deoxycholate, 0.1% sodium dodecyl sulphate, 1.5 mM Na3VO4, 50 mM sodium fluoride, 10 mM sodium pyrophosphate, 10 mM β-glycerolphosphate and EDTA-free protease inhibitor cocktail (Roche). Lysates were immunoprecipitated with FLAG affinity matrix and eluted with FLAG peptide. Eluates were separated on 4-12% gradient SDS-PAGE and stained with SimplyBlue (Thermo fisher Scientific). Protein gel slices were excised and in-gel digestion performed as previously described (83), with minor modifications. Gel slices were washed with 1:1 A cetonitrile and 100 mM ammonium bicarbonate for 30 min then dehydrated with 100% acetonitrile for 10 min until shrunk. The excess acetonitrile was then removed and the slices dried in speed-vacuum at room temperature for 10 min. Gel slices were reduced with 5 mM DTT for 30 min at 56° C. in an air thermostat, cooled down to room temperature, and alkylated with 11 mM IAA for 30 min with no light. Gel slices were then washed with 100 mM of ammonium bicarbonate and 100% acetonitrile for 10 min each. Excess acetonitrile was removed and dried in a speed-vacuum for 10 min at room temperature and the gel slices were re-hydrated in a solution of 25 ng/μl trypsin in 50 mM ammonium bicarbonate for 30 min on ice and digested overnight at 37° C. in an air thermostat. Digested peptides were collected and further extracted from gel slices in extraction buffer (1:2 ratio by volume of 5% formic acid:acetonitrile) at high speed, shaking in an air thermostat. The supernatants from both extractions were combined and dried in a speed-vacuum. Peptides were dissolved in 3% acetonitrile/0.1% formic acid. Desalted peptides were injected in an EASY-Spray™ PepMap™ RSLC C18 50 cm×75 cm ID column (Thermo Scientific) connected to an Orbitrap Fusion™ Tribrid™ (Thermo Scientific) as described in “Global ubiquitylation enrichment” section. The following modifications were used for protein identification and quantification: oxidation of methionine (M), acetylation of the protein N-terminus, and deamination for asparagine or glutamine (NQ). Results obtained from MaxQuant were assembled in Scaffold for data visualization. A specificity score of proteins interacting with LZTR1 proteins was computed for each polypeptide as described (84). Briefly, we compared the number of peptides identified from our mass spectrometry analysis to those reported in the CRAPome database that includes a list of potential contaminants from affinity purification-mass spectrometry experiments (www.crapome.org). The specificity score is computed as [(#peptide*#xcorr)/(AveSC*MaxSC*#of Expt.)], where #peptide, identified peptide count; #xcorr, the cross-correlation score for all candidate peptides queried from the database; AveSC, averaged spectral counts from CRAPome; MaxSC, maximal spectral counts from CRAPome; and #of Expt., the total found number of experiments from CRAPome. Finally, proteins were filtered by Peptide Spectrum Matches (PSM)=0 in the empty vector (EV) control; PSM>0 in the cells expressing wild type (WT) and R810W mutant (RW) LZTR1; RW-LZTR1 vs. WT-LZTR1 PSM fold change >2.

### Recombinant Protein Production and GST Pull Down Assay

PGEX 4T-3-EGFR and PGEX 4T-3-LZTR1 full length or deletion mutant plasmids were introduced into E. coli strain BL21 (DE3). Protein expression was induced with 200 μM isopropyl-β-D-1-thiogalactopyranoside (IPTG) for 4-5 hours at 30° C. Bacteria were harvested and resuspended in lysis buffer containing 1× phosphate buffered saline, 0.5% Triton-X 100, 1× protease inhibitors cocktail (Roche), 1 mM PMSF, and 200 μg/mL lysozyme and incubated on ice for 30 min. After centrifugation at 17,000 g for 15 min at 4° C., supernatants were incubated with Glutathione S Sepharose beads (Cytiva) for 1 hour at 4° C., washed 3 times in lysis buffer and stored in PBS.

### Immunoblot and Immunoprecipitation

Cells were lysed in RIPA buffer (50 mM Tris-HCl, pH 7.5, 150 mM NaCl, 1 mM EDTA, 1% NP40, 0.5% sodium deoxycholate, 0.1% sodium dodecyl sulphate, 1.5 mM Na3VO4, 50 mM sodium fluoride, 10 mM sodium pyrophosphate, 10 mM β-glycerolphosphate and EDTA-free protease inhibitor cocktail (Roche)) or NP40 lysis buffer (50 mM Tris-HCl, pH 7.5, 150 mM NaCl, 1 mM EDTA, 1% NP40, 1.5 mM Na3VO4, 50 mM sodium fluoride, 10 mM sodium pyrophosphate, 10 mM β-glycerolphosphate and EDTA-free protease inhibitor cocktail (Roche)). Lysates were centrifuged 14,000 r.p.m. for 15 min at 4° C. and supernatant were collected. For immunoprecipitation, cell lysates were incubated with antibody for GFP (Santa Cruz Biotechnology Cat #sc-9996, RRID:AB_627695) or EGFR (Santa Cruz Biotechnology Cat #sc-373746, RRID:AB_10920395) and protein G/A beads (Santa Cruz Biotechnology Cat #sc-2003, RRID:AB_10201400) or Flag M2 affinity gel (Sigma-Aldrich Cat #F2426, RRID:AB_2616449) and HA affinity gel (Sigma-Aldrich Cat #E6779, RRID:AB_10109562) at 4° C. overnight. Beads were washed with lysis buffer four times and eluted in 2×SDS sample buffer. Immunoprecipitates or lysates were separated by SDS-PAGE and transferred to polyvinylidene difluoride or nitrocellulose membranes. Membranes were blocked in TBS-T (0.1% Tween20) with 5% non-fat milk or BSA and probed with primary antibodies.

Antibodies and working concentrations are: GFP (1:1000; Santa Cruz Biotechnology Cat #sc-9996, RRID:AB_627695); LZTR1 (1:500; Santa Cruz Biotechnology Cat #sc-390166, RRID:AB_2910196); EGFR (1:500; Santa Cruz Biotechnology Cat #sc-373746, RRID:AB_10920395); AXL (1:1000; Santa Cruz Biotechnology Cat #sc-166269, RRID:AB_2243305); c-MYC (1:500; Santa Cruz Biotechnology Cat #sc-40, RRID:AB_627268); GST (1:1000; Santa Cruz Biotechnology Cat #sc-138, RRID:AB_627677); ERK (1:2000; Cell Signaling Technology Cat #9102, RRID:AB_330744); phospho-ERK (1:2000; Cell Signaling Technology Cat #4370, RRID:AB_2315112); phospho-EGFR (1:1000; Cell Signaling Technology Cat #3777, RRID:AB_2096270); AXL (1:2000; Cell Signaling Technology Cat #8661, RRID:AB_11217435); phosphor-AXL (1:500; Cell Signaling Technology Cat #5724, RRID:AB_10544794); DYKDDDDK Tag (1:1000; Cell Signaling Technology Cat #14793, RRID:AB_2572291); β-actin (1:10000; Sigma-Aldrich Cat #A5441, RRID:AB_476744); Mono- and polyubiquitinilated-HRP (1:1000; Enzo Life Sciences Cat #BML-PW0150-0025, RRID:AB_2051892); EGFR (1:2000; Abcam Cat #ab52894, RRID:AB_869579); RIT1 (1:500; Abcam Cat #ab53720, RRID:AB_882379); HA (1:1000; Roche Cat #12158167001, RRID:AB_390915); AXL (1:1000; R and D Systems Cat #AF854, RRID:AB_355663). Horseradish peroxidase-conjugated secondary antibodies were purchased from Invitrogen and ECL (Amersham) or Super Signal West Femto (Thermo Scientific) was used for detection.

### Ubiquitylation Assay

293T and Hela cells were transfected with plasmid expressing EGFR-GFP, HA-Ub and LZTR1-FLAG as indicated in figures and treated with MLN4924 (1 μM), chloroquine (CQ, 100 μM), MG132 (20 μM), or EGF (20 ng/ml or 100 ng/ml) and ubiquitylation assay was performed under denaturing condition. Cells were lysed in 1% SDS and boiled at 100° C. for 10 min. Lysates were diluted with tris buffered saline containing 1% N P40 and centrifuged 14000 r.p.m. for 15 min at 4° C. Immunoprecipitation was performed using 500 μg to 1 mg of cellular lysates using HA affinity gel (Sigma, E6779), AXL or EGFR antibodies at 4° C. overnight followed by protein G/A beads for 90 min at 4° C. Beads were washed with lysis buffer four times and eluted in 2×SDS sample buffer. Protein samples were separated by SDS-PAGE and transferred to PV DF membrane and analyzed by western blot.

### Immunofluorescence and Immunohistochemistry

Cells were fixed with 4% paraformaldehyde for 10 min, washed with cold PBS three times and permeabilized and blocked with 0.02% saponin or 0.5% Triton X 100 and 5% BSA in PBS for 1 hour. Cells were incubated with antibodies; EEA1 antibody (1:200; BD Biosciences Cat #610457, RRID:AB_397830); Lamp1 antibody (1:500; Abcam Cat #ab24170, RRID:AB_775978); Lamp1 (1:700; Santa Cruz Biotechnology Cat #sc-20011, RRID:AB_626853); AXL (1:1000; Cell Signaling Technology Cat #8661, RID: AB_11217435); AXL (1:400; Santa Cruz Biotechnology Cat #sc-166269 RRID:AB_2243305); FLAG (1:1000; Sigma-Aldrich Cat #F1804, RRID:AB_262044) overnight at 4° C. or 1 hr at RT. Cells were washed with 0.02% Saponin in PBS for three times and incubated with fluorescence-conjugated secondary antibodies; Goat anti-Rabbit IgG (H+L) Cross-Adsorbed Secondary Antibody, Cyanine3 (Thermo Fisher Scientific Cat #A10520, RRID:AB_253402); Cy™3 AffiniPure Donkey Anti-Goat IgG (H+L) (Jackson ImmunoResearch Labs Cat #705-165-147, RRID:AB_2307351); Goat anti-Mouse IgG (H+L), Superclonal Recombinant Secondary Antibody, Alexa Fluor™ 555 (Thermo Fisher Scientific Cat #A28180, RRID:AB_2536164); Goat anti-Rabbit IgG (H+L) Highly Cross-Adsorbed Secondary Antibody, Alexa Fluor™ 647 (Thermo Fisher Scientific Cat #A-21245, RRID:AB_2535813); Goat anti-Mouse IgG (H+L) Cross-Adsorbed Secondary Antibody, Alexa Fluor™ 647 (Thermo Fisher Scientific Cat #A-21235, RRID:AB_2535804) for 30 min at RT. After three washes, DNA was counterstained with DAPI (Sigma). Fluorescence microscopy was performed on a Nikon A1R MP microscope using a 100×, 1.45 Plan Apo Lambda lens or Olympus IX70 microscope with 40× objective. Image analysis was performed using ImageJ (RRID:SCR_003070). At least 30 cells per sample (4-10 cells per field) were randomly selected and co-localization between EGF or AXL and EEA1 or Lamp1 quantified using the ImageJ plugin JACoP. Human and mouse tissue preparation and immunostaining were performed as previously described (84,85). Human schwannoma samples analyzed by immunohistochemistry had been stored in the Onconeurotek tumorbank (certified NF S96 900), and received the authorization for analysis from ethical committee (CPP Ile de France VI, ref A39II), and French Ministry for research (AC 2013-1962). Briefly, tumor sections were deparaffinized in xylene and rehydrated in a graded series of ethyl alcohol. Antigen retrieval was performed in citrate solution pH=6.0 using decloaking chamber. After peroxidase blocking in 3% H2O2 for 15 min, slides were blocked for 1h in 10% goat serum, 0.25% Triton X-100, 1×PBS and then incubated at 4° C. overnight with antibodies; EGFR antibody (1:2500; Abcam Cat #ab52894, RRID:AB_869579); AXL antibody (1:250; Cell Signaling Technology Cat #8661, RRID:AB_11217435); RIT1 (1:500; Abcam Cat #ab53720, RRID:AB_882379); KRAS (1:800; Proteintech Cat #12063-1-AP, RRID:AB_878040); HRAS (1:150; Novus biologicals Cat #NBP2-42864); S100B (1:400; Abcam Cat #ab52642, RRID:AB_882426); Calretinin (1:400; Abcam, ab244299); SOX10 (1:500; Abcam, ab227680); AXL (1:2,000; R and D Systems Cat #AF854, RRID:AB_355663). Sections were then incubated with biotinylated anti-rabbit or anti-mouse antibody followed by streptavidin-peroxidase. Reaction for schwannomas tissue sections was developed by 3,3′-diaminobenzidine (DAB) and counterstained with hematoxylin. Images were acquired under 10× magnification using an Olympus 1×70 microscope equipped with digital camera section (6-25 images/section). Quantification of digital H-SCORE was obtained using ImageJ (NIH, Bethesda, USA) with specific built-in color deconvolution plugin for evaluation of hematoxylin and DAB staining (55). For mouse tumor immunofluorescence, TSA-Cy3 was used (Akoya Biosciences) and nuclei were counterstained with DAPI (Sigma). Semi-quantitative immunohistochemical analysis of PNS tumors was performed by scoring cells with positive signal for each marker according to the estimated percentage of positive cells: +++: positive cells ≥50% (diffuse); ++: positive cells between 25% and 50% (diffuse); +: positive cells between 10% and 25% (diffuse); +/−: positive cells between 10% and 25% (only in some fields with other fields negative); −: negative.

### Mouse Experiments

We obtained heterozygous Lztr1tm1a (EUCOMM) Wtsi mice through the European Community Mouse Mutagenesis consortium (36,86). Lztr1tm1a heterozygous mice are phenotypically normal. The Lztr1-deficient mice (LZTR1tm1a (EUCOMM) Wtsi) carry a knockout-first allele, in which a cassette including LacZ and neo genes were inserted at position 17518027 of Chr 16 (intron 5-6) of the Lztr1 gene. The cassette includes an FRT site followed by lacZ sequence and a loxP site. This first loxP site is followed by neomycin under the control of the human beta-actin promoter, SV40 polyA, a second FRT site and a second loxP site. A third loxP site is inserted downstream of the targeted exon 7 at position 17518811. LoxP sites thus flank exon 7. We generated the “conditional” (floxed) allele by inter-crossing them with ROSA 26Sortm1 (FLP1) mice. Lztr1^{fl/fl} mice were then intercrossed with Cdkn2a^{fl/fl} and GFAP-Cre mice to generate Lztr1^{fl/fl};Cdkn2a^{fl/fl};GFAP-Cre+ mice or with Rosa-cre-ER mice to obtain Lztr1^{fl/fl};Rosa-cre-ER mice from which astrocytes were isolated.

For in vivo treatment experiments, 1×106 of tumor cells from Lztr1^{fl/fl};Cdkn2a^{fl/fl};GFAP-Cre+ mice were injected subcutaneously in the right flank of females and males Nu/nu mice in 150 ul volume of cell cultured media with 50% Matrigel. Mice carrying 250-300 mm3 subcutaneous tumors (approximately 16 days from injection) were treated with i) bemcentinib at 50 mg/kg body weight, osimertinib at 5 mg/kg body weight, bemcentinib at 50 mg/kg body weight plus osimertinib at 5 mg/kg body weight or DMSO as control; ii) afatinib at 10 mg/kg bodyweight, bemcentinib at 50 mg/kg body weight, bemcentinib at 50 mg/kg body weight plus afatinib at 10 mg/kg body weight or DMSO as control (5 days in/one day off for both treatment regimens). Mice were weighted daily. Tumor diameters were measured every 2 days with caliper and tumor volumes estimated using the formula: width2×length/2=V (mm3). Mice were euthanized when the tumor size reached 2,000 mm3 or if the skin was ulcerated, according to IACUC recommendation. All experiments were performed according to the guidelines of the Institutional Animal Care and Use Committee at Columbia University.

### Colony Forming Assay

Astrocytes were treated with 350 nM 4-hydroxytamoxifen or ethanol for 4 days. 1,000 cells were plated in 6 well plates and cultured in DMEM/F12 medium supplemented with N-2, B-27, EGF and FGF-2 for 2 weeks. Cells were stained with crystal violet and colonies were counted.

### Clonogenic Assay

500 cells were seeded in triplicate wells of 6-well plates. 24 hr after seeding, cells were treated with osimertinib, afatinib and bemcentinib for 72 hr as indicated in FIG. 15C and E. Cells were washed to remove the drugs and cultured in growth culture medium until colonies were detected by light microscopy. Cells were stained with crystal violet and colonies were counted.

### Compound Treatment of Mouse Astrocytes and Cancer Cell

Astrocytes were treated with 350 nM 4-hydroxytamoxifen or ethanol for 4 days and then cultured in DMEM/F12 medium supplemented with N-2, B-27, EGF and FGF-2 for additional 4 days. Cells isolated from tumors occurring in Lztr1^{fl/fl};Cdkn2a^{fl/fl};GFAP-Cre+ mice were cultured in medium as described above. FGFR3-TACC3 gene fusion harboring cells were similarly cultured. Cells were plated at 4,000 cells/well in 130 μl of medium in opaque white 96-well plates and after 24 hrs cells treated in six replicates with 3-fold serial dilutions of osimertinib or afatinib and bemcentinib drug combinations as indicated in FIG. 7; FIG. 15H and I for 96 hrs. Viability was determined using CellTiterGlo assay reagent (Promega, G7570) and GloMax-Multi+Microplate Multimode Reader (Promega). Experiments were repeated three times with similar results. The Bliss score was obtained by using SynergyFinder (synergyfinder.fimm.fi) (87).

### Gene Expression Analysis of Schwannoma

Gene expression RNA seq data of 24 schwannomatosis-related schwannoma and their genetic annotations were retrieved from a published study (56). RNA seq raw data were mapped to human reference (hg19) using STAR (88), and featureCounts (89) was used for transcript level quantification. Differential gene expression analysis was performed to compare LZTR1 germline mutant (n=10) and wild type (n=14) samples, using EDA seq R package (90). Functional enrichment was analyzed by Mann-Whitney-Wilcoxon gene set test (MWW-GST) (57) to identify significantly overrepresented pathways in the LZTR1 mutant subset compared to the wild type, and to compute the Normalized Enrichment Score (NES) in each single sample (GST-MWW NES>0.58 and FDR<0.05).

### Statistics

Results in graphs are expressed as means±s.d. as indicated in figure legends, for the indicated number of observations. Statistical significance was determined by the Student's t-test (two-tailed, unequal variance) using GraphPad Prism 8.0 software package (GraphPad Inc. RRID:SCR_002798) or statistical functions in Excel. p-value <0.05 is considered significant and is indicated in figure legends.

### REFERENCES

1. Frattini V, Trifonov V, Chan J M, Castano A, Lia M, A bate F, et al. The integrated landscape of driver genomic alterations in glioblastoma. Nat Genet 2013; 45(10):1141-9 doi 10.1038/ng.2734.
2. Zhang H, Cao X, Wang J, Li Q, Zhao Y, Jin X. LZTR1: A promising adaptor of the CUL3 family. Oncol Lett 2021; 22(1):564 doi 10.3892/ol.2021.12825.
3. Inoue D, Polaski J T, Taylor J, Castel P, Chen S, Kobayashi S, et al. Minor intron retention drives clonal hematopoietic disorders and diverse cancer predisposition. Nat Genet 2021; 53(5):707-18 doi 10.1038/s41588-021-00828-9.
4. Piotrowski A, Xie J, Liu Y F, Poplawski A B, Gomes A R, Madanecki P, et al. Germline loss-of-function mutations in LZTR1 predispose to an inherited disorder of multiple schwannomas. Nat Genet 2014; 46(2):182-7 doi 10.1038/ng.2855.
5. Yamamoto G L, Aguena M, Gos M, Hung C, Pilch J, Fahiminiya S, et al. Rare variants in SOS2 and LZTR1 are associated with Noonan syndrome. J Med Genet 2015; 52(6):413-21 doi 10.1136/jmedgenet-2015-103018.
6. Cancer Genome Atlas Research Network. Electronic address wbe, Cancer Genome Atlas Research N. Comprehensive and Integrative Genomic Characterization of Hepatocellular Carcinoma. Cell 2017; 169(7):1327-41 e23 doi 10.1016/j.cell.2017.05.046.
7. Allanson B M, Weber M A, Jackett L A, Chan C, Lau L, Ziegler D S, et al. Oral malignant gastrointestinal neuroectodermal tumour with junctional component mimicking mucosal melanoma. Pathology 2018; 50(6):648-53 doi 10.1016/j.pathol.2018.07.002.
8. Grobner S N, Worst B C, Weischenfeldt J, Buchhalter I, Kleinheinz K, Rudneva V A, et al. The landscape of genomic alterations across childhood cancers. Nature 2018; 555(7696):321-7 doi 10.1038/nature25480.
9. Oiso N, Sakai K, Narita T, Yanagihara S, Nishio K, Kawada A. Lymph node metastatic melanoma from ungual melanoma: Identification of somatic mutations in KIT and LZTR1. J Dermatol 2018; 45(1):e5-e6 doi 10.1111/1346-8138.14071.
10. Bianchessi D, Ibba M C, Saletti V, Blasa S, Langella T, Paterra R, et al. Simultaneous Detection of NF1, SPRED1, LZTR1, and NF2 Gene Mutations by Targeted NGS in an Italian Cohort of Suspected NF1 Patients. Genes (Basel) 2020; 11(6)doi 10.3390/genes11060671.
11. Egan C, Nicolae A, Lack J, Chung H J, Skarshaug S, Pham T A, et al. Genomic profiling of primary histiocyticsarcoma reveals two molecular subgroups Haematologica 2020; 105(4):951-60 doi 10.3324/haematol.2019.230375.
12. Lu J, Chen L. Molecular Profile of a Pituitary Rhabdomyosarcoma Arising From a Pituitary Macroadenoma: A Case Report. Front Endocrinol (Lausanne) 2021; 12:752361 doi 10.3389/fendo.2021.752361.
13. Cohen-Barak E, Toledano-Alhadef H, Danial-Farran N, Livneh I, Mwassi B, Hriesh M, et al. Concomitant variants in NF1, LZTR1 and GNAZ genes probably contribute to the aggressiveness of plexiform neurofibroma and warrant treatment with MEK inhibitor. Exp Dermatol 2022; 31(5):775-80 doi 10.1111/exd.14514.
14. Zhang W, Wang T, Wang Y, Zhu F, Shi H, Zhang J, et al. Intratumor heterogeneity and clonal evolution revealed in castration-resistant prostate cancer by longitudinal genomic analysis. Transl Oncol 2022; 16:101311 doi 10.1016/j.tranon.2021.101311.
15. Banerjee S, Zhang X, Kuang S, Wang J, Li L, Fan G, et al. Comparative analysis of clonal evolution among patients with right- and left-sided colon and rectal cancer. iScience 2021; 24(7):102718 doi 10.1016/j.isci.2021.102718.
16. Gao J, Aksoy B A, Dogrusoz U, Dresdner G, Gross B, Sumer S O, et al. Integrative analysis of complex cancer genomics and clinical profiles using the cBioPortal. Sci Signal 2013; 6(269):pl1 doi 10.1126/scisignal.2004088.
17. Evans D G, Bowers N L, Tobi S, Hartley C, Wallace A J, King A T, et al. Schwannomatosis: a genetic and epidemiological study. J Neurol Neurosurg Psychiatry 2018; 89(11):1215-9 doi 10.1136/jnnp-2018-318538.
18. Bigenzahn J W, Collu G M, Kartnig F, Pieraks M, Vladimer G I, Heinz L X, et al. LZTR1 is a regulator of RAS ubiquitination and signaling. Science 2018; 362(6419):1171-7 doi 10.1126/science.aap8210.
19. Steklov M, Pandolfi S, Baietti M F, Batiuk A, Carai P, Najm P, et al. Mutations in LZTR1 drive human disease by dysregulating RAS ubiquitination. Science 2018; 362(6419):1177-82 doi 10.1126/science.aap7607.
20. Castel P, Cheng A, Cuevas-Navarro A, Everman D B, Papageorge A G, Simanshu D K, et al. RIT1 oncoproteins escape LZTR1-mediated proteolysis. Science 2019; 363(6432):1226-30 doi 10.1126/science.aav1444.
21. Meyer A S, Miller M A, Gertler F B, Lauffenburger D A. The receptor AXL diversifies EGFR signaling and limits the response to EGFR-targeted inhibitors in triple-negative breast cancer cells. Sci Signal 2013; 6(287):ra66 doi 10.1126/scisignal.2004155.
22. Levkowitz G, Waterman H, Ettenberg S A, Katz M, Tsygankov A Y, Alroy I, et al. Ubiquitin ligase activity and tyrosine phosphorylation underlie suppression of growth factor signaling by c-Cbl/Sli-1. Mol Cell 1999; 4(6):1029-40 doi 10.1016/s1097-2765(00)80231-2.
23. Sorkin A, Goh L K. Endocytosis and intracellular trafficking of ErbBs. Exp Cell Res 2008; 314(17):3093-106 doi 10.1016/j.yexcr.2008.08.013.
24. Sorkin A, Von Zastrow M. Signal transduction and endocytosis: close encounters of many kinds. Nat Rev Mol Cell Biol 2002; 3(8):600-14 doi 10.1038/nrm883.
25. Soucy T A, Smith P G, Milhollen M A, Berger A J, Gavin J M, Adhikari S, et al. An inhibitor of NEDD8-activating enzyme as a new approach to treat cancer. Nature 2009; 458(7239):732-6 doi 10.1038/nature07884.
26. Huang F, Zeng X, Kim W, Balasubramani M, Fortian A, Gygi S P, et al. Lysine 63-linked polyubiquitination is required for EGF receptor degradation. Proc Natl Acad Sci USA 2013; 110(39):15722-7 doi 10.1073/pnas.1308014110.
27. Smith M J, Isidor B, Beetz C, Williams S G, Bhaskar S S, Richer W, et al. Mutations in LZTR1 add to the complex heterogeneity of schwannomatosis. Neurology 2015; 84(2):141-7 doi 10.1212/WNL.0000000000001129.
28. Tate J G, Bamford S, Jubb H C, Sondka Z, Beare D M, Bindal N, et al. COSMIC: the Catalogue Of Somatic Mutations In Cancer. Nucleic Acids Res 2019; 47(D1):D941-D7 doi 10.1093/nar/gkyl015.
29. Haglund K, Dikic I. The role of ubiquitylation in receptor endocytosis and endosomal sorting. J Cell Sci 2012; 125(Pt 2):265-75 doi 10.1242/jcs.091280.
30. Lemmon M A, Schlessinger J. Cell signaling by receptor tyrosine kinases. Cell 2010; 141(7):1117-34 doi 10.1016/j.cell.2010.06.011.
31. Schlessinger J. Cell signaling by receptor tyrosine kinases. Cell 2000; 103(2):211-25 doi 10.1016/s0092-8674(00)00114-8.
32. Freed D M, Bessman N J, Kiyatkin A, Salazar-Cavazos E, Byrne P O, Moore J O, et al. EGFR Ligands Differentially Stabilize Receptor Dimers to Specify Signaling Kinetics. Cell 2017; 171(3):683-95 e18 doi 10.1016/j.cell.2017.09.017.
33. Huang F, Kirkpatrick D, Jiang X, Gygi S, Sorkin A. Differential regulation of EGF receptor internalization and degradation by multiubiquitination within the kinase domain. Mol Cell 2006; 21(6):737-48 doi 10.1016/j.molcel.2006.02.018.
34. Eden E R, Huang F, Sorkin A, Futter C E. The role of EGF receptor ubiquitination in regulating its intracellular traffic. Traffic 2012; 13(2):329-37 doi 10.1111/j.1600-0854.2011.01305.x.
35. Huang F, Goh L K, Sorkin A. EGF receptor ubiquitination is not necessary for its internalization. Proc Natl Acad Sci USA 2007; 104(43):16904 doi 10.1073/pnas.0707416104.
36. Skarnes W C, Rosen B, West A P, Koutsourakis M, Bushell W, Iyer V, et al. A conditional knockout resource for the genome-wide study of mouse gene function. Nature 2011; 474(7351):337-42 doi 10.1038/nature10163.
37. Ogawa R, Fujita K, Ito K. Mouse embryonic dorsal root ganglia contain pluripotent stem cells that show features similar to embryonic stem cells and induced pluripotent stem cells. Biol Open 2017; 6(5):602-18 doi 10.1242/bio.021758.
38. Lawson S N, Biscoe T J. Development of mouse dorsal root ganglia: an autoradiographic and quantitative study. J Neurocytol 1979; 8(3):265-74 doi 10.1007/BF01236122.
39. Jessen K R, Mirsky R. Schwann Cell Precursors; Multipotent Glial Cells in Embryonic Nerves. Front Mol Neurosci 2019; 12:69 doi 10.3389/fnmol.2019.00069.
40. Le L Q, Shipman T, Burns D K, Parada L F. Cell of origin and microenvironment contribution for NF1-associated dermal neurofibromas. Cell Stem Cell 2009; 4(5):453-63 doi 10.1016/j.stem.2009.03.017.
41. Toma J G, Akhavan M, Fernandes K J, Barnabe-Heider F, Sadikot A, Kaplan D R, et al. Isolation of multipotent adult stem cells from the dermis of mammalian skin. Nat Cell Biol 2001; 3(9):778-84 doi 10.1038/ncb0901-778.
42. Zhu Y, Guignard F, Zhao D, Liu L, Burns D K, Mason R P, et al. Early inactivation of p53 tumor suppressor gene cooperating with NF1 loss induces malignant astrocytoma. Cancer Cell 2005; 8(2):119-30 doi 10.1016/j.ccr.2005.07.004.
43. Meuwissen R, Linn S C, Linnoila R I, Zevenhoven J, Mooi W J, Berns A. Induction of small cell lung cancer by somatic inactivation of both Trp53 and Rb1 in a conditional mouse model. Cancer Cell 2003; 4(3):181-9 doi 10.1016/s1535-6108(03)00220-4.
44. Marino S, Vooijs M, van Der Gulden H, Jonkers J, Berns A. Induction of medulloblastomas in p53-null mutant mice by somatic inactivation of Rb in the external granular layer cells of the cerebellum. Genes Dev 2000; 14(8):994-1004.
45. Chan S H, Chiang J, Ngeow J. CDKN2A germline alterations and the relevance of genotype-phenotype associations in cancer predisposition. Hered Cancer Clin Pract 2021; 19(1):21 doi 10.1186/s13053-021-00178-x.
46. Sargen M R, Merrill S L, Chu E Y, Nathanson K L. CDKN2A mutations with p14 loss predisposing to multiple nerve sheath tumours, melanoma, dysplastic naevi and internal malignancies: a case series and review of the literature. Br J Dermatol 2016; 175(4):785-9 doi 10.1111/bjd.14485.
47. Chaney K E, Perrino M R, Kershner L J, Patel A V, Wu J, Choi K, et al. Cdkn2a Loss in a Model of Neurofibroma Demonstrates Stepwise Tumor Progression to A typical Neurofibroma and MPNST. Cancer Res 2020; 80(21):4720-30 doi 10.1158/0008-5472.CAN-19-1429.
48. Pemov A, Hansen N F, Sindiri S, Patidar R, Higham C S, Dombi E, et al. Low mutation burden and frequent loss of CDKN2A/B and SMARCA2, but not PRC2, define premalignant neurofibromatosis type 1-associated atypical neurofibromas. Neuro Oncol 2019; 21(8):981-92 doi 10.1093/neuonc/noz028.
49. Brohl A S, Kahen E, Yoder S J, Teer J K, Reed D R. The genomic landscape of malignant peripheral nerve sheath tumors: diverse drivers of Ras pathway activation. Sci Rep 2017; 7(1):14992 doi 10.1038/s41598-017-15183-1.
50. Nielsen G P, Stemmer-Rachamimov A O, Ino Y, Moller M B, Rosenberg A E, Louis DN. Malignant transformation of neurofibromas in neurofibromatosis 1 is associated with CDKN2A/p16 inactivation. Am J Pathol 1999; 155(6):1879-84 doi 10.1016/S0002-9440(10)65507-1.
51. Fine S W, McClain S A, Li M. Immunohistochemical staining for calretinin is useful for differentiating schwannomas from neurofibromas. Am J Clin Pathol 2004; 122(4):552-9 doi 10.1309/AGBG-TBRJ-4W0B-C7LN.
52. Rodriguez F J, Folpe A L, Giannini C, Perry A. Pathology of peripheral nerve sheath tumors: diagnostic overview and update on selected diagnostic problems. Acta Neuropathol 2012; 123(3):295-319 doi 10.1007/s00401-012-0954-z.
53. Sasaki M, Knobbe C B, Itsumi M, Elia A J, Harris I S, Chio, I I, et al. D-2-hydroxyglutarate produced by mutant IDH1 perturbs collagen maturation and basement membrane function. Genes Dev 2012; 26(18):2038-49 doi 10.1101/gad.198200.112.
54. Johnston J J, van der Smagt J J, Rosenfeld J A, Pagnamenta A T, Alswaid A, Baker E H, et al. Autosomal recessive Noonan syndrome associated with biallelic LZTR1 variants. Genet Med 2018; 20(10):1175-85 doi 10.1038/gim.2017.249.
55. Fuhrich D G, Lessey B A, Savaris R F. Comparison of HSCORE assessment of endometrial beta3 integrin subunit expression with digital HSCORE using computerized image analysis (ImageJ). Anal Quant Cytopathol Histpathol 2013; 35(4):210-6.
56. Mansouri S, Suppiah S, Mamatjan Y, Paganini I, Liu J C, Karimi S, et al. Epigenomic, genomic, and transcriptomic landscape of schwannomatosis. Acta Neuropathol 2021; 141(1):101-16 doi 10.1007/s00401-020-02230-x.
57. Frattini V, Pagnotta S M, Tala, Fan J J, Russo M V, Lee S B, et al. A metabolic function of FGFR3-TACC3 gene fusionsin cancer. Nature 2018; 553(7687):222-7 doi 10.1038/nature25171.
58. Holland S J, Pan A, Franci C, Hu Y, Chang B, Li W, et al. R428, a selective small molecule inhibitor of Axl kinase, blocks tumor spread and prolongs survival in models of metastatic breast cancer. Cancer Res 2010; 70(4):1544-54 doi 10.1158/0008-5472.CAN-09-2997.
59. Solca F, Dahl G, Zoephel A, Bader G, Sanderson M, Klein C, et al. Target binding properties and cellular activity of afatinib (BIBW 2992), an irreversible ErbB family blocker. J Pharmacol Exp Ther 2012; 343(2):342-50 doi 10.1124/jpet.112.197756.
60. Cross D A, Ashton S E, Ghiorghiu S, Eberlein C, Nebhan C A, Spitzler P J, et al. AZD9291, an irreversible EGFR TKI, overcomes T790M-mediated resistance to EGFR inhibitors in lung cancer. Cancer Discov 2014; 4(9):1046-61 doi 10.1158/2159-8290.CD-14-0337.
61. Han L, Zhang X, Wang Z, Zhang X, Zhao L, Fu W, et al. SH-1028, An Irreversible Third-Generation EGFR TKI, Overcomes T790M-Mediated Resistance in Non-Small Cell Lung Cancer. Front Pharmacol 2021; 12:665253 doi 10.3389/fphar.2021.665253.
62. Liu X, Chen X, Shi L, Shan Q, Cao Q, Yue C, et al. The third-generation EGFR inhibitor AZD9291 overcomes primary resistance by continuously blocking ERK signaling in glioblastoma. J Exp Clin Cancer Res 2019; 38(1):219 doi 10.1186/s13046-019-1235-7.
63. Ruan Z, Kannan N. Altered conformational landscape and dimerization dependency underpins the activation of EGFR by alphaC-beta4 loop insertion mutations. Proc Natl Acad Sci USA 2018; 115(35):E8162-E71 doi 10.1073/pnas.1803152115.
64. Floc'h N, Martin M J, Riess J W, Orme J P, Staniszewska A D, Menard L, et al. Antitumor Activity of Osimertinib, an Irreversible Mutant-Selective EGFR Tyrosine Kinase Inhibitor, in NSCLC Harboring EGFR Exon 20 Insertions. Mol Cancer Ther 2018; 17(5):885-96 doi10.1158/1535-7163.MCT-17-0758.
65. Singh D, Chan J M, Zoppoli P, Niola F, Sullivan R, Castano A, et al. Transforming fusions of FGFR and TACC genes in human glioblastoma. Science 2012; 337(6099):1231-5 doi 10.1126/science.1220834.
66. Stogios P J, Downs G S, Jauhal J J, Nandra S K, Prive G G. Sequence and structural analysis of BTB domain proteins. Genome Biol 2005; 6(10):R82 doi 10.1186/gb-2005-6-10-r82.
67. Clandinin T R, DeModena J A, Sternberg P W. Inositol trisphosphate mediates a RAS-independent response to LET-23 receptor tyrosine kinase activation in C. elegans. Cell 1998; 92(4):523-33 doi 10.1016/s0092-8674(00)80945-9.
68. Mishra S, Smolik S M, Forte M A, Stork P J. Ras-independent activation of ERK signaling via the torso receptor tyrosine kinase is mediated by Rap1. Curr Biol 2005; 15(4):366-70 doi 10.1016/j.cub.2005.02.022.
69. Ammoun S, Cunliffe C H, Allen J C, Chiriboga L, Giancotti F G, Zagzag D, et al. ErbB/HER receptor activation and preclinical efficacy of lapatinib in vestibular schwannoma. Neuro Oncol 2010; 12(8):834-43 doi 10.1093/neuonc/noq012.
70. Hutterer M, Knyazev P, Abate A, Reschke M, Maier H, Stefanova N, et al. Axl and growth arrest-specific gene 6 are frequently overexpressed in human gliomas and predict poor prognosis in patients with glioblastoma multiforme. Clin Cancer Res 2008; 14(1):130-8 doi 10.1158/1078-0432.CCR-07-0862.
71. Munoz-Hidalgo L, San-Miguel T, Megias J, Monleon D, Navarro L, Roldan P, et al. Somatic copy number alterations are associated with EGFR amplification and shortened survival in patients with primary glioblastoma. Neoplasia 2020; 22(1):10-21 doi 10.1016/j.neo.2019.09.001.
72. Di Fiore P P, Pierce J H, Fleming T P, Hazan R, Ullrich A, King C R, et al. Overexpression of the human EGF receptor confers an EGF-dependent transformed phenotype to NIH 3T3 cells. Cell 1987; 51(6):1063-70 doi 10.1016/0092-8674(87)90592-7.
73. Hu C, Leche C A, 2nd, Kiyatkin A, Y u Z, Stayrook S E, Ferguson K M, et al. Glioblastoma mutations alter EGFR dimer structure to prevent ligand bias. Nature 2022; 602(7897):518-22 doi 10.1038/s41586-021-04393-3.
74. Vermi W, Giurisato E, Lonardi S, Balzarini P, Rossi E, Medicina D, et al. Ligand-dependent activation of EGFR in follicular dendritic cells sarcoma is sustained by local production of cognate ligands. Clin Cancer Res 2013; 19(18):5027-38 doi 10.1158/1078-0432.CCR-13-1275.
75. Sigismund S, Avanzato D, Lanzetti L. Emerging functions of the EGFR in cancer. Mol Oncol 2018; 12(1):3-20 doi 10.1002/1878-0261.12155.
76. Zhu C, Wei Y, Wei X. AXL receptor tyrosine kinase as a promising anti-cancer approach: functions, molecular mechanisms and clinical applications. Mol Cancer 2019; 18(1):153 doi 10.1186/s12943-019-1090-3.
77. Durkin M E, Qian X, Popescu N C, Lowy D R. Isolation of Mouse Embryo Fibroblasts. Bio Protoc 2013; 3(18) doi 10.21769/bioprotoc.908.
78. HeY, Taylor N, Bhattacharya A. Isolation and Culture of Astrocytes from Postnatal and Adult Mouse Brains. Methods Mol Biol 2019; 1938:37-47 doi 10.1007/978-1-4939-9068-9_3.
79. Garofano L, Migliozzi S, Oh Y T, D'Angelo F, Najac R D, Ko A, et al. Pathway-based classification of glioblastoma uncovers a mitochondrial subtype with therapeutic vulnerabilities. Nature Cancer 2021; 2(2):141-56 doi 10.1038/s43018-020-00159-4.
80. Cox J, Mann M. MaxQuant enables high peptide identification rates, individualized p.p.b.-range mass accuracies and proteome-wide protein quantification. Nature biotechnology 2008; 26(12):1367-72 doi 10.1038/nbt.1511.
81. Cox J, Neuhauser N, Michalski A, Scheltema R A, Olsen J V, Mann M. Andromeda: a peptide search engine integrated into the MaxQuant environment. J Proteome Res 2011; 10(4):1794-805 doi 10.1021/pr101065j.
82. Navarrete-Perea J, Y u Q, Gygi S P, Paulo J A. Streamlined Tandem Mass Tag (SL-TMT) Protocol: An Efficient Strategy for Quantitative (Phospho) proteome Profiling Using Tandem Mass Tag-Synchronous Precursor Selection-MS3. J Proteome Res 2018; 17(6):2226-36 doi 10.1021/acs.jproteome.8b00217.
83. Shevchenko A, Tomas H, Havlis J, Olsen J V, Mann M. In-gel digestion for mass spectrometric characterization of proteins and proteomes. Nature protocols 2006; 1(6):2856-60 doi 10.1038/nprot.2006.468.
84. Lee S B, Frattini V, Bansal M, Castano A M, Sherman D, Hutchinson K, et al. An ID2-dependent mechanism for VHL inactivation in cancer. Nature 2016; 529(7585):172-7 doi10.1038/nature16475.
85. Niola F, Zhao X, Singh D, Sullivan R, Castano A, Verrico A, et al. Mesenchymal high-grade glioma is maintained by the ID-RAP1 axis. J Clin Invest 2013; 123(1):405-17 doi 10.1172/J C163811.
86. Bradley A, Anastassiadis K, Ayadi A, Battey J F, Bell C, Birling M C, et al. The mammalian gene function resource: the International Knockout Mouse Consortium. Mamm Genome 2012; 23(9-10):580-6 doi 10.1007/s00335-012-9422-2.
87. Ianevski A, Giri A K, Aittokallio T. SynergyFinder 2.0: visual analytics of multi-drug combination synergies. Nucleic Acids Res 2020; 48(W1):W488-W93 doi 10.1093/nar/gkaa216.
88. Dobin A, Davis C A, Schlesinger F, Drenkow J, Zaleski C, J ha S, et al. STAR: ultrafast universal RNA-seq aligner. Bioinformatics 2013; 29(1):15-21 doi 10.1093/bioinformatics/bts635.
89. Liao Y, Smyth G K, Shi W. featureCounts: an efficient general purpose program for assigning sequence reads to genomic features. Bioinformatics 2014; 30(7):923-30 doi 10.1093/bioinformatics/btt656.
90. Risso D, Schwartz K, Sherlock G, Dudoit S. GC-content normalization for RNA-Seq data. BMC Bioinformatics 2011; 12:480 doi 10.1186/1471-2105-12-480.

List 1. List of ubiquitylated peptides from DiGly proteomics assay: LZTR1, PKM, RSU1, RPL28, SND1, KCTD10, MYL6, SH3GLB1, RBBP7, UCK1, PRKCDBP, EHD1, CCT2, AKR1B8, WSB1, IKBKB, DVL2, PSMC5, MACF1, POLR2B, ATP1B3, NONO, NQO1, FAM129B, RPL4, APLP2, ATRX, WLS, LRP1, LRRFIP1, RFWD3, DDX3Y, RPL18A, EEF2, FASN, LAMA2, PRPF8, ACTN4, LARS, NISCH, TBC1D15, PGD, RAN, RBMS2, LTN1, BCAR1, SAT1, FIG4, TCAF1, GCAT, DUSP4, POLA2, CLASP2, PSMD14, TGFB1, SUMO3, SRP54, TPP2, DYNC1H1, UBR4, AKT1, NANS, PRIM1, MYO1C, KLHL11, C1GALT1C1, DDB1, ITPR1, TOMM70A, FLNB, RASA3, NCBP2, CTNND1, MYOF, VPS37B, IGF2BP3, CC2D1A, DPY19L3, MAP4K1, CDH2, CTH, PSMC4, HCFC1, ALG11, ITPR3, SERPINH1, HDGFRP2, MED14, AHSA2, MVP, UBTF, MID1IP1, VARS, PNP, DPP9, DPYSL2, ACLY, VDAC1, IMPDH2, WDR45, ABCD3, XRN1, POLR1D, DIAPH3, PMAIP1, CEP350, RICTOR, ZDHHC20, APPBP2, GRIN2A, NDUFS1, FOCAD, PGK1, NBR1, PITRM1, HSPA1B, ERP44, CCT5, CTSD, TFIP11, CCT4, OSBPL3, JAG1, HMGCS1, RPLP0, DYNLL1, ATP6V1H, IDE, PPP1R13L, CAPN2, ENO1, FUNDC2, OSR1, LOX, ATG101, NUDT5, INTS7, UBA2, SEC23A, JCAD, ITGB5, TRAPPC13, UBE4A, FLNA, LYAR, KDM5A, KLHL7, IARS, PLEC, SPICE1, FERMT2, ANXA5, ALG13, IF135, EIF3A, USP40, SREBF1, EIF2A, SPRTN, TRIM41, NDUFA13, KIAA1524, ARL2, PSMD3, HSPD1, TBCE, 44257, CMTR1, ANGPTL4, SLC4A2, CCT8, YY1, BFAR, RABGEF1, PFAS, ARHGAP10, UBR5, IMPDH1, PXDN, CSPG4, RGS16, FECH, AXL, TNS3, TRDMT1, POLR2A, SNX5, PSMB7, ATP6A P2, CYB5R1, CTR9, GMPPB, LRRC8C, SMARCD2, STAM, FANCD2, LTBP3, IQSEC1, CUL7, EZH2, CPSF1, BSG, VPS13A, GTF3C1, ZZEF1, ARPC1B, TTC3, HSP90AA 1, USP7, AFAP1, PREPL, PFN1, NPR3, EEF1G, OTOF, PHLDB1, ARFGAP3, RPS29, TNKS2, NMT1, CYFIP1, ACTN1, NEDD4, PRRX1, GNPNAT1, AP3B1, XRCC5, NRDE2, NFKB2, ATP6V1C1, HNRNPU, HNRNPM, GPR56, PIAS4, SBF2, VPS26A, MAGOHB, BRE, EIF5A, SCAF1, KLHL23, PLEKHH2, AATF, OBSL1, MTA1, RSRP1, CHD8, DIP2B, HARS, GPAM, HERC2, HIP1, NUFIP2, TMEM159, TECR, ERN1, PDE8A, BASP1, RCBTB2, HMGCR, ENC1, ABCD1, CAPN7, UHRF2, TMEM39B, PPME1, POLQ, SNRPB2, UBR1, PLCB3, LAMB2, FNBP1L, SNAPC2, PLXNB2, RPL7A, RSBN1, VIM, TMEM45A, DAPK1, PGAM5, IAH1, SH3PXD2A, NCOR1, PIK3C3, PDXDC1, HAUS5, RPL36, SPG11, USP48, HSPG2, USP13, OCRL, ZMYND11, CENPH, AEBP1, JMJD1C, SHMT2, SNAPC3, GTF2F1, NACC2, LRP6, LMNB1, VAMP7, OSBP, PBRM1, GHR, SVIL, MRPL46, LAS1L, TRRAP, NT5C3B, WDR48, SUPT16H, STXBP4, TBL3, ANGEL2, LUZP1, BPNT1, ALG2, KDM1A, PYGB, SCAMP1, RPA1, ELMO2, RPS19, ADSL, MRPL24, PIKFYVE, GNAO1, EPHA2, RHOBTB3, AP2M1, CAMK2G, LRRCC1, TSEN2, CDKAL1, TMC01, ECI2, KNTC1, LRRC40, FAM120A, SGK3, JUNB, ERGIC1, CEBPZ, GLE1, SKI, TRIM62, ABCC1, CAPZA2, PIGX, MGAT1, WDR91, ABCA7, RABAC1, DENND4C, SWT1, ATAD1, ECH1, POLR3B, RP1, REV1, SETD8, CHD6, MIB2, DNPEP, FLOT1, TPR, RANBP2, MLX, RARS, CAD, COA3, NAMPT, ARL4D, DPY19L4, SLC7A6, TOP1, DNAJA3, OPA1, ERI3, DSTYK, PPP4R1, LIG1, TAX1BP1, UBE3C, SPTBN1, USP33, GNAQ, KIFC1, SENP5, COMMD1, HSD17B7, METTL3, VPS35, SESN3, TCTN1, ST7L, ADAMTS2, ACTR3, RRS1, CHTF18, ESYT2, NCL, SMARCE1, ID2, SIN3B, STAT3, HEBP1, CDC5L, MTA3, CENPC, MAPK14, ANAPC16, TBC1D8B, TAF1A, CCAR2, XRN2, CAV1, HPS5, TENM3, JUP, USP47, RPL11, EHD2, NRIP1, NFRKB, MICAL2, CNOT2, ALDH3B1, COL5A1, HJURP, KDM3A, CDK1, MCM4, H2-D1, RPS11, VPS18, MCL1, NOP56, MTAP, MIIP, TPD52L2, CDC73, KIAA1429, RPL26, CDC45, CKS2, PTGIS, KARS, MTCH2, THOP1, RNF6, GANAB, PPP2R2D, ACP2, RPL14, ESF1, OAT, GRB10, CTNNA1, PITPNA, ECM29, TBL2, SNX9, VPS33A, SLC25A4, FAM73B, 44449, TNFAIP1, SUPT6H, DLD, RPL37A, DCAKD, SCO2, PSMD2, PPP1CA, DDX1, NUDCD3, NUBP2, USP24, TIMM17B, ANP32E, ARCN1, RPL21, SCD2, CPNE1, PHLDA1, ACOT7, PDE12, FZD7, ASPSCR1, VAT1, EIF3B, ITGAV, LSG1, SNX2, HSPH1, USP5, IGBP1, EIF253X, SLC25A5, TKT, MFN2, CHAF1B, MEF2BNB, DHX9, HADHA, CLDND1, SDC2, MYO10, PSMC2, CMIP, RIN1, SOAT1, CWF19L2, PAF1, RPS15A, YEATS2, ARPC2, MTMR6, MARF1, TBCB, IGF2BP2, ADPRH, DNM2, UGDH, KANK2, GTF21, CIRH1A, DDX5, METAP2, GLUD1, HSP90AB1, ANXA6, GATAD2A, TDG, DNAJC5, CLIC1, SEC63, TUBGCP3, TSPYL2, ORC4, CHUK, IFT74, CDC25C, STK25, PBDC1, ARMCX3, TOP2A, FAR1, EMC8, UAP1L1, DCTN2, RPS4X, TRIM28, RPS18, NCAPD2, ASB13, KDM3B, ERLEC1, PRELID1, SKA2, PTGS2, ATP5A1, DCAF12, TBC1D31, FAM91A1, DPY19L1, HIF1A, BTG3, FBL, RPS5, SNRPB, ANKRD1, DYSF, WDFY3, PDHA1, ADSS, JAK1, PCNXL3, YBX3, S100A4, COX4I1, RRM1, DPH2, TRPC4AP, MYH10, LRRK2, WDR74, KLHL21, PELO, AP1M1, SMYD2, SERINC1, TMOD3, EIF2B5, SLC25A3, CDK11B, HSPA13, PPP4C, RPS3, ECI1, RAB3GAP2, PHGDH, ACKR3, PPM1G, CD109, RFWD2, DTX3L, LRP2, MED10, MAGED1, TOLLIP, MRPL20, OSBPL5, ILDR1, NCAPD3, GNPTG, FTSJ3, DNMBP, SERPINB1A, OXCT1, 44448, DCAF7, RASSF3, PHB2, RPL7, TRIP4, CENPP, UBIAD1, SCARB1, RUVBL1, NPM1, APPL1, SH3GLB2, RPL19, SUMO2, HMG20B, MAOA, COL6A2, AHCTF1, NGDN, TARS, STAMBP, FADS2, SARS, FAM65A, FAM45A, RRAGC, PML, UBXN6, MCM2, ATP6V1A, CAPZA1, SNRPD2, YTHDF2, NCBP1, POLE, ZWINT, CCT3, PLEKHF1, EIF2B2, BIN3, MCM5, GHITM, NATD1, CRLF1, VPS36, MAPK3, ZCCHC24, WDR60, SEH1L, ABCE1, NSUN4, SFXN3, BZW1, TCP1, POMP, TUBGCP2, CELF1, SIK1, VCPIP1, PSMD6, CMAS, BIRC2, AACS, USE1, PGP, NTAN1, DOCK7, GON4L, SDAD1, GGA2, AKAP1, SAMD9L, NUP93, MSN, ANAPC4, DOCK5, UBE20, HMOX1, ABCF3, KATNA1, N4BP2L2, ASCC3, CIAPIN1, IER3IP1, RPN2, STK38L, RENBP, ZNFX1, CHP1, EEF1A1, BUB3, BCL2L13, POLK, POLR2C, POLH, MON1A, VCP, RALY, GLIPR2, ARHGEF1, TAGLN, XBP1, STAT6, GTF2H4, ABCB1B, MTHFD1L, MAPK1, COX6B1, DIS3L, SCFD1, RANBP1, GDI2, NFE2L2, CHKA, COPZ1, POLE2, ITSN2, ALDOA, AIMP1, VPS33B, ATP1A1, GOPC, PSMA5, SKAP2, EIF3C, ARID5A, HIBADH, AARS, GSK3A, RPL10, EPRS, TIGD5, FPGS, CCNE1, TXNDC5, PPIL2, UBE3A, PLSCR1, SLC25A25, SMS, ACSL4, SPEN, IRF2BP1, SENP3, MTPAP, GNL3L, ALDH3A2, SMG8, ACAT1, LXN, NDUFB10, RPS17, MDH2, MRE11A, NUDT16L1, EIF251, ANXA3, RNH1, GGCT, RPS25, STK38, TUBB3, IDH1, PDCD11, TARDBP, KPNA4, NUP107, GPX8, SEC31A, YPEL5, PAN3, PGAM1, PHKA2, TMED3, ANXA2, TOB2, BTG1, RPS24, TOPORS, OCIAD1, IFRD1, HNRNPAB, SIL1, ATP2C1, CERS5, SNX18, FBXO42, TSTD2, ALAS1, MPC2, CSTF1, PCNA, ARF1, IDI1, MGA, MICU1, TXNRD1, NCSTN, UPF1, GAS8, TSC2, CD80, ZXDB, NUP133, FBXO30, LARP4, PCM1, USP22, BMPR1A, GK, BGN, XPO5, RPS23, MCM6, MTFR1L, ELAVL1, ARHGEF40, SPC25, PSME4, G6PDX, MAP2K3, BAZ1A, DDX6, FAF2, POLR1C, GYS1, IPO5, SNAP29, PLK2, NUDCD2, MAPKAPK2, MTX2, PSMC6, NACC1, FN1, DOCK6, UBE2N, NEU3, TOMM20, RGS20, FIBP, PROSC, MYO7A, PRKACA, ARHGDIA, NOP58, PYCR1, RUFY2, ASNS, MAP2K2, DARS, PFKP, RB1CC1, INTS1, SDHAF2, CLEC12A, FBX03, HERC4, RPS9, STAT1, PA2G4, NUP88, ATP2A2, GPD2, INTS4, FAM98B, RPS13, SGCD, ILVBL, HNRNPH2, CDC25B, EFEMP1, RNF145, SLU7, ARL8B, SRBD1, SNAP23, TOP2B, TEX10, SUN1, MAD1L1, ANAPC7, MST01, TSC22D1, YWHAG, NAE1, SNX4, SEC61A1, MRPL39, TSR1, FNDC3B, USP14, GCLC, ATAD2, PAPOLA, RIT1, SNRNP200, GSTO1, PITPNM1, RAD21, LMNA, CDC16, PNO1, PPP1R7, EIF3L, MYH9, ASS1, NDUFA9, POLR1A, DNMT1, METTL6, OBFC1, CCT6A, PDIA4, ELP3, ARMCX6, MTHFD2, PREP, EXD2, CCT7, SF1, TRAP1, INTS3, HAUS1, FBLN5, DRG1, FBXL5, CAPN1, WARS, ARHGEF25, FAM83D, VCAN, DAPK3, TIMM17A, BIRC6, RPL6, NXN, ILK, CHPF, NT5DC3, NAA15, ALAD, MYL12B, VPS52, BRMS1L, DCUN1D5, ATF4, SERPINF1, NHP2L1, ACTR2, MYO5A, UQCRC1, SLC12A4, MED27, CCRN4L, NID1, SBNO2, DONSON, DHX30, MORC2A, SEC23IP, UTP20, NUP160, GNB2, CCDC93, ACACA, IPO8, CTPS1, CFAP36, PSMB2, SGPL1, SOX3, DHX15, MGEA5, TMEM189, TNFRSF12A, ANAPC10, CASP2, RAP1B, STK17B, KCNAB1, ETS2, MYO1E, EEF1B, TMX3, NUP37, AK2, NOP14, KLHDC2, NQO2, CERS2, ZBTB33, NSFL1C, PRUNE2, FLNC, CC2D2A, GRIA3, TBX15, IREB2, ATP5F1, DRC7, MICU2, HEATR5B, HSPA9, PHB, TPM3, SQLE, TNFAIP2, CD44, MRPL45, RPS6KC1, CRYAB, G3BP1, SPG20, TAF1, SEC14L1, SAMHD1, UBL7, CSNK2B, NCAPH, FOPNL, EIF3H, ECE1, HAUS7, YME1L1, DOHH, EGFR, USP25, UBE4B, WRNIP1, PCBP2, RBM14, PRPS1, SPCS2, FPGT, PRPF19, NUDT1, GTPBP4, PTCD3, PSMB5, CSNK1D, DPH6, TRIM32, XPNPEP1, FRMD8, CAMLG, CCDC80, ATXN2L, ASPM, R3HDM2, UFSP2, AGO2.

List 2. List of proteins differentially abundant in LZTR1−/− compared to LZTR1+/+ cells quantified by TMT: RIT1, DES, LY Z2, PGK2, MRAS, MYLK, GVIN1, MGP, TDPOZ4, ANKH, LCP1, TGFB1, VNN1, HOXD9, COL8A1, FST, ALDH1A7, APOA1, ILDR2, TSPAN4, ISLR, OLFR1038, SCRN3, G6PC3, PITPNM2, FZD1, NNMT, MAN2A2, BDH2, SYTL2, EGFR, CTSH, PURG.

List 3. List of top scoring LZTR1 interacting proteins from immunoprecipitation/mass spectrometry: RNF213, CDK5RAP2, AXL, POLR1A, VPS51, TUBGCP2, ARHGEF40, MTR, TRIO, YME1L1, KIAA1217, KNL1, RUSC2, VPS13A, CY P51A1, HECTD3, RICTOR, NCAPG2, BRAT1, PARP14, HERC2, PAPPA, TRAPPC12, TELO2, AMBRA1, SEC24D, BUB1, KLHL21, 44262, UBXN6, DPP9, TTI1, SMG9, EMC1, SNX14, PTPN23, SMPD4, ARFGEF1, PARP10, GBE1, TRIM47, FADS1, ELP1, PPFIBP1, RANBP6, ATF6, MTOR, USP47, KDM5C, STK11IP, AHR, MCMBP, SHKBP1, POLR3A, AMOTL2, KIF1B, ZCCHC6, CTH, MAP4K4, KIAA1468, UFL1, URB1, BRCA2, LONP2, INTS1, MYO10, VCAN, ASXL1, TRRAP, WDR6, LAMB3, RALGAPB, ANKRD52, CREB3L2, GLE1, CDC42BPB, PNPLA2, SPG11, HELZ, ARMCX2, SPRYD3, USP4, CARS, NCAPD3, THADA, RAP1GDS1, UTP6, SCYL1, GMPPA, RANBP9, RTTN, PEAK1, VPS18, UMPS, SHCBP1, APEH, RETSAT, PDLIM7, SKIV2L, RPAP1, ATG13, KLHL26, MAPRE2, PLK1, PHLDA1, SLC4A2, HTT, ELAC2, DHX32, CCNB2, ERGIC3, GUCY1B1, PTOV1, TRAPPC11, MELK, TMC04, CDC42BPA, ATAD2, RABGEF1, FASTKD2, UBE4B, ALPK2, SREBF2, CA9, EIF2AK4, ARHGAP31, NDC80, EPG5, ATF6B, PSMD5, MCM3AP, CTDP1, BABAM1, KLHL11, AP4E1, ARAF, CELF1, PAFAH1B1, MOCOS, FBN1, ABR, ARHGEF28, EGFR, TRIM16, POR, CRYBG1, FBX038, DMXL2, VPS16, IFT172, IFI16, RNF123, PELO, SL C39A14, PPP6C, TBK1, NGLY1, RASA3, IKBKB, SMS, GPD2, SHC1, MALT1, NDUFV1, FHOD1, SBNO2, SYDE1, RAPH1, FKBP15, ZC3HC1, TTC27, GTF3C2, UBASH3B, PML, ADAM 15, KLHDC10, MY D88, MED24, CC2D1A, DIP2B, APPL1, RINT1, MAEA, IFT57.

What is claimed is:

1. A method of treating cancer in a subject in need thereof, the method comprising administering to the subject a pharmaceutical composition comprising a therapeutically effective amount of at least one Leucine Zipper-like Transcription Regulator 1 (LZTR1) substrate inhibitor, wherein the cancer is associated with a germline or somatic loss-of-function mutation in a LZTR1 gene, wherein the mutation comprises one or more nucleotide insertion, deletion, or substitution mutations in the LZTR1 gene or any combination thereof.

2. The method of claim 1, wherein the mutation comprises a deletion of at least a portion of the LZTR1 gene, results in a V456G, P520L, L591R, R688C, R810W, or S813I mutation in LZTR1, or results in a H71R, P115L, S122L, R170Q, L187R, M202R, or M400R mutation in LZTR1.

3. The method of claim 1, wherein the mutation is located in a CUL-3-interacting domain or substrate-interacting domain of the LZTR1 gene.

4. The method of claim 1, wherein the cancer is glioblastoma multiforme (GBM), Schwannoma, hepatocellular cancer, esophagogastric cancer, colorectal cancer, breast carcinoma, prostate cancer, lung adenocarcinoma, clonal hematopoiesis disorder, or tumor predisposition syndrome.

5. The method of claim 1, wherein the cancer expresses higher levels of at least one LZTR1 substrate compared to a sample of non-cancerous tissue.

6. The method of claim 5, wherein the LZTR1 substrate is Epidermal Growth Factor Receptor (EGFR), AXL or RIT1.

7. The method of claim 1, wherein the LZTR1 substrate inhibitor comprises a small molecule inhibitor of a LZTR1 substrate, an antibody or antigen binding fragment thereof that specifically binds to a LZTR1 substrate, an antisense RNA or antisense DNA that decreases expression of a polypeptide of a LZTR1 substrate, a siRNA that specifically targets a LZTR1 substrate mRNA, a sgRNA that specifically targets a nucleotide sequence encoding a LZTR1 substrate, or a combination thereof.

8. The method of claim 1, wherein the LZTR1 substrate inhibitor comprises an EGFR inhibitor, an AXL inhibitor, or a RIT1 inhibitor.

9. The method of claim 8, wherein the LZTR1 substrate inhibitor comprises a small molecule inhibitor.

10. The method of claim 1, wherein the LZTR1 substrate inhibitor comprises afatinib or a pharmaceutically acceptable salt thereof, osimertinib or a pharmaceutically acceptable salt thereof, or bemcentinib or a pharmaceutically acceptable salt thereof.

11. The method of claim 1, wherein the LZTR1 substrate inhibitor comprises a small molecule EGFR inhibitor and the pharmaceutical composition comprises a second LZTR1 substrate inhibitor comprising a small molecule AXL inhibitor.

12. The method of claim 11, wherein the EGFR inhibitor comprises osimertinib or a pharmaceutically acceptable salt thereof and the AXL inhibitor comprises bemcentinib or a pharmaceutically acceptable salt thereof, or wherein the EGFR inhibitor comprises afatinib or a pharmaceutically acceptable salt thereof and the AXL inhibitor comprises bemcentinib or a pharmaceutically acceptable salt thereof.

13. The method of claim 1, wherein the pharmaceutical composition comprises a small molecule EGFR inhibitor and the method further comprises administering a second pharmaceutical composition comprising a small molecule AXL inhibitor.

14. The method of claim 13, wherein the pharmaceutical composition comprises osimertinib, or a pharmaceutically acceptable salt thereof and the second pharmaceutical composition comprises bemcentinib or a pharmaceutically acceptable salt thereof, or wherein the pharmaceutical composition comprises afatinib or a pharmaceutically acceptable salt thereof and the second pharmaceutical composition comprises bemcentinib or a pharmaceutically acceptable salt thereof.

15. The method of claim 13, wherein the pharmaceutical composition and the second pharmaceutical composition are administered to the subject at the same or different times.

16. The method of claim 1, wherein the subject is a human.

17. A pharmaceutical composition comprising a therapeutically effective amount of an EGFR inhibitor and an AXL inhibitor.

18. The pharmaceutical composition of claim 17, wherein the EGFR and AXL inhibitors are small molecules.

19. The pharmaceutical composition of claim 17, wherein the EGFR inhibitor comprises osimertinib or a pharmaceutically acceptable salt thereof and the AXL inhibitor comprises bemcentinib or a pharmaceutically acceptable salt thereof, or wherein the EGFR inhibitor comprises afatinib or a pharmaceutically acceptable salt thereof and the AXL inhibitor comprises bemcentinib or a pharmaceutically acceptable salt thereof.

20. The pharmaceutical composition of claim 17, wherein the pharmaceutical composition is for treating cancer in a subject wherein the cancer is associated with a loss-of-function mutation in a LZTR1 gene.

21. The pharmaceutical composition of claim 17, further comprising at least one pharmaceutically acceptable excipient, diluent, and/or carrier.

22. A method of decreasing growth of a solid tumor in a subject in need thereof, the method comprising administering to the subject a pharmaceutical composition comprising a therapeutically effective amount of at least one Leucine Zipper-like Transcription Regulator 1 (LZTR1) substrate inhibitor, wherein the composition decreases the size of the solid tumor, and wherein the tumor is associated with a somatic or germline loss-of-function mutation in a LZTR1 gene, wherein the mutation comprises one or more nucleotide insertion, deletion, or substitution mutations in the LZTR1 gene or any combination thereof.

23. The method of claim 1, further comprising determining that the cancer of the subject does not express a LZTR1 protein or comprises a nucleic acid sequence encoding a mutant LZTR1 protein before administering the pharmaceutical composition.
